(19) United States
(12) Patent Application Publication
(10) Pub. No.: US 2024/0228497 A1
(43) Pub. Date: Jul. 11, 2024

(54) Pyrrolo[2, 3-b]Pyrazine-Based Bifunctional Compounds as HPK1 Degraders and the Use Thereof

(71) Applicants: BeiGene, Ltd., Camana Bay (KY); BeiGene Switzerland GmbH, Basel (CH)

(72) Inventors: Sanjia Xu, Beijing (CN); Jing Li, Beijing (CN); Zhiwei Wang, Beijing (CN); Hanzi Sun, Beijing (CN); Ye Liu, Beijing (CN)

(73) Assignees: BeiGene, Ltd., Camana Bay (KY); BeiGene Switzerland GmbH, Basel (CH)

(21) Appl. No.: 18/422,518

(22) Filed: Jan. 25, 2024

Related U.S. Application Data
(63) Continuation of application No. PCT/CN2022/108923, filed on Jul. 29, 2022.

(30) Foreign Application Priority Data
Jul. 30, 2021 (WO) PCT/CN2021/109547

(51) Int. Cl. C07D 487/04; A61K 31/4985; A61K 31/513; A61P 35/00; C07D 471/04

(52) U.S. Cl. CPC C07D 487/04; A61K 31/4985; A61K 31/513; A61P 35/00; C07D 471/04

(57) ABSTRACT

Disclosed herein are novel pyrrolo[2,3-b]pyrazine-based bifunctional compounds formed by conjugating HPK1 inhibitor moieties with E3 ligase Ligand moieties, which function to recruit targeted proteins to E3 ubiquitin ligase for degradation, and methods of preparation and uses thereof.

### FIELD OF THE INVENTION

The disclosure herein provides pyrrolo[2, 3-b]pyrazine-based bifunctional compounds as well as their compositions and methods of use. The compounds disclosed herein have activity to degrade hematopoietic progenitor kinase 1 (HPK1) protein, therefore reduce or inhibit the function of HPK1. The compounds disclosed are useful in the treatment of various HPK1-mediated diseases including cancer.

### BACKGROUND OF THE INVENTION

HPK1 regulates diverse functions of various immune cells and its kinase activity has been shown to be induced upon activation of T cell receptors (TCR) [Liou, J., et al., Immunity, 2000. 12 (4): pp. 399-408], B cell receptors (BCR) [Liou, J., et al., Immunity, 2000. 12 (4): pp. 399-408], transforming growth factor receptor (TGF-βR) [Wang, W., et al., J Biol Chem, 1997. 272 (36): pp. 22771-5; Zhou, G., et al., J Biol Chem, 1999. 274 (19): pp. 13133-8], and Gs-coupled PGE2 receptors (EP2 and EP4) [Ikegami, R., et al., J Immunol, 2001. 166 (7): pp. 4689-96]. Overexpression of HPK1 suppresses TCR-induced activation of AP-1-dependent gene transcription in a kinase-dependent manner, suggesting that HPK1 is required to inhibit the Erk MAPK pathway [Liou, J., et al., Immunity, 2000. 12 (4): pp. 399-408] and this blockage is thought to be the inhibitory mechanism that negatively regulates TCR-induced IL-2 gene transcription [Sawasdikosol, S., et al., Immunol Res, 2012. 54: pp. 262-5].

In vitro HPK1-/- T cells have a lower TCR activation threshold, proliferate robustly, produce enhanced amounts of Th1 cytokines, the HPK1-/- mice experience more severe autoimmune symptoms [Sawasdikosol, S., et al., Immunol Res, 2012. 54: pp. 262-5]. In human, HPK1 was downregulated in peripheral blood mononuclear cells of psoriatic arthritis patients or T cells of systemic lupus erythematosus (SLE) patients [Batliwalla, F. M., et al., Mol Med, 2005. 11 (1-12): pp. 21-9], which indicated that attenuation of HPK1 activity may contribute to autoimmunity in patients. Furthermore, HPK1 may also control anti-tumor immunity via T cell-dependent mechanisms. In the PGE2-producing Lewis lung carcinoma tumor model, the tumors developed more slowly in HPK1 knockout mice as compared to wild-type mice [US patent application No. 2007/0087988]. HPK1 deficient T cells were more effective in controlling tumor growth and metastasis than wild-type T cells [Alzabin, S., et al., Cancer Immunol Immunother, 2010. 59 (3): pp. 419-29]. Similarly, BMDCs from HPK1 knockout mice were more efficient to mount a T cell response to eradicate Lewis lung carcinoma as compared to wild-type BMDCs [Alzabin, S., et al., J Immunol, 2009. 182 (10): pp. 6187-94].

It is believed that HPK1 negatively regulates T cell activation by the phosphorylation of the SH2 domain-containing leukocyte protein of 76 kDa (SLP76) at Ser376, which results in the recruitment of 14-3-3 proteins that leads to the proteolytic degradation of SLP76 [Shui, J-W., et al., Nat Immunol, 2007. 8 (1): pp. 84-91]. On top of the kinase activities, HPK1 protein may also have scaffolding functions. For example, HPK1 may compete with degranulation promoting adaptor protein (ADAP) to bind with SLP76, that inhibits T cell adhesion [Patzak, I. M., et al., Eur J Immunol, 2010. 40 (11): pp. 3220-5]. To degrade HPK1 protein may serve both purposes of hindering the kinase and scaffolding functions of HPK1 to enhance T cell activity. Also, the modality of protein degradation is expected to overcome potential resistance due to gene mutation and/or over-expression.

Proteolysis-targeting chimera (PROTAC) is a novel strategy for selective knockdown of target proteins by small molecules (Sakamoto K M et al., Proc Natl Acad Sci 2001, 98: pp. 8554-9 .; Sakamoto K. M. et al., Methods Enzymol. 2005;399: pp. 833-47.). PROTAC utilizes the ubiquitin-protease system to target a specific protein and induce its degradation in the cell (Zhou P. et al., Mol Cell. 2000;6(3): pp.751-6; Neklesa T. K. et al., Pharmacol Ther. 2017; 174:138-144; Lu M. et al., Eur J Med Chem. 2018; 146: pp. 251-9;).

WO2020227325 discloses several series of bivalent compounds as HPK1 degraders. However, there is a need to provide new HPK1 protein degraders to treat HPK1-mediated diseases, especially cancer.

### SUMMARY OF THE INVENTION

One objective of the present invention is to provide a series of proteolysis targeting chimera (PROTAC) compounds by conjugating HPK1 inhibitors with E3 ligase ligands, which function to recruit targeted proteins to E3 ubiquitin ligase for degradation, and to provide a method of the preparation and uses thereof. In particular, the present disclosure provides PROTAC compounds with Formula (I).

Aspect 1: A compound of Formula (I):

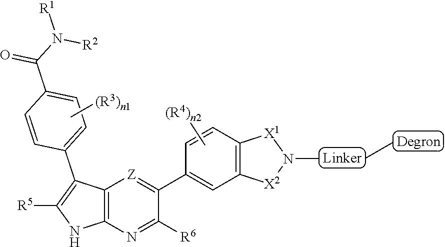

or a pharmaceutically acceptable salt thereof, or a stereoisomer thereof,
wherein
Z is selected from N or CR^{za};
   R^{za} is selected from hydrogen, —C₁₋₈alkyl, cycloalkyl;
R¹ and R² are each independently hydrogen, deuterium, —C₁₋₈alkyl, —C₂₋₈alkenyl, —C₂₋₈alkynyl, cycloalkyl, heterocyclyl, aryl, or heteroaryl, each of said —C₁₋₈alkyl, —C₂₋₈alkenyl, —C₂₋₈alkynyl, cycloalkyl, heterocyclyl, aryl, or heteroaryl is optionally substituted with at least one substituents R^{1a}; or
R¹ and R² together with the atom to which they are attached, form a 3- to 12-membered ring, said ring comprising 0, 1 or 2 additional heteroatoms independently selected from nitrogen, oxygen or optionally oxidized sulfur as ring member (s), said ring is optionally substituted with at least one substituents R^{1b};
   R^{1a} and R^{1b} are each independently —D (deuterium), halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{1c}, —SO₂R^{1c}, —SO₂NR^{1c}R^{1d}, —COR^{1c}, —CO₂R^{1c}, —CONR^{1c}R^{1d}, —C(═NR^{1c})NR^{1d}R^{1e}, —NR^{1c}R^{1d}, —NR^{1c}COR^{1d}, —NR^{1c}CONR^{1d}R^{1e}, —NR^{1c}CO₂R^{1c}, —NR^{1c}SONR^{1d}R^{1e}, —NR^{1c}SO₂NR^{1d}R^{1e}, or —NR^{1c}SO₂R^{1d}, each of said —C₁₋₈alkyl, —C₂₋₈alkenyl, —C₂₋₈alkynyl, cycloalkyl, heterocyclyl, aryl, or heteroaryl is optionally substituted with at least one substituents selected from halogen, —C₁₋₈alkyl, —OR^{1f}, —NR^{1f}R^{1g}, cycloalkyl, heterocyclyl, aryl, or heteroaryl;
      R^{1c}, R^{1d}, R^{1e}, R^{1f}, and R^{1g} are each independently hydrogen, —C₁₋₈alkyl, C₁₋₈alkoxy-C₁₋₈alkyl-, —C₂₋₈alkenyl, —C₂₋₈alkynyl, cycloalkyl, heterocyclyl, aryl, or heteroaryl;
n1 is 0, 1, 2, 3 or 4;
n2 is 0, 1, 2 or 3;
R³, at each of its occurrence, are independently halogen, —C₁₋₈alkyl, —C₂₋₈alkenyl, —C₂₋₈alkynyl, cycloalkyl, heterocyclyl, —C₁₋₈alkyl-heterocyclyl, —C₁₋₈alkyl-cycloalkyl, aryl, heteroaryl, oxo, —CN, —NO₂, —OR^{3a}, —SO₂R^{3a}, —SO₂NR^{3a}R^{3b}, —COR^{3a}, —CO₂R^{3a}, —CONR^{3a}R^{3b}, —C(═NR^{3a})NR^{3b}R^{3c}, —NR^{3a}R^{3b}, —NR^{3a}COR^{3b}, —NR^{3a}CONR^{3b}R^{3c}, —NR^{3a}CO₂R^{3b}, —NR^{3a}SONR^{3b}R^{3c}, —NR^{3a}SO₂NR^{3b}R^{3c}, or —NR^{3a}SO₂R^{3b}, each of said —C₁₋₈alkyl, —C₂₋₈alkenyl, —C₋₈alkynyl, cycloalkyl, heterocyclyl, —C₁₋₈alkyl-heterocyclyl, aryl, or heteroaryl is optionally substituted with at least one substituents R^{3d}; or
or when n1=2, two R³, when on adjacent carbon atoms of the phenyl ring, together with the two intervening carbon atoms to which they are attached, form a 5- to 8-membered ring comprising 0, 1 or 2 heteroatoms independently selected from nitrogen, oxygen or optionally oxidized sulfur as ring member (s);
   R^{3a}, R^{3b}, and R^{3c} are each independently hydrogen, —C₁₋₈alkyl, —C₂₋₈alkenyl, —C₂₋₈alkynyl, cycloalkyl, heterocyclyl, aryl, or heteroaryl, each of said —C₁₋₈alkyl, —C₂₋₈alkenyl, —C₂₋₈alkynyl, cycloalkyl, heterocyclyl, aryl, or heteroaryl is optionally substituted with at least one substituents R^{3e}; or
   (R^{3a} and R^{3b}), (R^{3b} and R^{3c}), or (R^{3c} and R^{3a}), together with the atom (s) to which they are attached, form a 3- to 12-membered ring, said ring comprising 0, 1 or 2 additional heteroatoms independently selected from nitrogen, oxygen or optionally oxidized sulfur as ring member (s), said ring is optionally substituted with at least one substituents R^{3e};
   R^{3d} and R^{3e} are each independently halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{3f}, —SO₂R^{3f}, —SO₂NR^{3f}R^{3g}, —COR^{3f}, —CO₂R^{3f}, —CONR^{3b}R^{3g}, —C(═NR^{3f})NR^{3g}R^{3h}, —NR^{3f}R^{3g}, —NR^{3f}COR^{3g}, —NR^{3f}CONR^{3g}R^{3h}, —NR^{3f}CO₂R^{3f}, —NR^{3f}SONR^{3f}R^{3g}, —NR^{3f}SO₂NR^{3g}R^{3h}, or —NR^{3f}SO₂R^{3g}, each of said —C₁₋₈alkyl, —C₂₋₈alkenyl, —C₂₋₈alkynyl, cycloalkyl, heterocyclyl, aryl, or heteroaryl is optionally substituted with at least one substituents selected from halogen, —C₁₋₈alkyl, —OR³ⁱ, —NR³ⁱR^{3j}, cycloalkyl, heterocyclyl, aryl, or heteroaryl;
      R^{3f}, R^{3g}, R^{3h}, R³ⁱ, and R^{3j} are each independently hydrogen, —C₁₋₈alkyl, —C₁₋₈alkoxy-—C₁₋₈alkyl-, —C₂₋₈alkenyl, —C₂₋₈alkynyl, cycloalkyl, heterocyclyl, aryl, or heteroaryl;
R⁴, R⁵ and R⁶ are each independently hydrogen, halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{4a}, —SO₂R^{4a}, —COR^{4a}, —CO₂R^{4a}, —CONR^{4a}R^{4b}, —C(═NR^{4a})NR^{4b}R^{4c}, —NR^{4a}R^{4b}, —NR^{4a}COR^{4b}, —NR^{4a}CONR^{4b}R^{4c}, —NR^{4a}CO₂R^{4b}, —NR^{4a}SONR^{4b}R^{4c}, —NR^{4a}SO₂NR^{4b}R^{4c}, or —NR^{4a}SO₂R^{4b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, —C₁₋₈alkyoxy, cycloalkyl, heterocyclyl, aryl, or heteroaryl;
   R^{4a}, R^{4b}, and R^{4c} are each independently hydrogen, —C₁₋₈alkyl, —C₂₋₈alkenyl, —C₂₋₈alkynyl, cycloalkyl, heterocyclyl, aryl, or heteroaryl;
X¹ and X² are each independently a single bond, —CR^{X1}R^{X2}, —CR^{X1}R^{X2}CR^{X3}R^{X4}, or —CR^{X1}R^{X2}CR^{X3}R^{X4} CR^{X5}R^{X6};
   wherein R^{X1}, R^{X2}, R^{X3}, R^{X4}, R^{X5}, R^{X6} and RX6 at each of its occurrence, are independently selected from hydrogen, —C₁₋₈alkyl, —C₂₋₈alkenyl, —C₂₋₈alkynyl, —C₃₋₈cycloalkyl, 3- to 8-membered heterocyclyl, —C₆₋₁₂aryl, 5- to 12-membered heteroaryl, each of said —C₁₋₈alkyl, —C₂₋₈alkenyl, —C₂₋₈alkynyl, —C₃₋₈cycloalkyl, 3- to 8-membered heterocyclyl, —C₆₋₁₂aryl or 5- to 12-membered heteroaryl is optionally substituted with at least one substituent RXa;
      each of said R^{Xa} is independently oxo, halogen, hydroxy, —C₁₋₈alkyl, —C₁₋₈alkoxy, —C₁₋₈alkyl, —C₂₋₈alkenyl, —C₂₋₈alkynyl, —C₁₋₈haloalkyl, —C₃₋₈cycloalkyl, 3- to 8-membered heterocyclyl, C₆₋₁₂aryl or 5- to 12-membered heteroaryl;
the Linker is a bond or a divalent linking group, and
the Degron is an E3 Ubiquitin ligase moiety.

Aspect 2. The compound according to Aspect 1, wherein the Degron moiety is selected from:

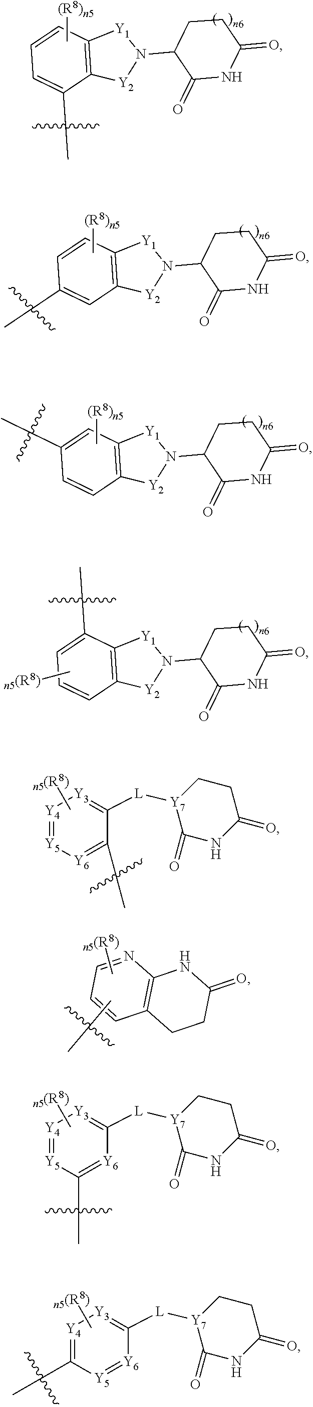

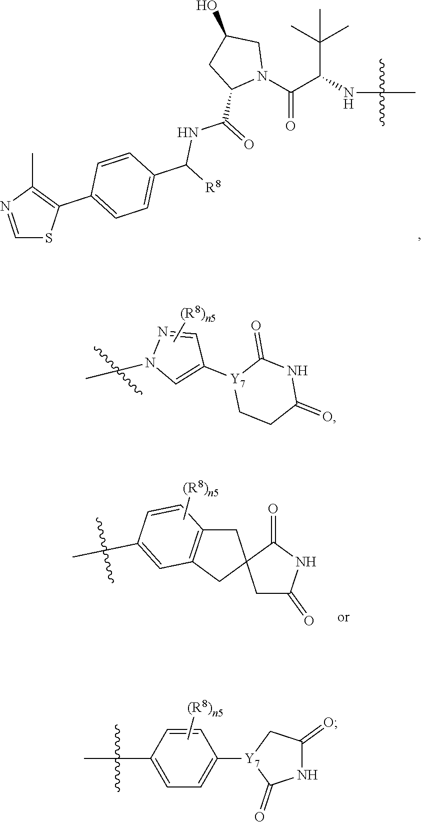

wherein
Y₁ and Y₂ are each independently —CH₂—, —NH— or —C(O)—;
Y_{3,} Y₄, Y₅ and Y₆ are each independently CH or N;
Y₇ is CH or N;
L is selected from a bond, —CH₂—, —O—, —NH— and —S—;
n5 is 0, 1, 2, 3, or 4;
n6 is 0, 1, or 2;
R⁸ is each independently hydrogen, halogen, —C₁₋₈alkyl, —C₂₋₈alkenyl, —C₂₋₈alkynyl, cycloalkyl, heterocyclyl, aryl, heteroaryl, —CN, —NO₂, —OR^{8a}, —SO₂R^{8a}, —COR^{8a}, —CO₂R^{8a}, —CONR^{8a}R^{8b}, —C(═NR^{8a})NR^{8b}R^{8c}, —NR^{8a}R^{8b}, —NR^{8a}COR^{8b}, —NR^{8a}CONR^{8b}R^{8c}, —NR^{8a}CO₂R^{8b}, —NR^{8a}SONR^{8b}R^{8c}, —NR^{8a}SO₂NR^{8b}R^{8c}, or —NR^{8al SO}₂R^{8b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, —C₁₋₈alkyoxy, cycloalkyl, heterocyclyl, aryl, or heteroaryl; and
R^{8a}, R^{8b}, and R^{8c} are each independently hydrogen, —C₁₋₈alkyl, —C₂₋₈alkenyl, —C₂₋₈alkynyl, cycloalkyl, heterocyclyl, aryl, or heteroaryl.

Aspect 3. The compound according to Aspect 2, wherein Degron moiety is selected from

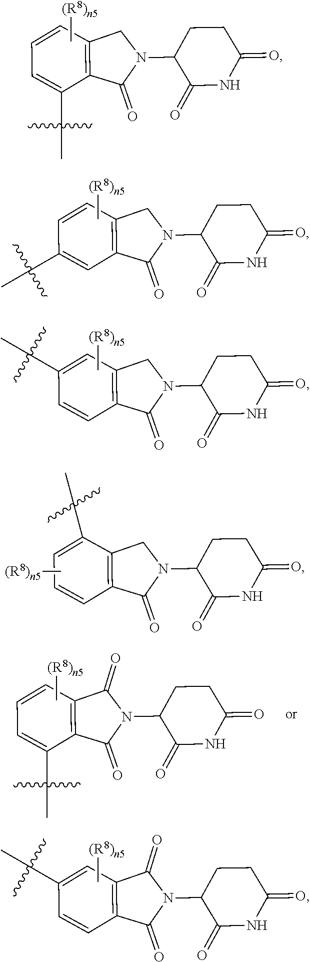

wherein R⁸ and n5 are defined as above.

Aspect 4. The compound according to Aspect 2, wherein Degron moiety is selected from

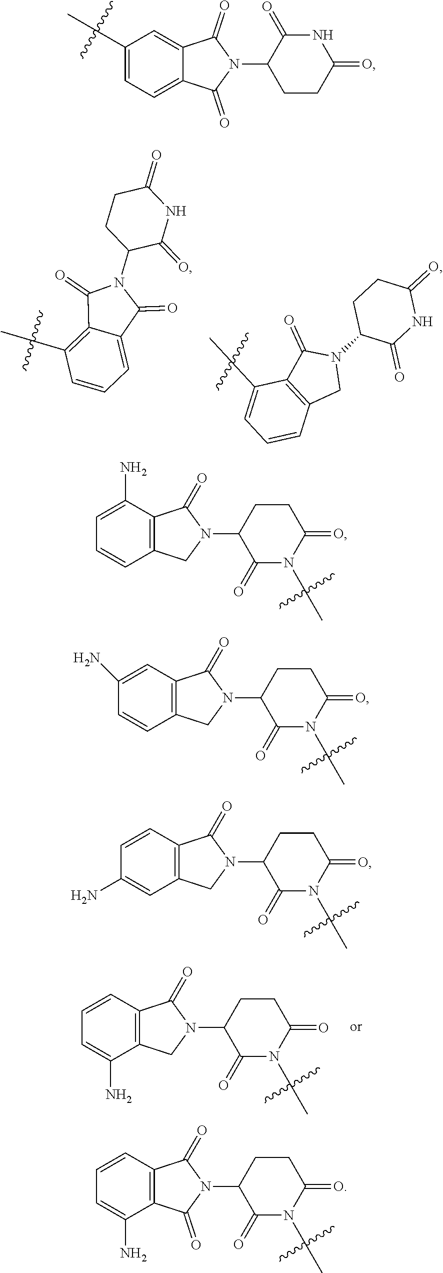

Aspect 5. The compound according to Aspect 2, wherein Degron moiety is selected from

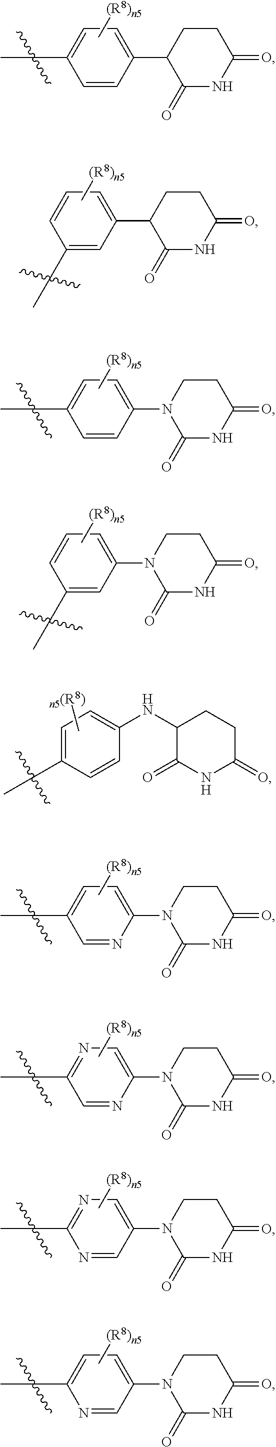

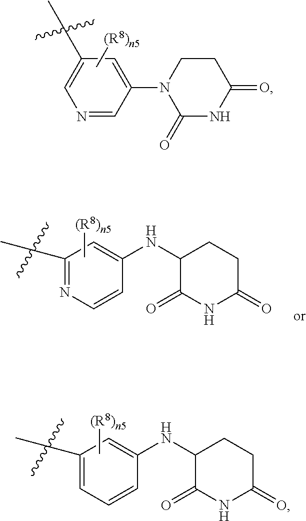

wherein R⁸ and n5 are defined as above.

Aspect 6. The compound according to Aspect 2, wherein Degron moicty is selected from

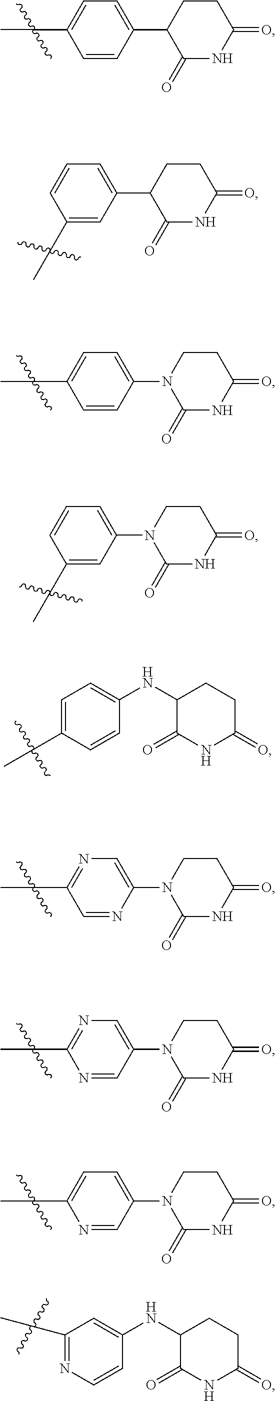

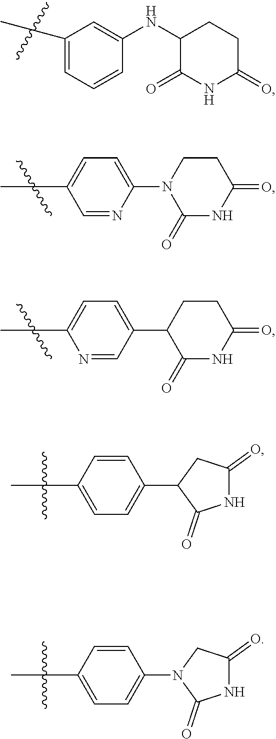 [SMILES: *c1cccc(NC2CCC(=O)NC2=O)c1 ; *c1ccc(N2CCC(=O)NC2=O)nc1 ; *c1ccc(C2CCC(=O)NC2=O)cn1 ; *c1ccc(C2CC(=O)NC2=O)cc1 ; *c1ccc(N2CC(=O)NC2=O)cc1]

Aspect 7. The compound according to Aspect 2, wherein R⁸ is hydrogen, —F, —Cl, —Br, —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —CN, —NO₂, —OR^{8a}, —SO₂R^{8a}, —COR^{8a}, —CO₂R^{8a}, —CONR^{8a}R^{8b}, —C(═NR^{8a})NR^{8b}R^{8c}, —NR^{8a}R^{8b}, —NR^{8a}COR^{8b}, —NR^{8a}CONR^{8b}R^{8c}, —NR^{8a}CO₂R^{8b}, NR^{8a}SONR^{8b}R^{8c}, —NR^{8a}SO₂NR^{8b}R^{8c}, or —NR^{8a}SO₂R^{8b}, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with —F, —Cl, —Br, —I, hydroxy, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl; and
R^{8a}, R^{8b}, and R^{8c} are each independently hydrogen, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl.

Aspect 8. The compound according to Aspect 2, wherein R⁸ is hydrogen, —F, —Cl, —Br, —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl or —CN.

Aspect 9. The compound according to Aspect 2, wherein R⁸ is hydrogen, —F, —Cl, —Br, —I, methyl, ethyl, propyl, butyl, pentyl, hexyl, cyclopropyl, cyclobutyl, cyclopentyl, cyclohexyl.

Aspect 10. The compound according to Aspect 1, wherein the

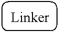

moiety is

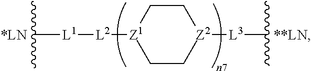

wherein *^{LN} refers to the position attached to the

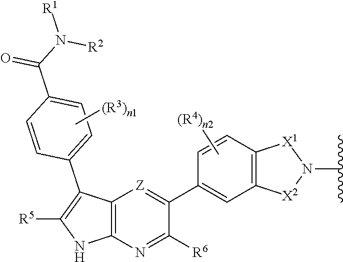

moiety, and **^{LN} refers to the position attached to the

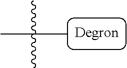

moiety;
Z¹ and Z² are each independently selected from CH or N;
n7 is 0 or 1;
L¹ is a single bond, —O—, —SO₂—, —C(O)—, —NR^{L1a}—, —C₃-C₈cycloalkylene-, *^{L1}—O—C₁₋₈alkylene-**^{L1}, *^{L1}—C₁₋₈alkylene-O—**^{L1}, *^{L1}—SO₂—C₁₋₈alkylene-**^{L1}, *^{L1}-C₁₋₈alkylene-SO₂-**^{L1}, *^{L1}—C(O)—C₁₋₈alkylene-**^{L1}, *^{L1}—C₁₋₈alkylene-C(O)—**^{L1}, *^{L1}—NR^{L1a}—C₁₋₈alkylene-**^{L1}, *^{L1}—C₁₋₈alkylene-NR^{L1a}—**^{L1}, *^{L1}—NR^{L1a}C(O)—**^{L1}, *^{L1}—C(O)NR^{L1a}—**^{L1}, *^{L1}—NR^{L1a}C(O)C₁₋₈alkylene-**^{L1a}, *^{L1}—C(O)NR^{L1a}C₁₋₈alkylene-**^{L1}, *^{L1}—C₁₋₈alkyleneNR^{L1a}C(O)—**^{L1}, *^{L1}—C₁₋₈alkyleneC(O)NR^{L1a}—**^{L1}, —C₁₋₈alkylene-, —C₂₋₈alkenylene-, —C₂₋₈alkynylene-, —[O(CR^{L1a}R^{L1b})ᵤ₁]ᵥ₁—, *^{L1}—CO—(CR^{L1a}R^{L1b})ᵤ₁—**^{L1}, *^{L1}—(CR^{L1a}R^{L1b})ᵤ₁—CO—**^{L1}*^{L1}—(CR^{L1a}R^{L1b})ᵤ₁—**^{L1}; each of said —C₃-C₈cycloalkylene-, *^{L1}—O—C₁₋₈alkylene-**^{L1}, *^{L1}—C₁₋₈alkylene-O—**^{L1}, *^{L1}—SO₂—C₁₋₈alkylene-**^{L1}, *^{L1}—C₁₋₈alkylene-SO₂—**^{L1}, *^{L1}—C(O)—C₁₋₈alkylene-**^{L1}, *^{L1}—C₁₋₈alkylene-C(O)—**^{L1}, *^{L1}—NR^{L1a}—C₁₋₈alkylene-**^{L1}, *^{L1}—C₁₋₈alkylene-NR^{L1a}—**^{L1}, *^{L1}—NR^{L1a}C(O)C₁₋₈alkylene-**^{L1}, *^{L1}—C(O)NR^{L1a}C₁₋₈alkylene-**^{L1}, *^{L1}—C₁₋₈alkyleneNR^{L1a}C(O)—**^{L1}, *^{L1}—C₁₋₈alkyleneC(O)NR^{L1a}—**^{L1}, —C₁₋₈alkylene-, —C₂₋₈alkenylene or —C₂₋₈alkynylene- are optionally substituted with at least one R^{L1c};
u1 and v1 are each independently selected from 1, 2, 3, 4, 5, 6, 7 or 8;
wherein *^{L1} refers to the position attached to the

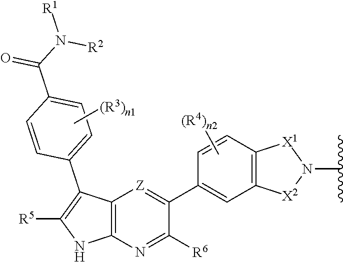

moiety, and
**^{L1} refers to the position attached to the

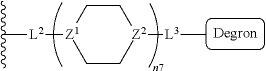

moiety;
L² is selected from a single bond, —O—, —SO₂—, —CO—, —NR^{L2a}—, —C₃-C₈cycloalkylene-, *^{L2}—O—C₁₋₈alkylene-**^{L2}, *^{L2}—C₁₋₈alkylene-O—**^{L2}, *^{L2}—SO₂—C₁₋₈alkylene-**^{L2}, *^{L2}—C₁₋₈alkylene-SO₂—**^{L2}, *^{L2}—CO—C₁₋₈alkylene-**^{L2}, *^{L2}—C₁₋₈alkylene-CO—**^{L2}, *^{L2}—NR^{L2a}—C₁₋₈alkylene-**^{L2}, *^{L2}—C₁₋₈alkylene-NR^{L2a}—**^{L2}, *^{L2}, *^{L2}—NR^{L2a}—C(O)—**^{L2}, *^{L2}—C(O)NR^{L2a}—**^{L2}, *^{L2}—NR^{L2a}—C(O)C₁₋₈alkylene-**^{L2}, *^{L2}—C(O)NR^{L2a}—C₁₋₈alkylene-**^{L2}, *^{L2}—C₁₋₈alkyleneNR^{L2a}C(O)-**^{L2}, *^{L2}—C₁₋₈alkyleneC(O)NR^{L2a}—**^{L2}, —C₁₋₈alkylene-, —C₂₋₈alkenylene-, —C₂₋₈alkynylene-, —[O(CR^{L2a}—R^{L2b})ᵤ₂]ᵥ₂—, *^{L2}—CO—(CR^{L2a}R^{L2b})ᵤ₂—**^{L2}, *^{L2}—(CR^{L2a}R^{L2b})ᵤ₂—CO—**^{L2}, *^{L2}—(CR^{L2a}R^{L2b})ᵤ₂—**^{L2}; each of said —C₃-C₈cycloalkylene-, *^{L2}—O—C₁₋₈alkylene-**^{L2}, *^{L2}-C₁₋₈alkylene-O—**^{L2}, *^{L2}—SO₂—C₁₋₈alkylene-**^{L2}, *^{L2}—C₁₋₈alkylene-SO₂—**^{L2}, *^{L2}—CO—C₁₋₈alkylene-**^{L2}, *^{L2}—C₁₋₈alkylene-CO —**^{L2}, *^{L2}—NR^{L2a}—C₁₋₈alkylene-**^{L2}, *^{L2}—C₁₋₈alkylene-NR^{L2a}—, **^{L2}, *^{L2}—NR^{L2a}—C(O)C₁₋₈alkylene-**^{L2}, *^{L2}—C(O)NR^{L2a}C₁₋₈alkylene-**^{L2}, *^{L2}—C₁₋₈alkyleneNR^{L2a}C(O)—**^{L2}, *^{L2}—C₁₋₈alkyleneC(O)NR^{L2a}—**^{L2}, —C₁₋₈alkylene-, —C₂₋₈alkenylene or —C₁₋₈alkynylene- are optionally substituted with at least one substituent R^{L2c};
u2 and v2 are each independently selected from 1, 2, 3, 4, 5, 6, 7 or 8;
wherein *^{L2} refers to the position attached to the

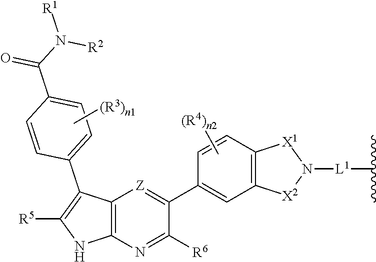

moiety, and **^{L2} refers to the position attached to the

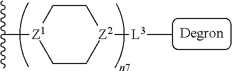

moiety;
L₃ is selected from a single bond, —O—, —SO₂—, —CO—, —NR^{L3a}—, —C₃-C₈cycloalkylene-, *^{L3}—O—C₁₋₈alkylene-**^{L3}, *^{L3}—C₁₋₈alkylene-O—**^{L3}, *^{L3}—SO₂—C₁₋₈alkylene-**^{L3}, *^{L3}—C₁₋₈alkylene-SO₂—**^{L3}, *^{L3}—CO—C₁₋₈alkylene-**^{L3}, *^{L3}—C₁₋₈alkylene-CO—**^{L3}, *^{L3}—NR^{L3a}—C₁₋₈alkylene-**^{L3}, *^{L3}—C₁₋₈alkylene-NR^{L3a}—**^{L3}, *^{L3}—NR^{L3a}C(O)—**^{L3}, *^{L3}—C(O)NR^{L3a}—**^{L3}, *^{L3}-NR^{L3a}C(O)C₁₋₈alkylene-**^{L3}, *^{L3}C(O)NR^{L3a}C₁₋₈alkylene-**^{L3}, *^{L3}—C₁₋₈alkyleneNR^{L3a}C(O)—**^{L3}, *^{L3}—C₁₋₈alkyleneC(O)NR^{L3a}—**^{L3}, —C₁₋₈alkylene-, —C₂₋₈alkenylene-, —C₂₋₈alkynylene-, —[O(CR^{L3a}R^{L3b})ᵤ₃]ᵥ₃—, —*^{L3}—CO—(CR^{L3a}R^{L3b})ᵤ₃—**^{L3}, *^{L3}—(CR^{L3a}R^{L3b})ᵤ₃—CO—**^{L3}, *^{L3}—(CR^{L3a}R^{L3b})ᵤ₃—**^{L3}; each of said —C₃-C₈cycloalkylene-, *^{L3}—O—C₁₋₈alkylene-**^{L3}, *^{L3}—C₁₋₈alkylene-O—**^{L3}, *^{L3}—SO₂—C₁₋₈alkylene-**^{L3}, *^{L3}—C₁₋₈alkylene-SO₂—**^{L3}, *^{L3}—CO—C₁₋₈alkylene-**^{L3}, *^{L3}—C₁₋₈alkylene-CO—**^{L3}, *^{L3}—NR^{L3a}—C₁₋₈alkylene-**^{L3}, *^{L3}—C₁₋₈alkylene-NR^{L3a}—**^{L3}, *^{L3}—NR^{L3a}C(O)C₁₋₈alkylene-**^{L3}, *^{L3}—C(O)NR^{L3a}C₁₋₈alkylene-**^{L3}, *^{L3}—C₁₋₈alkyleneNR^{L3a}C(O)—**^{L3}, *^{L3}—C₁₋₈alkyleneC(O)NR^{L3a}—**^{L3}, —C₁₋₈alkylene-, C₂₋₈alkenylene-, C₂₋₈alkynylene-, are optionally substituted with at least one substituent R^{L3c};
u3 and v3 are each independently selected from 1, 2, 3, 4, 5, 6, 7 or 8;
wherein *^{L3} refers to the position attached to the

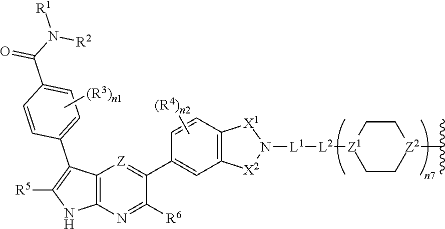

moiety, and **^{L3} refers to the position attached to the

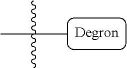

moiety;
R^{L1a}, R^{L1b}, R^{L1}c, R^{L2a}, R^{L2b}, R^{L2c}, R^{L3a}, R^{L3b} and R^{L3c} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or membered heteroaryl is optionally substituted with at least one substituent R^{L3d};
   R^{L3d} is halogen, hydroxy, —C₁₋₈alkyl, -haloC₁₋₈alkyl, C₁₋₈alkoxy, —C₂₋₈alkenyl, —C₁₋₈alkynyl, cycloalkyl, heterocyclyl, aryl or heteroaryl.

Aspect 11. The compound according to Aspect 10, wherein L¹ is a single bond, —O—, —SO₂—, —C(O)—, —NH—, —N(CH₃)—, —N(C₂H₅)—, —N(C₃H₇)—, *^{L1}—O—CH₂—**^{L1}, *^{L1}—O—C₂H₄—**^{L1}, *^{L1}—O—C₃H₆—**^{L1}, *^{L1}—O—C₄H₈—**^{L1}, *^{L1}—CH₂—O—**^{L1}, *^{L1}—C₂H₄—O—**^{L1}, *^{L1}—C₃H₆—O—**^{L1}, *^{L1}—C₄H₈—O—**^{L1}, *^{L1}—SO₂—CH₂—**^{L1}, *^{L1}—SO₂—C₂H₄—**^{L1}, *^{L1}—SO₂—C₃H₆—**^{L1}, *^{L1}—SO₂—C₄H₈—**^{L1}, *^{L1}—CH₂—SO₂—**^{L1}, *^{L1}—C₂H₄—SO₂—**^{L1}, *^{L1}—C₃H₆—SO₂—**^{L1}, *^{L1}—C₄H₈—SO₂—**^{L1}, *^{L1}—C(O)—CH₂—**^{L1}, *^{L1}—C(O)—C₂H₄—**^{L1}, *^{L1}—C(O)—C₃H₆—**^{L1}, *^{L1}—C(O)—C₄H₈—**^{L1}, *^{L1}—CH₂—C(O)—**^{L1}, *^{L1}—C₂H₄—C(O)—**^{L1}, *^{L1}—C₃H₆—C(O)—**^{L1}, *^{L1}—C₄H₈—C(O)—**^{L1}, *^{L1}—NH—CH₂—**^{L1}, *^{L1}—NH—C₂H₄—**^{L1}, *^{L1}—NH—C₃H₆—**^{L1}, *^{L1}—NH—C₄H₈**^{L1}, *^{L1}—CH₂—NH—**^{L1}, *^{L1}—C₂H₄—NH—**^{L1}, *^{L1}—C₃H₆—NH —**^{L1}, *^{L1}—C₄H₈—NH—**^{L1}, *^{L1}—NHC(O)—**^{L1}, *^{L1}—C(O)NH—**^{L1}, *^{L1}—N(CH₃)C(O)—**^{L1}, *^{L1}—C(O)N(CH₃)—**^{L1}, *^{L1}—N(C₂H₅)C(O)—**^{L1}, *^{L1}—C(O)N(C₂H₅)—**^{L1}, *^{L1}—N(C₃H₇)C(O)—**^{L1}, *^{L1}—C(O)N(C₃H₇)—**^{L1}, *^{L1}—NHC(O)CH₂—**^{L1}, *^{L1}—NHC(O)C₂H₄—**^{L1}, *^{L1}—NHC(O)C₃H₆—**^{L1}, *^{L1}—NHC(O)C₄H₈—**^{L1}, *^{L1}—C(O)NHCH₂—**^{L1}, *^{L1}—C(O)NHC₂H₄—**^{L1}, *^{L1}—C(O)NHC₃H₆—**^{L1}, *^{L1}—C(O)NHC₄H₈—**^{L1}, *^{L1}—CH₂NHC(O)—**^{L1}, *^{L1}—C₂H₄NHC(O)—**^{L1}, *^{L1}—C₃H₆NHC(O)—**^{L1}, *^{L1}—C₄H₈NHC(O)—**^{L1}, *^{L1}—CH₂C(O)NH—**^{L1}, *^{L1}—C₂H₄C(O)NH—**^{L1}, *^{L1}—C₃H₆C(O)NH—**^{L1}, *^{L1}—C₄H₈C(O)NH—**^{L1}, —CH₂—, —C₂H₄—, —C₃H₆—, —C₄H₈—, —O(CH₂)₂—, —[O(CH₂)₂]₂—, —[O(CH₂)₂]₃—, —[O(CH₂)₂]₄— or —[O(CH₂)₂]₅—.

Aspect 12. The compound according to Aspect 10, wherein L¹ is *^{L1}—CH₂—C(O)—**^{L1}, *^{L1}—CH₂CH₂—C(O)—**^{L1}, *^{L1}—CH₂CH₂CH₂—C(O)—**^{L1}, *^{L1}—CH₂—NH—**^{L1}, *^{L1}—CH₂CH₂—NH—**^{L1}, *^{L1}—CH₂CH₂CH₂—NH—**^{L1}, *^{L1}—CH₂NHC(O)—**^{L1}, *^{L1}—CH₂CH₂NHC(O)—**^{L1}, *^{L1}—CH₂CH₂CH₂NHC(O)—**^{L1}, *^{L1}—CH₂C(O)NH—**^{L1}, *^{L1}—CH₂CH₂C(O)NH—**^{L1}, *^{L1}—CH₂CH₂CH₂C(O)NH—**^{L1}, —CH₂—, —CH₂CH₂—, —CH₂CH₂CH₂—.

Aspect 13. The compound according to Aspect 10, wherein L² is a single bond, —O—, —SO₂—, —C(O)—, —NH—, —N(CH₃)—, —N(C₂H₅)—, —N(C₃H₇)—, *^{L2}—O—CH₂—**^{L2}, *^{L2}—O—C₂H₄—**^{L2}, *^{L2}—O—C₃H₆—** ^{L2}, *^{L2}—O—C₄H₈—**^{L2}, *^{L2}—CH₂—O—**^{L2}, *^{L2}—C₂H₄—O—**^{L2}, *^{L2}—C₃H₆—O—**^{L2}, *^{L2}—C₄H₈—O—**^{L2}, *^{L2}—SO₂—CH₂—**^{L2}, *^{L2}—SO₂—C₂H₄—**^{L2}, *^{L2}—SO₂—C₃H₆—**^{L2}, *^{L2}—SO₂—C₄H8 - **^{L2}, *^{L2}—CH₂—SO₂—**^{L2}, *^{L2}—C₂H₄—SO₂—**^{L2}, *^{L2}—C₃H₆—SO₂—**^{L2}, *^{L2}—C₄H₈—SO₂—**^{L2}, *^{L2}—C(O)—CH₂—**^{L2}, *^{L2}—C(O)—C₂H₄—**^{L2}, *^{L2}—C(O)—C₃H₆—**^{L2}, *^{L2}—C(O)—C₄H₈—**^{L2}, *^{L2}—CH₂—C(O)—**^{L2}, *^{L2}—C₂H₄—C(O)—**^{L2}, *^{L2}—C₃H₆—C(O)—**^{L2}, *^{L2}—C₄H₈—C(O)—**^{L2}, *^{L2}—NH—CH₂—**^{L2}, *^{L2}—NH—C₂H₄—**^{L2}, *^{L2}—NH—C₃H₆—**^{L2}, *^{L2}—NH—C₄H₈—**^{L2}, *^{L2}—CH₂—NH—**^{L2}, *^{L2}—C₂H₄—NH—**^{L2}, *^{L2}—C₃H₆—NH—**^{L2}, *^{L2}—C₄H₈—NH—**^{L2}, *^{L2}—NHC(O)—**^{L2}, *^{L2}—C(O)NH—**^{L2}, *^{L2}—N(CH₃)C(O)—**^{L2}, *^{L2}—C(O)N(CH₃)—**^{L2}, *^{L2}—N(C₂H₅)C(O)—**^{L2}, *^{L2}—C(O)N(C₂H₅)—**^{L2}, *^{L2}—N(C₃H₇)C(O)—**^{L2}, *^{L2}—C(O)N(C₃H₇)—**^{L2}, *^{L2}—NHC(O)CH₂—**^{L2}, *^{L2}—NHC(O)C₂H₄—**^{L2}, *^{L2}—NHC(O)C₃H₆—**^{L2}, *^{L2}—NHC(O)C₄H₈—**^{L2}, *^{L2}—C(O)NHCH₂—**^{L2}, *^{L2}—C(O)NHC₂H₄—**^{L2}, *^{L2}-C(O)NHC₃H₆—**^{L2}, *^{L2}—C(O)NHC₄H₈—**^{L2}, *^{L2}—CH₂NHC(O)—**^{L2}, *^{L2}—C₂H₄NHC(O)—**^{L2}, *^{L2}—C₃H₆NHC(O)—**^{L2}, *^{L2}—C₄H₈NHC(O)—**^{L2}, *^{L2}—CH₂C(O)NH—**^{L2}, *^{L2}—C₂H₄C(O)NH—**^{L2}, *^{L2}—C₃H₆C(O)NH—**^{L2}, *^{L2}—C₄H₈C(O)NH—**^{L2}, —CH₂—, —C₂H₄—, —C₃H₆—, —C₄H₈—, —O(CH₂)₂—, —[O(CH₂)₂]₂—, —[O(CH₂)₂]₃—, —[O(CH₂)₂]₄— or —[O(CH₂)₂]₅—.

Aspect 14. The compound according to Aspect 10, wherein L² is a single bond, —C(O)—, —NH—, *^{L2}—NHC(O)—**^{L2}, *^{L2}—C(O)NH—**^{L2}, *^{L2}—NH—CH₂—**^{L2}, *^{L2}—NH—CH₂CH₂—**^{L2}, *^{L2}—NH—CH₂CH₂CH₂—**^{L2}, *^{L2}—C(O)—CH₂ —**^{L2}, *^{L2}—C(O)—CH₂CH₂—**^{L2}, *^{L2}—C(O)—CH₂CH₂CH₂—**^{L2}, *^{L2}—NHC(O)CH₂—**^{L2}, *^{L2}—NHC(O)CH₂CH₂—**^{L2}, *^{L2}—NHC(O)CH₂CH₂CH₂—**^{L2}, *^{L2}—C(O)NHCH₂—**^{L2}, *^{L2}—C(O)NHCH₂CH₂—**^{L2}, or *^{L2}—C(O)NHCH₂CH₂CH₂—**^{L2}.

Aspect 15. The compound according to Aspect 10, wherein L³ is a single bond, —O—,—SO₂—, —C(O)—, —NH—, —N(CH₃)—, —N(C₂H₅)—, —N(C₃H₇)—, *^{L3}—O—CH₂—**^{L3}, *^{L3}—O—C₂H₄—**^{L3}, *^{L3}—O—C₃H₆—**^{L3}, *^{L3}—O—C₄H₈—**^{L3}, *^{L3}—CH₂—O—**^{L3}, *^{L3}—C₂H₄—O—**^{L3}, *^{L3}—C₃H₆—O—**^{L3}, *^{L3}—C₄H₈—O—**^{L3}, *^{L3}—SO₂—CH₂—**^{L3}, *^{L3}—SO₂—C₂H₄—**^{L3}, *^{L3}—SO₂—C₃H₆—**^{L3}, *^{L3}—SO₂—C₄H₈—**^{L3}, *^{L}3—CH₂—SO₂—**^{L3}, *^{L3}—C₂H₄—SO₂—**^{L3}, *^{L3}—C₃H₆—SO₂—**^{L3}, *^{L3}—C₄H₈—SO₂—**^{L3}, *^{L3}—C(O)—CH₂—**^{L3}, *^{L3}—C(O)—C₂H₄—**^{L3}, *^{L3}—C(O)—C₃H₆—**^{L3}, *^{L3}—C(O)—C₄H₈—**^{L3}, *^{L3}—CH₂—C(O)—**^{L3}, *^{L3}—C₂H₄—C(O)—**^{L3}, *^{L3}—C₃H₆—C(O)—**^{L3}, *^{L3}—C₄H₈—C(O)—**^{L3}, *^{L3}—NH—CH₂—**^{L3}, *^{L3}—NH—C₂H₄—**^{L3}, *^{L3}—NH—C₃H₆—**^{L3}, *^{L3}—NH—C₄H₈—**^{L3}, *^{L3}—CH₂—NH—**^{L3}, *^{L3}—C₂H₄—NH—**^{L3}, *^{L3}—C₃H₆—NH—**^{L3}, *^{L3}—C₄H₈—NH—**^{L3}, *^{L3}—NHC(O)—**^{L3}, *^{L3}—C(O)NH—**^{L3}, *^{L3}—N(CH₃)C(O)—**^{L3}, *^{L3}—C(O)N(CH₃)—**^{L3}, *^{L3}—N(C₂H₅)C(O)—**^{L3}, *^{L3}—C(O)N(C₂H₅)—**^{L3}, *^{L3}—N(C₃H₇)C(O)—**^{L3}, *^{L3}—C(O)N(C₃H7)—**^{L3}, *^{L3}—NHC(O)CH₂—**^{L3}, *^{L3}—NHC(O)C₂H₄—**^{L3}, *^{L3}—NHC(O)C₃H₆—**^{L3}, *^{L3}—NHC(O)C₄H₈—**^{L3}, *^{L3}—C(O)NHCH₂—**^{L3}, *^{L3}—C(O)NHC₂H₄—**^{L3}, *^{L3}—C(O)NHC₃H₆—**^{L3}, *^{L3}—C(O)NHC₄H₈—**^{L3}, *^{L3}—CH₂NHC(O)—**^{L3}, *^{L3}—C₂H₄NHC(O)—**^{L3}, *^{L3}—C₃H₆NHC(O)—**^{L3}, *^{L3}—C₄H₈NHC(O)—**^{L3}, *^{L3}—CH₂C(O)NH—**^{L3}, *^{L3}—C₂H₄C(O)NH—**^{L3}, *^{L3}—C₃H₆C(O)NH—**^{L3}, *^{L3}—C₄H₈C(O)NH—**^{L3}, —CH₂—, —C₂H₄—, —C₃H₆—, —C₄H₈—, —O(CH₂)₂—, —[O(CH₂)₂]₂—, —[O(CH₂)₂]₃—, —[O(CH₂)₂]₄— or —[O(CH₂)₂]₅—.

Aspect 16. The compound according to Aspect 10, wherein L³ is a single bond.

Aspect 17. The compound according to Aspect 10, wherein

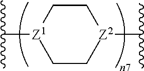

moiety is

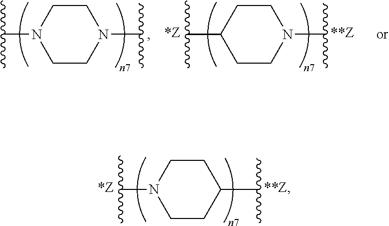

wherein *^{LN} refers to the position attached to the

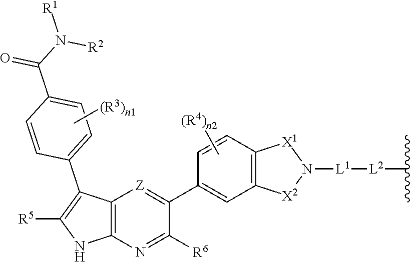

moiety, and **^{LN} refers to the position attached to the

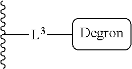

moiety.

Aspect 18. The compound according to Aspect 1, wherein the

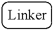

moiety is

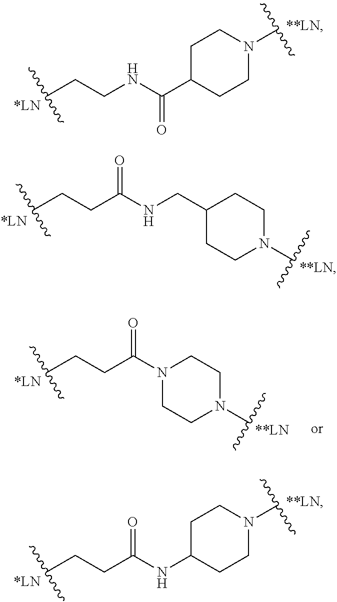 [SMILES: *CCNC(=O)C1CCN(*)CC1 ; *CCC(=O)NCC1CCN(*)CC1 ; *CCC(=O)N1CCN(*)CC1 ; *CCC(=O)NC1CCN(*)CC1]

wherein *^{LN} refers to the position attached to the

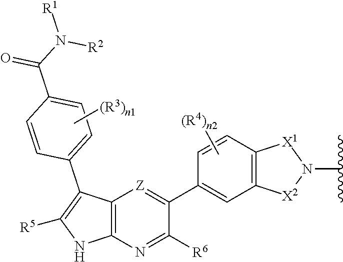

moiety, and **^{LN} refers to the position attached to the

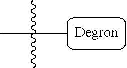

moiety.

Aspect 19. The compound according to Aspect 1, wherein R¹ and R² are each independently hydrogen, deuterium, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one substituents R^{1a}; or
R¹ and R² together with the atom (s) to which they are attached, form a 3-, 4-, 5-, 6-, 7- or 8-membered ring, said ring comprising 0, 1 or 2 additional heteroatoms independently selected from nitrogen, oxygen or optionally oxidized sulfur as ring member (s), said ring is optionally substituted with at least one substituents R^{1b};
R₁ₐ and R^{1b} are each independently —D, —F, —Cl, —Br, —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, oxo, —CN, —NO₂, —OR^{1c}, —SO₂R^{1c}, —SO₂NR^{1c}R^{1d}, —COR^{1c}, —CO₂R^{1c}, —CONR^{1c}R^{1d}, —C(═NR^{1c})NR^{1d}R^{1e}, —NR^{1c}R^{1d}, —NR^{1c}COR^{1d}, —NR^{1c}CONR^{1d}R^{1e}, —NR^{1c}CO₂R^{1c}, —NR^{1c}SONR^{1c}R^{1d}, —NR^{1c}SO₂NR^{1d}R^{1e}, or —NR^{1c}SO₂R^{1d}, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one substituents selected from —F, —Cl, —Br, —I, methyl, ethyl, propyl, butyl, pentyl, hexyl, heptyl, octyl, -ORIf, —NR1R18, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl;
   R^{1c}, R^{1d}R^{1e}, R^{1f}, and R^{1g} are each independently hydrogen, methyl, ethyl, propyl, butyl, pentyl, hexyl, heptyl, octyl, Ci galkoxy—C salkyl-, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl.

Aspect 20. The compound according to Aspect 1, wherein R¹ and R² are each independently hydrogen, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl; each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one OH; or
R¹ and R² together with the atom (s) to which they are attached, form a 3-, 4-, 5-, 6-, 7- or 8-membered ring, said ring comprising 0, 1 or 2 additional heteroatoms independently selected from nitrogen, oxygen or optionally oxidized sulfur as ring member (s).

Aspect 21. The compound according to Aspect 1, wherein R¹ and R² are each independently hydrogen, —CD₃, methyl, ethyl, n-propyl, iso-propyl, n-butyl, sec-butyl, iso-butyl,tert-butyl, 2-hydroxypropyl, hydroxymethyl, 1-hydroxyethyl, 2-hydroxyethyl, 1-hydroxypropyl, 3-hydroxypropyl, 1-hydroxybutyl, 2-hydroxybutyl, 3-hydroxybutyl, or 4-hydroxybutyl.

Aspect 22. The compound according to Aspect 1, wherein R³, at each of its occurrence, are independently —F, —Cl, —Br, —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —CH₂-heterocyclyl, —CH₂CH₂-heterocyclyl, —CH₂CH₂CH₂-heterocyclyl, —CH₂CH₂CH₂CH₂-heterocyclyl, —CH₂-cycloalkyl, —CH₂CH₂-cycloalkyl, —CH₂CH₂CH₂-cycloalkyl, —CH₂CH₂CH,CH₂-cycloalkyl, oxo, —CN, —NO₂, —OR^{3a}, —SO₂R^{3a}, —SO₂NR^{3a}R^{3b}, —COR₃ₐ, —CO₂R^{3a}, —CONR^{3a}R^{3b}, —C(═NR^{3a})NR^{3b}R^{3c}, —NR^{3a}R^{3b}, —NR^{3a}COR^{3b}, —NR^{3a}CONR^{3b}R^{3c}, —NR^{3a}CO₂R^{3b}, —NR^{3a}SONR^{3b}R^{3c}, —NR^{3a}SO₂NR^{3b}R^{3c}, or —NR^{3a}SO₂R^{3b}, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —CH₂-heterocyclyl, —CH₂CH₂-heterocyclyl, —CH₂CH₂CH₂-heterocyclyl, —CH₂CH₂CH₂CH₂-heterocyclyl, —CH₂-cycloalkyl, —CH,CH₂-cycloalkyl, —CH₂CH₂CH₂-cycloalkyl or —CH₂CH CH₂CH₂-cycloalkyl is optionally substituted with at least one substituents R^{3d}; or
or two R³, when on adjacent carbon atoms of the phenyl ring, together with the two intervening carbon atoms to which they are attached, form a 5- to 8-membered ring comprising 0, 1 or 2 heteroatoms independently selected from nitrogen, oxygen or optionally oxidized sulfur as ring member (s);
R^{3a}, R^{3b}, and R^{3c} are cach independently hydrogen, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl. pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one substituents R^{3e}; or
(R^{3a} and R^{3b}), (R^{3b} and R^{3c}), or (R^{3c} and R^{3a}), together with the atom (s) to which they are attached, form a 3- to 12-membered ring, said ring comprising 0, 1 or 2 additional heteroatoms independently selected from nitrogen, oxygen or optionally oxidized sulfur as ring member (s), said ring is optionally substituted with at least one substituents R^{3e};
   R^{3d} and R^{3e} are cach independently —F, —Cl, —Br, —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl. pyrimidinyl, pyrazinyl, oxo, —CN, —NO₂, —OR^{3f}, —SO₂R^{3f}, —SO₂NR^{3f}R^{3g}, —COR^{3f}, —CO₂R^{3f}, —CONR^{3f}R^{3g}, —C(═NR^{3f})NR^{3g}R^{3h}, —NR^{3f}R^{3g}, —NR^{3f}COR^{3g}, —NR^{3f}CONR^{3g}R^{3h}, -—NR^{3f}CO₂R^{3f}, —NR^{3f}SONR^{3f}R^{3g}, —NR^{3f}SO₂NR^{3g}R^{3h}, or —NR^{3f}SO₂R^{3g}, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one substituents selected from —F, —Cl, —Br, —I, methyl, ethyl, propyl, butyl, pentyl, hexyl, heptyl, octyl, —OR³ⁱ, —NR³ⁱR^{3j}, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl;
      R^{3f}, R^{3g}, R^{3h}, R³ⁱ, and R^{3j} are each independently hydrogen, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, C₁₋₈alkoxy-C₁₋₈alkyl-

Aspect 23. The compound according to Aspect 1, wherein R³, at each of its occurrence, are independently —F, —Cl, —Br, —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —CH₂-heterocyclyl, —CH₂CH₂-heterocyclyl, —CH₂CH₂CH₂-heterocyclyl, —CH₂CH₂CH₂CH₂-heterocyclyl, —CH₂-cycloalkyl, —CH₂CH₂-cycloalkyl, —CH₂CH₂CH₂-cycloalkyl, —CH CH₂CH₂CH₂-cycloalkyl, oxo, —CN, —NO₂, —OR^{3a}, —SO₂R^{3a}, —SO₂NR^{3a}R^{3b}, —COR^{3a}, —CO₂R^{3a}, —CONR^{3a}R^{3b}, —C(═NR^{3a})NR^{3b}R^{3c}, —NR^{3a}R^{3b}, —NR^{3a}COR^{3b}, —NR^{3a}CONR^{3b}R^{3c}, —NR^{3a}CO₂R^{3b}, —NR^{3a}SONR^{3b}R^{3c}, —NR^{3a}SO₂NR^{3b}R^{3c}, or —NR^{3a}SO₂R^{3b}, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —CH₂-heterocyclyl, —CH₂CH₂-heterocyclyl, —CH₂CH₂CH₂-heterocyclyl, —CH₂CH₂CH₂CH₂-heterocyclyl, —CH₂-cycloalkyl, —CH₂CH₂-cycloalkyl, —CH₂CH₂CH₂-cycloalkyl or —CH₂CH₂CH₂CH₂-cycloalkyl is optionally substituted with at least one substituents R^{3d}; or
two R³, when on adjacent carbon atoms of the phenyl ring, together with the two intervening carbon atoms to which they are attached, form a 5- to 8-membered ring comprising 0, 1 or 2 heteroatoms independently selected from nitrogen, oxygen or optionally oxidized sulfur as ring member (s);
R^{3a}, R^{3b}, and R^{3c} are cach independently hydrogen, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one substituents R^{3e}; or
(R^{3a} and R^{3b}), (R^{3b}and R^{3c}), or (R^{3c}and R^{3a}), together with the atom (s) to which they are attached, form a 3- to 12-membered ring, said ring comprising 0, 1 or 2 additional heteroatoms independently selected from nitrogen, oxygen or optionally oxidized sulfur as ring member (s), said ring is optionally substituted with at least one substituents R^{3e};
   R^{3d} and R^{3e} are each independently —F, —Cl, —Br, —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, oxo, —CN, —NO₂, —OR^{3f}, —SO₂R^{3f}, —SO₂NR^{3f}R^{3g}, —COR^{3f}, —CO₂R^{3f}f, —CONR_{3f}R^{3g}, —C(═NR^{3f})NR^{3g}R^{3h}, —NR^{3f}R^{3g}, —NR^{3f}COR^{3g}, —NR^{3f}CONR^{3g}R^{3h}, —NR^{3f}CO₂R^{3f}, —NR^{3f}SONR^{3f}R^{3g}, —NR^{3f}SO₂NR^{3g}R^{3h}, or —NR^{3f}SO₂R^{3g}, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one substituents selected from —F, —Cl, —Br, —I, methyl, ethyl, propyl, butyl, pentyl, hexyl, heptyl, octyl, —OR³ⁱ, —NR³ⁱR^{3j}, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl;
      R^{3f}, R^{3g}, R^{3h}, R³ⁱ, and R^{3j} are each independently hydrogen, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, Ci galkoxy—C galkyl.

Aspect 23. The compound according to Aspect 1, wherein R³, at each of its occurrence, is selected from methyl, ethyl, propyl, butyl, pentyl, hexyl, heptyl, octyl, —F, —Cl, —Br, —I, —CN, OR^{3a} or —NR^{3a}CONR^{3b}R^{3c}; said —C₁₋₈alkyl is optionally substituted with at least one substituents R^{3d}; R^{3a}, R^{3b} and R^{3c} are each independently hydrogen, or —C₁₋₈alkyl (preferably methyl); R^{3d} is each independently —F, —Cl, —Br, —I, methyl, ethyl, propyl, butyl, pentyl, hexyl, heptyl or octyl.

Aspect 24. The compound according to Aspect 1, wherein R³ is —F, —Cl, —Br, —I, OH, CN, —CH₃, —C₂H₅, —CH₂CH₂CH₃, —CH(CH₃)CH₃, —CH₂CH₂CH₂CH₃, —CH(CH₃)CH₂CH₃, —CH₂CH(CH₃)CH₃, —C(CH₃)₃, —CH₂F, —CHF₂, —CF₃ or —NHCOCH₃.

Aspect 25. The compound according to Aspect 1, wherein

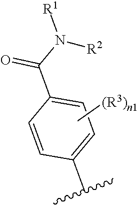

moiety is

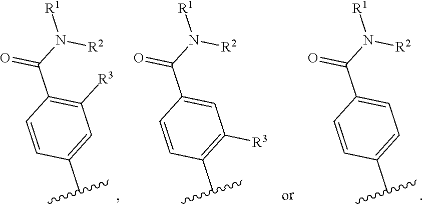

Aspect 26. The compound according to Aspect 1, wherein

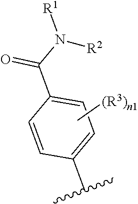

moiety is

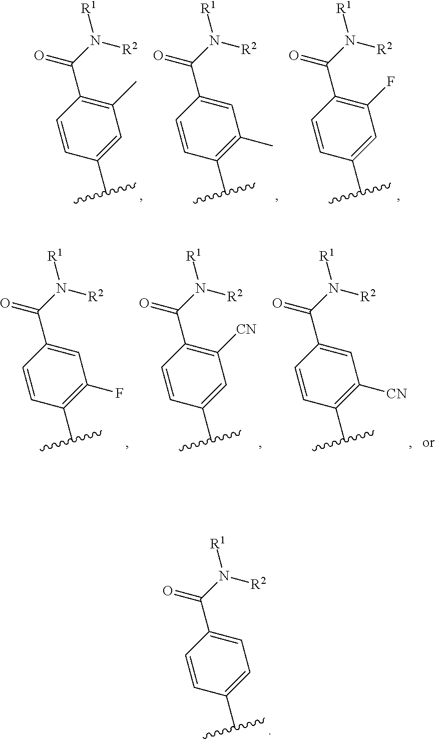

Aspect 27. The compound according to Aspect 1, wherein R⁴, R⁵ and R⁶ are each independently hydrogen, —F, —Cl, —Br, —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, oxo, —CN, —NO₂, —OR^{4a}, —SO₂R^{4a}, —COR^{4a}, —CO₂R^{4a}, —CONR^{4a}R^{4b}, —C(═NR^{4a})NR^{4b}R^{4b}, —NR^{4a}R^{4b}, —NR^{4a}COR^{4b}, —NR^{4a}CONR^{4b}R^{4c}, —NR^{4a}CO₂R^{4b}, —NR^{4a}SONR^{4b}R^{4c}, —NR^{4a}SO₂NR^{4b}R^{4c}, or —NR^{4a}SO₂R^{4b}, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with —F, —Cl, —F, —I, hydroxy, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl;
R^{4a}, R^{4b}, and R^{4c} are cach independently hydrogen, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyraziny.

Aspect 28. The compound according to Aspect 1, wherein R⁵ and R⁶ are each independently hydrogen or —C₁₋₈alkyl (preferably hydrogen or methyl).

Aspect 29. The compound according to Aspect 1, wherein R+is from methyl, ethyl, propyl, butyl, pentyl, hexyl, heptyl, octyl, —OR^{4a} or —NR^{4a}R^{4b}, said methyl, ethyl, propyl, butyl, pentyl, hexyl, heptyl or octyl is optionally substituted with at least one substituents -OH, —F, —Cl, —Br, —I, methyl, ethyl, propyl, butyl, pentyl, hexyl, heptyl or octyl;
R^{4a} and R^{4b} are cach hydrogen, methyl, ethyl, propyl, butyl, pentyl, hexyl, heptyl or octyl; preferably
R⁴ is selected from —CH₃, —OCH₃, —NHCH₃, —CHF₂, or —C(CH₃)₂OH.

Aspect 30. The compound according to Aspect 1, wherein X¹ and X² are each independently a single bond, —CH₂—, —CH₂CH₂—, —CH₂CH₂CH₂—.

Aspect 31. The compound according to Aspect 30, wherein when X1 is —CH₂—, X² is —CH₂CH₂—; or when X¹ is —CH₂CH₂—, X² is —CH₂—; or when X² is a single bond, X² is —CH₂CH₂CH₂—; or when X¹ is —CH₂CH₂CH₂—, X² is a single bond.

Aspect 32. The compound according to Aspect 1, wherein R^{X1}, R^{X2}, R^{X3}, R^{X4}, R^{X5} and R^{X6} each is selected from hydrogen, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, cach of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl is optionally substituted with at least one substituent R^{Xa}; each of said R^{Xa} are independently oxo, —F, —Cl, —Br, —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl.

Aspect 33. The compound according to Aspect 32, wherein R^{Xa} is selected from oxo, —F, —Cl, —Br, —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl.

Aspect 34. The compound according to Aspect 33, wherein R^{Xa} is selected from oxo, —F, —Cl, —Br, —I, methyl, ethyl, n-propyl, iso-propyl, n-butyl, sec-butyl, iso-butyl, tert-butyl, cyclopropyl, cyclobutyl, cyclopentyl.

Aspect 35. The compound according to Aspect 1. wherein the compound is selected from

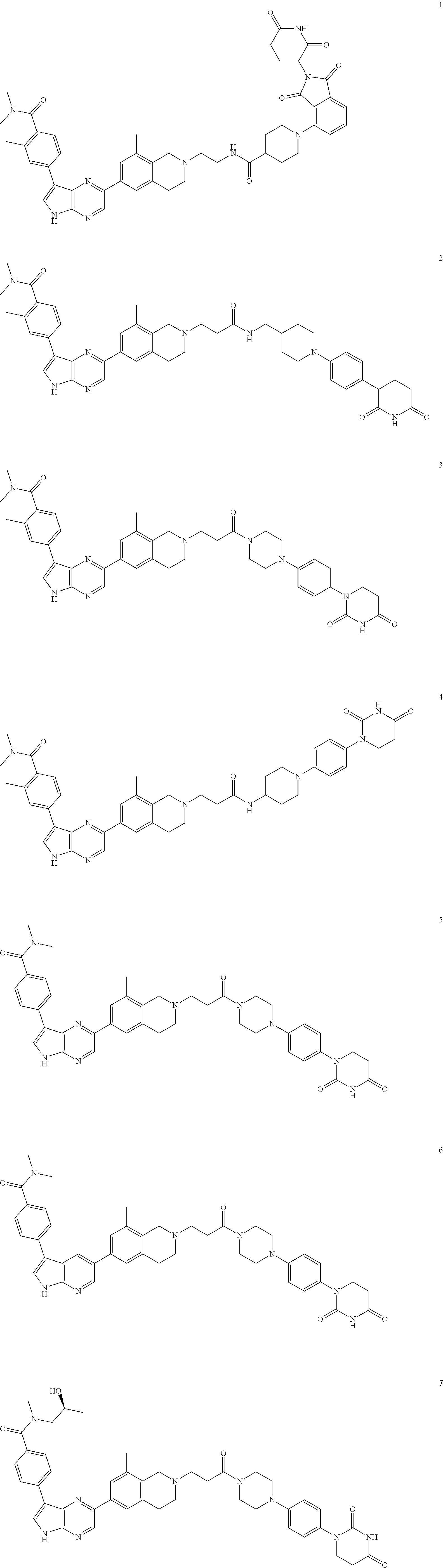

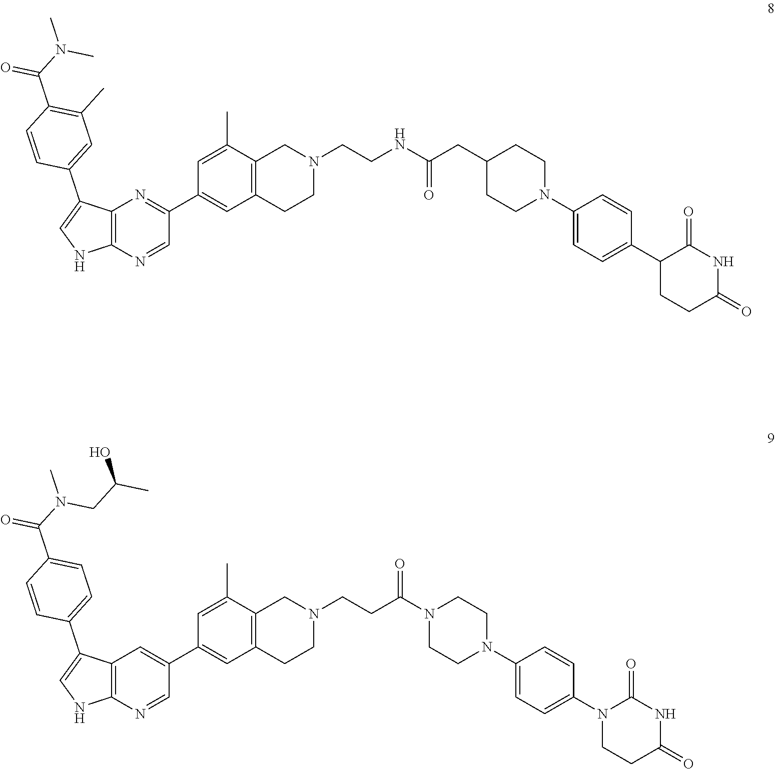 [SMILES: Cc1cc(-c2c[nH]c3ncc(-c4cc(C)c5c(c4)CCN(CCNC(=O)CC4CCN(c6ccc(C7CCC(=O)NC7=O)cc6)CC4)C5)nc23)ccc1C(=O)N(C)C ; Cc1cc(-c2cnc3[nH]cc(-c4ccc(C(=O)N(C)C[C@H](C)O)cc4)c3c2)cc2c1CN(CCC(=O)N1CCN(c3ccc(N4CCC(=O)NC4=O)cc3)CC1)CC2]

In the second aspect, disclosed herein is a pharmaceutical composition comprising the compound disclosed herein, or a pharmaceutically acceptable salt thereof, and at least one pharmaceutically acceptable carrier or excipient.

In the third aspect, disclosed herein is a method of decreasing HPK1 activity by inhibition and/or protein degradation, which comprises administering to an individual the compound disclosed herein, or a pharmaceutically acceptable salt thereof, including the compound of formula (I) or the specific compounds exemplified herein.

In the fourth aspect, disclosed herein is a method of treating a disease or disorder in a patient comprising administering to the patient a therapeutically effective amount of the compound disclosed herein, or a pharmaceutically acceptable salt thereof as an HPK1 degrader, wherein the compound disclosed herein includes the compound of formula (I) or the specific compounds exemplified herein. In some embodiments, the disease or disorder is associated with inhibition of HPK1. Preferably, the disease or disorder is cancer.

### DETAILED DESCRIPTION OF THE INVENTION

### Definitions

The following terms have the indicated meaning throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “alkyl” refers to a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include without limitation to methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1, 1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2, 3-dimethyl-2-butyl and 3, 3-dimethyl-2-butyl groups.

The term “propyl” refers to 1-propyl or n-propyl (“n-Pr”), 2-propyl or isopropyl (“i-Pr”).

The term “butyl” refers to 1-butyl or n-butyl (“n-Bu”), 2-methyl-1-propyl or isobutyl (“i-Bu”), 1-methylpropyl or s-butyl (“s-Bu”), 1, 1-dimethylethyl or t-butyl (“t-Bu”).

The term “pentyl” refers to 1-pentyl, 2-pentyl, 3-pentyl, 2-methyl-2-butyl, 3-methyl-2-butyl, 3-methyl-1-butyl, 2-methyl-1-butyl.

The term “hexyl” refers to 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2, 3-dimethyl-2-butyl and 3, 3-dimethyl-2-butyl.

The term “halogen” refers to fluoro (F), chloro (CI), bromo (Br) and iodo (I).

The term “haloalkyl” refers to an alkyl group in which one or more hydrogens are replaced by one or more halogen atoms such as fluoro, chloro, bromo, and iodo. Examples of the haloalkyl include without limitation to haloC₁₋₈alkyl, haloC₁₋₆alkyl or halo C₁₋₄alkyl, such as —CF₃, —CH₂Cl, —CH₂CF₃, —CHCl₂, —CF₃, and the like.

The term “alkenyl” refers to a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include without limitation to ethenyl or vinyl, prop-1-enyl, prop-2-enyl, 2-methylprop-1-enyl, but-1-enyl, but-2-enyl, but-3-enyl, buta-1, 3-dienyl, 2-methylbuta-1, 3-dienyl, hex-1-enyl, hex-2-enyl, hex-3-enyl, hex-4-enyl, and hexa-1, 3-dienyl groups.

The term “alkynyl” refers to a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include without limitation to ethynyl, 1-propynyl. 2-propynyl (propargyl), 1-butynyl, 2-butynyl, and 3-butynyl groups.

The term “cycloalkyl” refers to a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, examples of the saturated monocyclic cycloalkyl group, e.g., C₃₋₈cycloalkyl, include without limitation to cyclopropyl, cyclobutyl, cyclopentyl, cyclohexyl, cycloheptyl, and cyclooctyl groups. In a preferred embedment, the cycloalkyl is a monocyclic ring comprising 3 to 6 carbon atoms (abbreviated as C₃₋₆ cycloalkyl), including but not limited to cyclopropyl, cyclobutyl, cyclopentyl, and cyclohexyl.

Examples of the bicyclic cycloalkyl groups include those having from 7 to 12 ring atoms arranged as a fused bicyclic ring selected from [4, 4], [4, 5], [5, 5], [5, 6] and [6, 6] ring systems, or as a bridged bicyclic ring selected from bicyclo[2.2.1 ]heptane, bicyclo[2.2.2]octane, and bicyclo[3.2.2]nonane. Further examples of the bicyclic cycloalkyl groups include those arranged as a bicyclic ring selected from [5, 6] and [6, 6] ring systems.

The term “spiro cycloalkyl” refers to a cyclic structure which contains carbon atoms and is formed by at least two rings sharing one atom. The term “7 to 12 membered spiro cycloalkyl” refers to a cyclic structure which contains 7 to 12 carbon atoms and is formed by at least two rings sharing one atom.

The term “fused cycloalkyl” refers to a bicyclic cycloalkyl group as defined herein which is saturated and is formed by two or more rings sharing two adjacent atoms.

The term “bridged cycloalkyl” refers to a cyclic structure which contains carbon atoms and is formed by two rings sharing two atoms which are not adjacent to each other. The term “7 to 10 membered bridged cycloalkyl” refers to a cyclic structure which contains 7 to 12 carbon atoms and is formed by two rings sharing two atoms which are not adjacent to each other.

The term “cycloalkenyl” refers to non-aromatic cyclic alkyl groups of from 3 to 10 carbon atoms having single or multiple rings and having at least one double bond and preferably from 1 to 2 double bonds. In one embodiment, the cycloalkenyl is cyclopentenyl or cyclohexenyl, 1-cyclopent-1-enyl, 1-cyclopent-2-enyl, 1-cyclopent-3-enyl, 1-cyclohex-1-enyl, 1-cyclohex-2-enyl, 1-cyclohex-3-enyl, cyclohexadienyl. preferably cyclohexenyl.

The term “fused cycloalkenyl” refers to a bicyclic cycloalkyl group as defined herein which contain at least one double bond and is formed by two or more rings sharing two adjacent atoms.

The term “cycloalkynyl” refers to non-aromatic cycloalkyl groups of from 5 to 10 carbon atoms having single or multiple rings and having at least one triple bond.

The term “fused cycloalkynyl” refers to a bicyclic cycloalkyl group as defined herein which contains at least one triple bond and is formed by two or more rings sharing two adjacent atoms.

The term a “benzo fused cycloalkyl” is a bicyclic fused cycloalkyl in which a 4- to 8-membered monocyclic cycloalkyl ring fused to a benzene ring. For example, a benzo fused cycloalkyl is

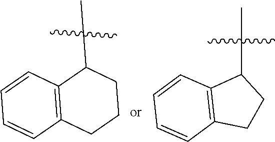

wherein the wavy lines indicate the points of attachment.

The term a “benzo fused cycloalkenyl” is a bicyclic fused cycloalkenyl in which a 4- to 8-membered monocyclic cycloalkenyl ring fused to a benzene ring.

The term a “benzo fused cycloalkynyl ” is a bicyclic fused cycloalkynyl in which a 4- to 8-

membered monocyclic cycloalkynyl ring fused to a benzene ring.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄₋₆ cycloalkenyl, 2, 3-dihydro-1H-indenyl, 1H-indenyl, 1, 2, 3, 4-tetralyl, 1, 4-dihydronaphthyl, etc. Preferred embodiments are 8 to 9 membered fused rings, which refer to cyclic structures containing 8 to 9 ring atoms within the above examples.

The term “aryl” used alone or in combination with other terms refers to a group selected from:
a) 5- and 6-membered carbocyclic aromatic rings, e.g., phenyl;
b) bicyclic ring systems such as 7 to 12 membered bicyclic ring systems, wherein at least one ring is carbocyclic and aromatic, e.g., naphthyl and indanyl; and,
c) tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeable throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include without limitation to phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” refers to a bicyclic aryl ring as defined herein. The typical bicyclic fused aryl is naphthalene.

The term “heteroaryl” refers to a group selected from:
a) 5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
b) 7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
c) 11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

Specifically, the term “bicyclic fused heteroaryl” refers to a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through cither ring.

Representative examples of bicyclic fused heteroaryl include without limitation to the following groups: benzisoxazolyl, benzodiazolyl, benzofuranyl, benzofurazanyl, benzofuryl, benzoimidazolyl, benzoisothiazolyl, benzothiadiazolyl, benzothiazolyl, benzothienyl, benzothiophenyl, benzotriazolyl, benzoxadiazolyl, benzoxazolyl, furopyridinyl, furopyrrolyl, imidazopyridinyl, imidazopyridyl, imidazothiazolyl, indazolyl, indolizinyl, indolyl, isobenzofuryl, isoindolyl, isoquinolinyl (or isoquinolyl), naphthyridinyl, phthalazinyl, pteridinyl, purinyl, pyrazinopyridazinyl, pyrazolopyridinyl, pyrazolopyrimidinyl, pyrazolopyridyl, pyrazolotriazinyl, pyridazolopyridyl, pyrrolopyridinyl, quinazolinyl, quinolinyl (or quinolyl), quinoxalinyl, thiazolopyridyl, thienopyrazinyl, thienopyrazolyl, thienopyridyl, thienopyrrolyl, thienothienyl, or triazolopyridyl.

The term a “benzo fused heteroaryl” is a bicyclic fused heteroaryl in which a 5- to 7-membered (preferably, 5- or 6-membered) monocyclic heteroaryl ring as defined herein fused to a benzene ring.

The terms “aromatic heterocyclic ring” and “heteroaryl” are used interchangeably throughout the disclosure herein. In some embodiments, a monocyclic or bicyclic aromatic heterocyclic ring has 5-, 6-, 7-, 8-, 9- or 10-ring forming members with 1, 2, 3, or 4 heteroatom ring members independently selected from nitrogen (N), sulfur (S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur (S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur (S) and oxygen (O). In some embodiments, the monocyclic or bicyclic aromatic heterocyclic ring is an 8- to 10-membered heteroaryl ring, which is bicyclic and which has 1 or 2 heteroatom ring members independently selected from nitrogen, sulfur and oxygen.

Examples of the heteroaryl group or the monocyclic or bicyclic aromatic heterocyclic ring include. but are not limited to, (as numbered from the linkage position assigned priority 1) pyridyl (such as 2-pyridyl, 3-pyridyl, or 4-pyridyl), cinnolinyl, pyrazinyl, 2, 4-pyrimidinyl, 3, 5-pyrimidinyl, 2, 4-imidazolyl, imidazopyridinyl, isoxazolyl, oxazolyl, thiazolyl, isothiazolyl, thiadiazolyl (such as 1, 2, 3-thiadiazolyl, 1, 2, 4-thiadiazolyl, or 1, 3, 4-thiadiazolyl), tetrazolyl, thienyl (such as thien-2-yl, thien-3-yl), triazinyl, benzothienyl, furyl or furanyl, benzofuryl, benzoimidazolyl, indolyl, isoindolyl, oxadiazolyl (such as 1, 2, 3-oxadiazolyl, 1, 2, 4-oxadiazolyl, or 1, 3, 4-oxadiazolyl), phthalazinyl, pyrazinyl, pyridazinyl, pyrrolyl, triazolyl (such as 1, 2, 3-triazolyl, 1, 2, 4-triazolyl, or 1, 3, 4-triazolyl), quinolinyl, isoquinolinyl, pyrazolyl, pyrrolopyridinyl (such as 1H-pyrrolo[2, 3-b]pyridin-5-yl), pyrazolopyridinyl (such as 1H-pyrazolo[ 3, 4-b]pyridin-5-yl), benzoxazolyl (such as benzo[d]oxazol-6-yl), pteridinyl, purinyl, 1-oxa-2, 3-diazolyl, 1-oxa-2, 4-diazolyl, 1-oxa-2, 5-diazolyl, 1-oxa-3, 4-diazolyl, 1-thia-2, 3-diazolyl, 1-thia-2, 4-diazolyl, 1-thia-2, 5-diazolyl, 1-thia-3, 4-diazolyl, furazanyl (such as furazan-2-yl, furazan-3-yl), benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), and indazolyl (such as 1H-indazol-5-yl).

“Heterocyclyl”, “heterocycle” or “heterocyclic” are interchangeable and refer to a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “optionally oxidized sulfur” used herein refer to S, SO or SO₂.

The term “monocyclic heterocyclyl” refers to monocyclic groups in which at least one ring member (e.g., 1-3 heteroatoms, 1 or 2 heteroatom(s)) is a heteroatom selected from nitrogen, oxygen or optionally oxidized sulfur. A heterocycle may be saturated or partially saturated.

Exemplary monocyclic 4 to 9-membered heterocyclyl groups include without limitation to pyrrolidin-1-yl, pyrrolidin-2-yl, pyrrolidin-3-yl, imidazolidin-2-yl, imidazolidin-4-yl, pyrazolidin-2-yl, pyrazolidin-3-yl, piperidin-1-yl, piperidin-2-yl, piperidin-3-yl, piperidin-4-yl, 2, 5-piperazinyl, pyranyl, morpholinyl, morpholino, morpholin-2-yl, morpholin-3-yl, oxiranyl, aziridin-1-yl, aziridin-2-yl, azocan-1-yl, azocan-2-yl, azocan-3-yl, azocan-4-yl, azocan-5-yl, thiiranyl, azetidin-1-yl, azetidin-2-yl, azctidin-3-yl, oxctanyl, thictanyl, 1, 2-dithictanyl, 1, 3-dithictanyl, dihydropyridinyl, tetrahydropyridinyl, thiomorpholinyl, thioxanyl, piperazinyl, homopiperazinyl, homopiperidinyl, azepan-1-yl, azepan-2-yl, azepan-3-yl, azepan-4-yl, oxepanyl, thicpanyl, 1, 4-oxathianyl, 1, 4-dioxepanyl, 1, 4-oxathicpanyl, 1, 4-oxaazepanyl, 1, 4-dithicpanyl, 1, 4-thiazepanyl and 1, 4-diazepanyl, 1, 4-dithianyl, 1, 4-azathianyl, oxazepinyl, diazepinyl, thiazepinyl, dihydrothicnyl, dihydropyranyl, dihydrofuranyl, tetrahydrofuranyl, tetrahydrothicnyl, tetrahydropyranyl, tetrahydrothiopyranyl. 1-pyrrolinyl, 2-pyrrolinyl, 3-pyrrolinyl, indolinyl, 2H-pyranyl, 4H-pyranyl, 1, 4-dioxanyl, 1, 3-dioxolanyl, pyrazolinyl, pyrazolidinyl, dithianyl, dithiolanyl, pyrazolidinyl, imidazolinyl, pyrimidinonyl, or 1, 1-dioxo-thiomorpholinyl.

The term “spiro heterocyclyl” refers to a 5 to 20-membered polycyclic heterocyclyl with rings connected through one common carbon atom (called a spiro atom), comprising one or more heteroatoms selected from nitrogen, oxygen or optionally oxidized sulfur as ring members, with the remaining ring members being carbon. One or more rings of a spiro heterocyclyl group may contain one or more double bonds, but none of the rings has a completely conjugated pi-electron system. Preferably a spiro heterocyclyl is 6 to 14-membered, and more preferably 7 to 12-membered. According to the number of common spiro atoms. a spiro heterocyclyl could be mono-spiro heterocyclyl, di-spiro heterocyclyl, or poly-spiro heterocyclyl, and preferably refers to mono-spiro heterocyclyl or di-spiro heterocyclyl, and more preferably 4-membered/3-membered. 4-membered/4-membered, 3-membered/5-membered, 4-membered/5-membered. 4-membered/6-membered. 5-membered/5-membered, or 5-membered/6-membered mono-spiro heterocyclyl. Representative examples of spiro heterocyclyls include without limitation to the following groups: 2, 3-dihydrospiro[indene-1, 2′-pyrrolidine] (e.g., 2, 3-dihydrospiro[ indene-1, 2′-pyrrolidine]-1′-yl), 1, 3-dihydrospiro[indene-2, 2′-pyrrolidine] (e.g., 1, 3-dihydrospiro[indene-2, 2′-pyrrolidine]-1′-yl), azaspiro[2.4]heptane (e.g., 5-azaspiro[2.4]heptane-5-yl), 2-oxa-6-azaspiro[3.3 ]heptane (e.g., 2-oxa-6-azaspiro[3.3]heptan-6-yl), azaspiro[3.4 ]octane (e.g., 6-azaspiro[3.4 ]octane-6- yl), 2-oxa-6-azaspiro[3.4 ]octane (e.g., 2-oxa-6-azaspiro[3.4 ]octane-6-yl), azaspiro[3.4 ]octane (e.g., 6-azaspiro[3.4 ]octan-6-yl), azaspiro[3.4]octane (e.g., 6-azaspiro[3.4 ]octan-6-yl), 1, 7-dioxaspiro[4.5]decane, 2-oxa-7-aza-spiro[4.4]nonane (e.g., 2-oxa-7-aza-spiro[4.4 ]non-7-yl), 7-oxa-spiro[3.5 ]nonyl and 5-oxa-spiro[2.4]heptyl,

The term “fused heterocyclyl” refers to a 5 to 20-membered polycyclic heterocyclyl group, wherein each ring in the system shares an adjacent pair of atoms (carbon and carbon atoms or carbon and nitrogen atoms) with another ring. comprising one or more heteroatoms selected from nitrogen, oxygen or optionally oxidized sulfur as ring members, with the remaining ring members being carbon. One or more rings of a fused heterocyclic group may contain one or more double bonds, but the fused heterocyclic group does not have a completely conjugated pi-electron system. Preferably, a fused heterocyclyl is 6 to 14-membered, and more preferably 7 to 12-membered, or 7- to 10-membered. According to the number of membered rings, a fused heterocyclyl could be bicyclic, tricyclic, tetracyclic, or polycyclic fused heterocyclyl. The group can be attached to the remainder of the molecule through either ring.

Specifically, the term “bicyclic fused heterocyclyl” refers to a 7 to 12-membered, preferably 7- to 10-membered, more preferably 9- or 10-membered fused heterocyclyl as defined herein comprising two fused rings and comprising 1 to 4 heteroatoms selected from nitrogen, oxygen or optionally oxidized sulfur as ring members. Typically, a bicyclic fused heterocyclyl is 5-membered/5-membered, 5-membered/6-membered. 6-membered/6-membered, or 6-membered/7-membered bicyclic fused heterocyclyl. Representative examples of (bicyclic) fused heterocycles include without limitation to the following groups: octahydrocyclopenta[c]pyrrole, octahydropyrrolo[3, 4-c]pyrrolyl, octahydroisoindolyl, isoindolinyl, octahydro-benzo[b][1, 4]dioxin, indolinyl, isoindolinyl, benzopyranyl, dihydrothiazolopyrimidinyl, tetrahydroquinolyl, tetrahydroisoquinolyl (or tetrahydroisoquinolinyl), dihydrobenzofuranyl, dihydrobenzoxazinyl, dihydrobenzoimidazolyl, tetrahydrobenzothienyl, tetrahydrobenzofuranyl, benzodioxolyl, benzodioxonyl, chromanyl, chromenyl, octahydrochromenyl, dihydrobenzodioxynyl, dihydrobenzoxczinyl, dihydrobenzodioxepinyl, dihydrothienodioxynyl, dihydrobenzooxazepinyl, tetrahydrobenzooxazepinyl, dihydrobenzoazepinyl, tetrahydrobenzoazepinyl, isochromanyl, chromanyl, or tetrahydropyrazolopyrimidinyl (e.g., 4, 5, 6, 7-tetrahydropyrazolo[1, 5-a]pyrimidin-3-yl).

The term a “benzo fused heterocyclyl” is a bicyclic fused heterocyclyl in which a monocyclic 4 to 9-membered heterocyclyl as defined herein (preferably 5- or 6-membered) fused to a benzene ring.

The term “bridged heterocyclyl” refers to a 5 to 14-membered polycyclic heterocyclic alkyl group, wherein every two rings in the system share two disconnected atoms, comprising one or more heteroatoms selected from nitrogen, oxygen or optionally oxidized sulfur as ring members, with the remaining ring members being carbon. One or more rings of a bridged heterocyclyl group may contain one or more double bonds, but none of the rings has a completely conjugated pi-electron system. Preferably, a bridged heterocyclyl is 6 to 14-membered, and more preferably 7 to 10-membered. According to the number of membered rings, a bridged heterocyclyl could be bicyclic, tricyclic, tetracyclic or polycyclic bridged heterocyclyl, and preferably refers to bicyclic, tricyclic or tetracyclic bridged heterocyclyl, and more preferably bicyclic or tricyclic bridged heterocyclyl. Representative examples of bridged heterocyclyls include without limitation to the following groups: 2-azabicyclo[2.2.1 ]heptyl, azabicyclo[3.1.0]hexyl, 2-azabicyclo[2.2.2 ]octyl and 2-azabicyclo[3.3.2]decyl.

The term “at least one substituent” disclosed herein includes, for example, from 1 to 4, such as from 1 to 3, further as 1 or 2, substituents, provided that the theory of valence is met. For example, “at least one substituent R^{6d}” disclosed herein includes from 1 to 4, such as from 1 to 3, further as 1 or 2, substituents selected from the list of R^{6d} as disclosed herein.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastercomers fall within the broader class of stereoisomers. All such possible stereoisomers as substantially pure resolved enantiomers, racemic mixtures thereof, as well as mixtures of diastercomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

The term “substantially pure” as used herein means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

“Diastercomers” refer to stereoisomers of a compound with two or more chiral centers but which are not mirror images of one another. Diastereomeric mixtures can be separated into their individual diastercomers on the basis of their physical or chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers and diastereomers can also be separated by the use of a chiral HPLC column.

A single stereoisomer, e.g., a substantially pure enantiomer, may be obtained by resolution of the racemic mixture using a method such as formation of diastercomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastercomeric salts with chiral compounds and separation by fractional crystallization or other methods, (2) formation of diastercomeric compounds with chiral derivatizing reagents, separation of the diastereomers, and conversion to the pure stercoisomers, and (3) separation of the substantially pure or enriched stereoisomers directly under chiral conditions. Sec: Wainer, Irving W., Ed. Drug Stereochemistry: Analytical Methods and Pharmacology. New York: Marcel Dekker, Inc., 1993.

“Pharmaceutically acceptable salts” refer to those salts which are, within the scope of sound medical judgment, suitable for use in contact with the tissues of humans and animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable basc.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” includes salts of at least one compound of Formula (I), and salts of the stereoisomers of the compound of Formula (I), such as salts of enantiomers, and/or salts of diastercomers.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic agent, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined herein, a disease or disorder in a subject. In the case of combination therapy, the “therapeutically effective amount” refers to the total amount of the combination objects for the effective treatment of a disease, a disorder or a condition.

The pharmaceutical composition comprising the compound disclosed herein can be administrated via oral, inhalation, rectal, parenteral or topical route to a subject in need thereof. For oral administration, the pharmaceutical composition may be a regular solid formulation such as tablets, powder, granule, capsules and the like, a liquid formulation such as water or oil suspension or other liquid formulation such as syrup, solution, suspension or the like; for parenteral administration, the pharmaceutical composition may be solution, water solution, oil suspension concentrate, lyophilized powder or the like. Preferably, the formulation of the pharmaceutical composition is selected from tablet, coated tablet, capsule, suppository, nasal spray or injection, more preferably tablet or capsule. The pharmaceutical composition can be a single unit administration with an accurate dosage. In addition, the pharmaceutical composition may further comprise additional active ingredients.

All formulations of the pharmaceutical composition disclosed herein can be produced by the conventional methods in the pharmaceutical field. For example, the active ingredient can be mixed with one or more excipients, then to make the desired formulation. The “pharmaceutically acceptable excipient” refers to conventional pharmaceutical carriers suitable for the desired pharmaceutical formulation, for example: a diluent, a vehicle such as water, various organic solvents, etc., a filler such as starch, sucrose, etc., a binder such as cellulose derivatives, alginates, gelatin and polyvinylpyrrolidone (PVP); a wetting agent such as glycerol; a disintegrating agent such as agar, calcium carbonate and sodium bicarbonate; an absorption enhancer such as quaternary ammonium compound; a surfactant such as hexadecanol; an absorption carrier such as Kaolin and soap clay; a lubricant such as talc, calcium stearate, magnesium stearate, polyethylene glycol, etc. In addition, the pharmaceutical composition further comprises other pharmaceutically acceptable excipients such as a decentralized agent, a stabilizer, a thickener, a complexing agent, a buffering agent, a permeation enhancer, a polymer, an aromatic, a sweetener, a dye and etc.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cₙ₋ₘ” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

### GENERAL REACTION SCHEME FOR COMPOUND PREPARATION

The subject compounds and pharmaceutically acceptable salts thereof, can be prepared from (a) commercially available starting materials (b) known starting materials which may be prepared as described in literature procedures (c) new intermediates described in the schemes and experimental procedures herein. In making the compounds of the invention, the order of synthetic steps may be varied to increase the yield of desired product. Some of compounds in this invention may be generated by the methods as shown in the following reaction schemes and the description thereof.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from room temperature to the solvent's boiling temperature. A given reaction can be carried out in one solvent or mixture of solvents.

The selection of appropriate protecting group, can be readily determined by one skilled in the art.

Reactions can be monitored according to any suitable method known in the art, such as NMR, UV, HPLC, LC-MS and TLC. Compounds can be purified by a variety of methods, including HPLC and normal phase silica chromatography.

The compounds disclosed herein can be prepared by following Scheme I.

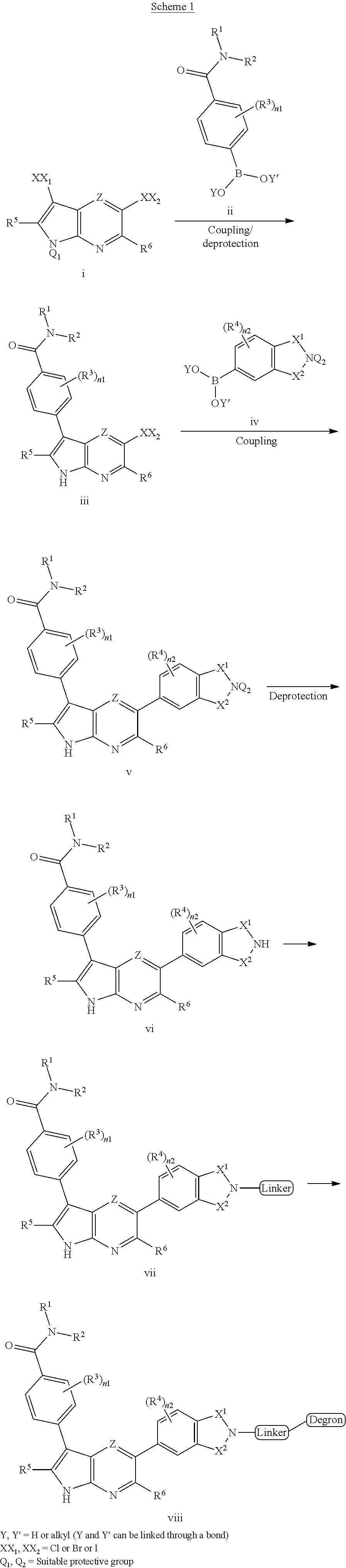

For example, compounds of Formula (I) can be synthesized as shown in Scheme I. Compound (i) can be reacted with compound (ii) using transition metal catalyzed reaction, followed by deprotection to give compound (iii); compound (iii) can be further reacted with compound (iv) using transition metal catalyzed reaction to give compound (v); compound (v) can be deprotected to give compound (vi); a linker can be attached to compound (vi) to give compound (vii); compound (vii) can be attached to a degron to give compound (viii) [i.e., Formula (I)].

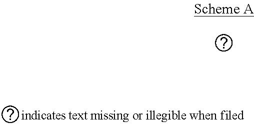

### EXAMPLES

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise. Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

### ABBREVIATIONS

In the following examples, the abbreviations below are used:

| | |
|---|---|
| Ac | Acetyl |
| ATP | Adenosine triphosphate |
| Boc | tert-butoxycarbonyl |
| BPD | Bis(pinacolato)diborane |
| DBU | 1,8-diazabicyclo [5.4.0]undec-7-ene |
| DCM | Dichloromethane |
| DMAP | 4-dimethylaminopyridine |
| DMF | N,N-dimethylformamide |
| DMSO | Dimethyl sulfoxide |
| dppf | 1,1′-ferrocenediyl-bis(diphenylphosphine) |
| EDCI | 1-Ethyl-3-(3-dimethylaminopropyl)carbodiimide |
| Et | Ethyl |
| FACS | Fluorescence-activated cell sorting |
| HATU | 1-[bis(dimethylamino)methylene]-1H-1,2,3- |
| | triazolo[4,5-b]pyridinium 3-oxid hexafluorophosphate |
| HOBT | Hydroxybenzotriazole |
| HPLC | High-performance liquid chromatography |
| Me | Methyl |
| MTBE | Methyl tert-butyl ether |
| NMR | Nuclear magnetic resonance |
| PE | Petroleum ether |
| Ph | Phenyl |
| T3P | Propanephosphonic acid anhydride |
| TBS | tert-butyl dimethyl silyl |
| TBST | Tris-buffered saline Tween |
| Titron B | Benzyltrimethylammonium hydroxide |
| TEA | Triethylamine |
| Tf | Trifluoromethanesulfonyl |
| TFA | Trifluoroacetic acid |
| THF | Tetrahydrofuran |
| TLC | Thin Layer Chromatography |
| TR-FRET | Time-resolved fluorescence-resonance energy transfer |

### Example 1: N-(2-(6-(7-(4-(dimethylcarbamoyl)-3-methylphenyl)-5H-pyrrolo[2,3-b]pyrazin-2-yl)-8-methyl-3,4-dihydroisoquinolin-2(1H)-yl)ethyl)-1-(2-(2,6-dioxopiperidin-3-yl)-1,3-dioxoisoindolin-5-yl)piperidine-4-carboxamide

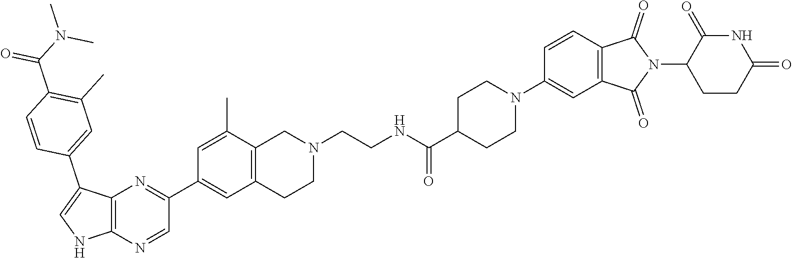 [SMILES: Cc1cc(-c2c[nH]c3ncc(-c4cc(C)c5c(c4)CCN(CCNC(=O)C4CCN(c6ccc7c(c6)C(=O)N(C6CCC(=O)NC6=O)C7=O)CC4)C5)nc23)ccc1C(=O)N(C)C]

### Step 1: 2-bromo-7-iodo-5-tosyl-5H-pyrrolo[2, 3-b]pyrazine

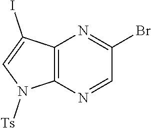 [SMILES: Cc1ccc(S(=O)(=O)n2cc(I)c3nc(Br)cnc32)cc1]

To a solution of 2-bromo-7-iodo-5H-pyrrolo[2, 3-b]pyrazine (162 g, 500 mmol) in anhydrous DMF (1500 mL) was added sodium hydride (30.0 g, 750 mmol) at 0° C. in portions. The resulting mixture was stirred for 15 min at 0° C., and then TsCl (124 g, 650 mmol) was added in portions. The mixture was warmed to room temperature while being stirred. After 3 h, the reaction mixture was poured into ice water (2 L) and the precipitate was collected by filtration. The solid was rinsed with water (200 mL×5) then dried under vacuum to give the title compound (239 g, 99%). LCMS (M+H)⁺=479.9.

### Step 2: 4-bromo-N,N,2-trimethylbenzamide

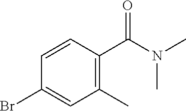

A mixture of 4-bromo-2-methylbenzoic acid (25.0 g, 116 mmol) in SOCl₂ (200 mL) was stirred at 60° C. for 3 h. The solvent was removed in vacuo. The residue was re-dissolved in anhydrous DCM (200 mL). Dimethylamine hydrochloride (14.0 g, 174.4 mmol) and TEA (80 mL, 581 mmol) was added at 0° C. The mixture was stirred at room temperature for 2 h. Water (200 mL) was added and the mixture was extracted with DCM (200 mL×3). The combined organic layer was washed with brine (150 mL), dried over Na₂SO₄, concentrated under reduced pressure to give the title compound (28.0 g, 99%). LC-MS (M+H)⁺=242.0, 244.0.

### Step 3: N,N,2-trimethyl-4-(4,4,5,5-tetramethyl-1,3,2-dioxaborolan-2-yl)benzamide

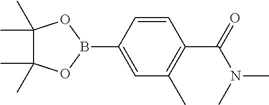

4-bromo-N,N,2-trimethylbenzamide (28.0 g, 115 mmol), BPD (44.0 g, 174 mmol), Pd(dppf)Cl₂ (5.1 g, 6.94 mmol) and AcOK (22.7 g, 231 mmol) was added to dioxane (400 mL) under nitrogen. The reaction mixture was heated to reflux overnight then cooled to room temperature. EtOAc (400 mL) was added and the mixture was washed with brine (300 mL×2). The aqueous layer was extracted with EtOAc (400 mL). The combined organic layer was dried over Na₂SO₄ then concentrated under reduced pressure. The residue was purified by silica gel column chromatography, eluted with EtOAc/PE (1:5 to 2:1) to give the title compound (26.0 g, 73%). LC-MS (M+H)⁺=290.1.

### Step 4: 4-(2-bromo-5-tosyl-5H-pyrrolo[2,3-b]pyrazin-7-yl)-N,N,2-trimethylbenzamide

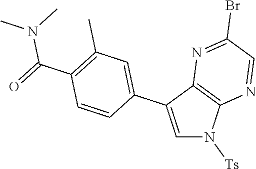 [SMILES: Cc1ccc(S(=O)(=O)n2cc(-c3ccc(C(=O)N(C)C)c(C)c3)c3nc(Br)cnc32)cc1]

To a solution of 2-bromo-7-iodo-5-tosyl-5H-pyrrolo[2,3-b]pyrazine (16.5 g, 34.6 mmol) and N,N,2-trimethyl-4-(4,4,5,5-tetramethyl-1,3,2-dioxaborolan-2-yl)benzamide (10.0 g, 34.5 mmol) in dioxane (150 mL) and water (50 mL) was added K₂CO₃ (9.55 g, 69.2 mmol) and Pd(dppf)Cl₂ (1.54 g, 2.07 mmol) under nitrogen atmosphere. After stirring for 5 h at 50° C., the reaction mixture was cooled to room temperature and concentrated under reduced pressure. The residue was purified by silica gel column chromatography, eluted with EtOAc/PE (1:2 to 2:1) to give the title compound (11.0 g, 62%). ¹H NMR (400 MHz, DMSO-d6) δ8.88 (s, 1H), 8.68 (s, 1H), 8.09-8.01 (m, 4H), 7.49-7.44 (m, 2H), 7.29 (d, J=7.8 Hz, 1H), 3.02 (s, 3H), 2.79 (s, 3H), 2.36 (s, 3H), 2.27 (s, 3H). LCMS (M+H)⁺=513.0.

### Step 5: tert-butyl (pivaloyloxy)carbamate

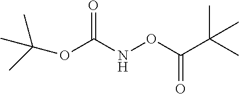

To a stirred solution of pivaloyl anhydride (17.0 g, 86.7 mmol) in CHCl₃ (300 mL) was added tert-butyl N-hydroxycarbamate (10.0 g, 71.3 mmol) dropwise at 0° C. The reaction mixture was heated to 70° C. for 16 h. The mixture was cooled down to room temperature and concentrated under vacuum. The residue was partitioned between EtOAc (500 mL) and water (500 mL). The organic layer was dried over Na₂SO₄, filtered and concentrated under vacuum to provide the title compound (7.2 g, 46%).

### Step 6: O-pivaloylhydroxylamine trifluoromethanesulfonate salt

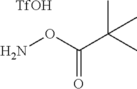

TfOH (18.9 g, 125.7 mmol) was added to a solution of tert-butyl (pivaloyloxy)carbamate (24.8 g, 114.3 mmol) in MTBE (230 mL) at 0° C. and stirred at room temperature for 4 h. The volume of solution was reduced to about 100 mL under reduced pressure and the precipitate was collected by filtration. The solid was dried under vacuum to give the title compound (26.0 g, 85%). LC-MS (M+H)⁺=118.0.

### Step 7: 4-bromo-2-methyl—N-(pivaloyloxy)benzamide

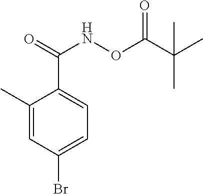 [SMILES: Cc1cc(Br)ccc1C(=O)NOC(=O)C(C)(C)C]

DIPEA (15.7 g, 121.6 mmol) was added to a solution of 4-bromo-2-methylbenzoic acid (8.82 g, 40.52 mmol) in THF (150 mL) at 0° C., followed by T3P (25.8 g, 81.1 mmol) and O-pivaloylhydroxylamine trifluoromethanesulfonate salt (26.0 g, 97.3 mmol). The reaction was stirred at room temperature overnight. Brine (100 mL) was added and the mixture was extracted with ethyl acetate (100 mL×3). The combined organic layer was dried over Na₂SO₄, filtered and concentrated under reduced pressure. The residue was purified by silica gel column chromatography to give the title compound (7.5 g, 59%). LC-MS (M+H)⁺=314.0.

Step 8: 6-bromo-8-methyl-3,4-dihydroisoquinolin-1(2H)-one

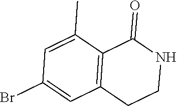

KOAc (5.16 g, 52.5 mmol) and dichloro(pentamethylcyclopentadienyl)rhodium (III) dimer (737.7 mg, 1.19 mmol) were added to a solution of 4-bromo-2-methyl-N-(pivaloyloxy)benzamide (7.5 g, 23.9 mmol) in acetonitrile (150 mL). The solution was stirred under an ethylene atmosphere (3 bar) at room temperature for overnight. The solvent was removed in vacuo and the residue was partitioned between water (20 mL) and ethyl acetate (50 mL). The organic layer was dried over Na₂SO₄, filtered and concentrated. The residue was purified by silica gel column chromatography to give the title compound (4.67 g, 82%). LC-MS (M+H)⁺=240.0.

### Step 9: 6-bromo-8-methyl-1,2,3,4-tetrahydroisoquinoline

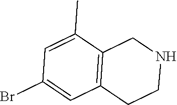

To 6-bromo-8-methyl-3,4-dihydroisoquinolin-1(2H)-one (4.67 g, 19.5 mmol) was added BH₃ in THF (1.0 M, 77.8 mL, 77.8 mmol) and the reaction mixture was refluxed overnight. The mixture was cooled to 0° C. and MeOH (5 mL) was added followed by HCl (2 M, 25 mL). The solution was heated to 80 ° C. for 3 h. The mixture was cooled to room temperature and solvent was removed in vacuo. The residue was dissolved in DCM (50 mL) and the solution was successively washed with saturated NaHCO₃ (30 mL) and brine (30 mL), dried over Na₂SO₄, filtered and concentrated. The residue was purified by silica gel column chromatography to give the title compound (3.79 g, 86%). LC-MS (M+H)⁺=226.0.

### Step 10: tert-butyl 6-bromo-8-methyl-3,4-dihydro-1H-isoquinoline-2-carboxylate

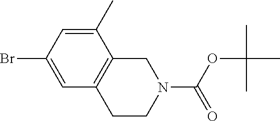 [SMILES: Cc1cc(Br)cc2c1CN(C(=O)OC(C)(C)C)CC2]

At 0 ° C., to a solution of 6-bromo-8-methyl-1,2,3,4-tetrahydroisoquinoline hydrochloride (36.0 g, 137 mmol) in DCM (400 mL) was added triethylamine (34.7 g, 343 mmol), DMAP (1.67 g, 13.7 mmol) and Boc₂O (74.8 g, 342.1 mmol) in portions. The mixture was warmed to room temperature and stirred for 16 h. The mixture was concentrated under vacuum. The residue was purified by silica gel chromatography, eluting with EtOAc in PE (0% to 50% gradient) to give the title compound (23.3 g, 52%). LC-MS (M-t-Bu+H)⁺=269.9.

### Step 11: tert-butyl 8-methyl-6-(4,4,5,5-tetramethyl-1,3,2-dioxaborolan-2-yl)-3,4-dihydro-1H-isoquinoline-2-carboxylate

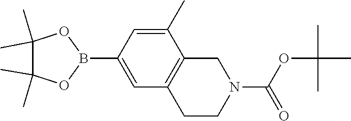 [SMILES: Cc1cc(B2OC(C)(C)C(C)(C)O2)cc2c1CN(C(=O)OC(C)(C)C)CC2]

To a solution of tert-butyl 6-bromo-8-methyl-3,4-dihydro-1H-isoquinoline-2-carboxylate (20.0 g, 61.3 mmol) in dioxane (500 mL) was added BPD (23.4 g, 92.1 mmol), KOAc (18.0 g, 183.9 mmol) and Pd(dppf)Cl₂·CH₂Cl2 (5.0g, 6.1 mmol) under nitrogen. The reaction was heated to 100° C. and stirred for 3 h. The mixture was cooled to room temperature and concentrated under reduced pressure. The residue was purified by silica gel chromatography, eluting with EtOAc in PE (0% to 50% gradient) to give the title compound (19.0 g, 83%). LC-MS (M-Boc+H)⁺=274.1.

### Step 12: tert-butyl 6-(7-(4-(dimethylcarbamoyl)-3-methylphenyl)-5-tosyl-5H-pyrrolo[2,3-b]pyrazin-2-yl)-8-methyl-3,4-dihydroisoquinoline-2(1H)-carboxylate

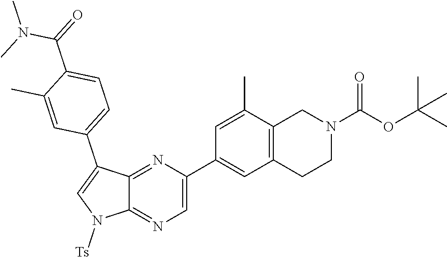 [SMILES: Cc1ccc(S(=O)(=O)n2cc(-c3ccc(C(=O)N(C)C)c(C)c3)c3nc(-c4cc(C)c5c(c4)CCN(C(=O)OC(C)(C)C)C5)cnc32)cc1]

To a solution of 4-(2-bromo-5-tosyl-5H-pyrrolo[2,3-b]pyrazin-7-yl)-N,N,2-trimethylbenzamide (20.0 g, 39 mmol) in dioxane (300 mL) and H₂O (30 mL) was added tert-butyl 8-methyl-6-(4,4,5,5-tetramethyl-1,3,2-dioxaborolan-2-yl)-3,4-dihydro-1H-isoquinoline-2-carboxylate (20.0 g, 51 mmol), K₂CO₃ (10.8 g, 78 mmol) and Pd(dppf)C12.CH₂Cl2 (3.20 g, 3.9 mmol) under nitrogen. The mixture was heated to 100° C. and stirred for 2 h. The mixture was cooled to room temperature and concentrated under reduced pressure. The residue was purified by silica gel chromatography, eluting with MeOH in DCM (0% to 10% gradient) to give the title compound (11.8 g, 45%). LC-MS (M+H)⁺=680.4.

Step 13: tert-butyl 6-(7-(4-(dimethylcarbamoyl)-3-methylphenyl)-5H-pyrrolo[2,3-b]pyrazin-2-yl)-8-methyl-3,4-dihydroisoquinoline-2(1H)-carboxylate

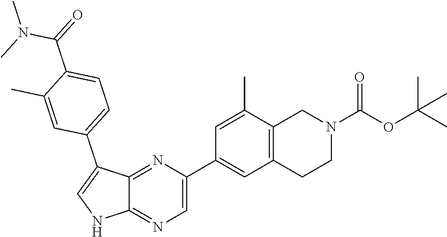 [SMILES: Cc1cc(-c2c[nH]c3ncc(-c4cc(C)c5c(c4)CCN(C(=O)OC(C)(C)C)C5)nc23)ccc1C(=O)N(C)C]

To a solution of tert-butyl 6-(7-(4-(dimethylcarbamoyl)-3-methylphenyl)-5-tosyl-5H-pyrrolo[2,3-b]pyrazin-2-yl)-8-methyl-3,4-dihydroisoquinoline-2(1H)-carboxylate (11.8 g, 18.7 mmol) in MeOH (400 mL) was added aqueous NaOH (2.0 M, 37 mL, 74 mmol) at room temperature. The mixture was heated to 60° C. and stirred for 20 min. The mixture was cooled to room temperature and concentrated under vacuum. The residue was purified by silica gel chromatography, eluting with MeOH in DCM (0% to 10% gradient) to give the title compound (6.3 g, 64%). LC-MS (M+H)⁺=526.3.

### Step 14: N,N,2-trimethyl-4-(2-(8-methyl-1,2,3,4-tetrahydroisoquinolin-6-yl)-5H-pyrrolo[2,3-b]pyrazin-7-yl)benzamide hydrochloride

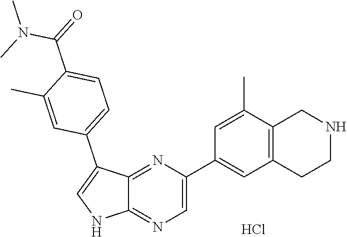 [SMILES: Cc1cc(-c2c[nH]c3ncc(-c4cc(C)c5c(c4)CCNC5)nc23)ccc1C(=O)N(C)C ; Cl]

Tert-butyl 6-(7-(4-(dimethylcarbamoyl)-3-methylphenyl)-5H-pyrrolo[2,3-b]pyrazin-2-yl)-8-methyl-3,4-dihydroisoquinoline-2(1H)-carboxylate (6.3 g, 12.2 mmol) was slowly added into HCI in dioxane (4.0 M, 100 mL, 0.40 mol) at room temperature. The mixture was stirred for 3 h then concentrated under vacuum. The residue was triturated with ether (200 mL) and the solid was collected by filtration then washed with ether (100 mL×3) to give the title compound (5.47 g, 97%). ¹H NMR (300 MHz, DMSO-d₆) δ12.44 (s, 1H), 9.76 (s, 2H), 8.91 (s, 1H), 8.48 (d, J=3.0 Hz, 1H), 8.28-8.13 (m, 2H), 7.93 (d, J=14.1 Hz, 2H), 7.23 (d, J=8.1, 2.9 Hz, 1H), 4.22 (s, 2H), 3.42-3.32 (m, 2H), 3.15 (t, J=5.9 Hz, 2H), 3.02 (s, 3H), 2.82 (s, 3H), 2.30 (d, J=11.7, 3.0 Hz, 6H). LC-MS (M+H)⁺=426.3.

### Step 15: tert-butyl (2-bromoethyl)carbamate

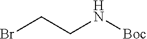 [SMILES: CC(C)(C)OC(=O)NCCBr]

A solution of 2-bromoethan-1-amine hydrochloride (4.0 g, 19.6 mmol), (Boc)₂O (4.7 g, 21.6 mmol) and triethylamine (13 mL, 98.0 mmol) in THF (80 mL) was stirred at room temperature for overnight. The mixture was then partitioned between water (80 mL) and EtOAc (80 mL). The organic layer was washed with brine (80 mL), dried over Na₂SO₄ and filtered. The crude was purified by silica gel column chromatography, eluting with PE/EtOAc=10:1 to give the title compound (3.7 g, 84%). LC-MS (M+H)⁺=224.0.

Step 16: tert-butyl (2-(6-(7-(4-(dimethylcarbamoyl)-3-methylphenyl)-5H-pyrrolo[2,3-b]pyrazin-2-yl)-8-methyl-3,4-dihydroisoquinolin-2(1H)-yl)ethyl)carbamate

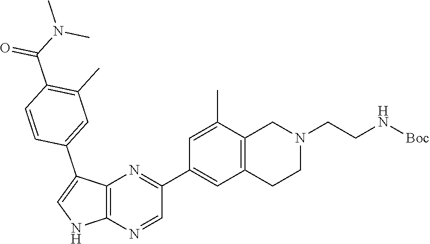 [SMILES: Cc1cc(-c2c[nH]c3ncc(-c4cc(C)c5c(c4)CCN(CCNC(=O)OC(C)(C)C)C5)nc23)ccc1C(=O)N(C)C]

A mixture of N,N,2-trimethyl-4-(2-(8-methyl-1,2,3,4-tetrahydroisoquinolin-6-yl)-5H-pyrrolo[2,3-b]pyrazin-7-yl)benzamide hydrochloride (1.0 g, 2.2 mmol), tert-butyl (2-bromoethyl)carbamate (580 mg, 2.6 mmol) and Na₂CO₃ (460 mg, 4.3 mmol) in DMF (15 mL) was stirred at room temperature for overnight. The mixture was diluted with water (60 mL) and then extracted with EtOAc (50 mL). The organic layer was dried over Na₂SO₄, filtered and concentrated in vacuo. The crude was purified by silica gel prep-TLC, developing with DCM/MeOH=10:1 to give the title compound (400 mg, 33%). LC-MS (M+H)⁺=569.3.

### Step 17: 4-(2-(2-(2-aminoethyl)-8-methyl-1,2,3,4-tetrahydroisoquinolin-6-yl)-5H-pyrrolo[2,3-b]pyrazin-7-yl)-N,N,2-trimethylbenzamide dihydrochloride

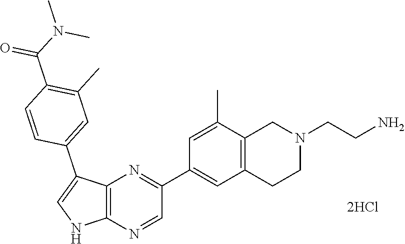 [SMILES: Cc1cc(-c2c[nH]c3ncc(-c4cc(C)c5c(c4)CCN(CCN)C5)nc23)ccc1C(=O)N(C)C]

To tert-butyl (2-(6-(7-(4-(dimethylcarbamoyl)-3-methylphenyl)-5H-pyrrolo[2,3-b]pyrazin-2-yl)-8-methyl-3,4-dihydroisoquinolin-2(1H)-yl)ethyl)carbamate (400 mg, 0.79 mmol) was added HCl in dioxane (4.0 M, 10 mL). The reaction mixture was stirred at room temperature overnight. The precipitation was collected by filtration and washed with MTBE (2 x 10 mL) to give the title compound (400 mg, 96%). LC-MS (M+H)⁺=469.6.

### step 18: N-(2-(6-(7-(4-(dimethylcarbamoyl)-3-methylphenyl)-5H-pyrrolo[2,3-b]pyrazin-2-yl)-8-methyl-3,4-dihydroisoquinolin-2(1H)-yl)ethyl)-1-(2-(2,6-dioxopiperidin-3-yl)-1,3-dioxoisoindolin-5-yl)piperidine-4-carboxamide

To a solution of 4-(2-(2-(2-aminoethyl)-8-methyl-1,2,3,4-tetrahydroisoquinolin-6-yl)-5H-pyrrolo[2,3-b]pyrazin-7-yl)-N,N,2-trimethylbenzamide dihydrochloride (100 mg, 0.18 mmol), 1-(2-(2,6-dioxopiperidin-3-yl)-1,3-dioxoisoindolin-5-yl)piperidine-4-carboxylic acid (84 mg, 0.22 mmol) and HATU (79 mg, 0.21 mmol) in DMF (3 mL) was added triethylamine (0.14 mL, 1.0 mmol). The mixture was stirred at room temperature for overnight. The mixture was diluted with water (20 mL), extracted with EtOAc (20 mL). The organic layer was dried over Na₂SO₄, filtered and concentrated in vacuo. The crude was purified by silica gel prep-TLC, developing with DCM/MeOH=8:1 to give Example 1 (10 mg, 7%). ¹H NMR (400 MHz, DMSO) δ12.28 (d, J=2.7 Hz, 1H), 11.02 (s, 1H), 8.82 (s, 1H), 8.40 (d, J=2.6 Hz, 1H), 8.20 (s, 1H), 8.13 (d, J=8.1 Hz, 1H), 7.82-7.76 (m, 2H), 7.74 (s, 1H), 7.58 (d, J =8.5 Hz, 1H), 7.26 (s, 1H), 7.17 (d, J=8.1 Hz, 2H), 5.02-4.97 (m, 1H), 4.01-3.98 (m, 2H), 3.51 (s, 2H), 3.25 (s, 3H), 2.96-2.93 (m, 5H), 2.91-2.82 (m, 3H), 2.76 (s, 3H), 2.66 (d, J=5.1 Hz, 2H), 2.60-2.52 (m, 3H), 2.22 (s, 6H), 1.98-1.89 (m, 1H), 1.70 (d, J=10.5 Hz, 2H), 1.59-1.51 (m, 2H). LC-MS (M+H)⁺=836.9.

### Example 2: 4-(2-(2-(3-(((1-(4-(2,6-dioxopiperidin-3-yl)phenyl)piperidin-4-yl)methyl)amino)-3-oxopropyl)-8-methyl-1,2,3,4-tetrahydroisoquinolin-6- yl)-5H-pyrrolo[2,3-b]pyrazin-7-yl)-N,N,2-trimethylbenzamide

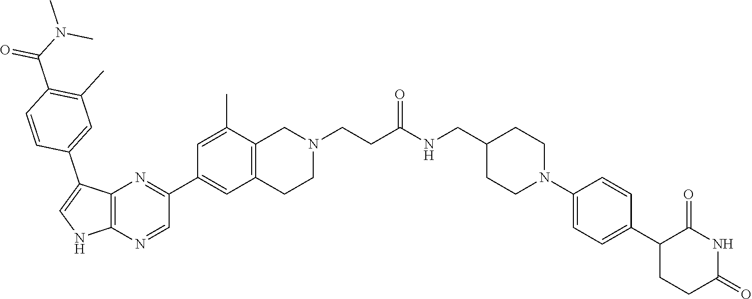 [SMILES: Cc1cc(-c2c[nH]c3ncc(-c4cc(C)c5c(c4)CCN(CCC(=O)NCC4CCN(c6ccc(C7CCC(=O)NC7=O)cc6)CC4)C5)nc23)ccc1C(=O)N(C)C]

### Step 1: tert-butyl 3-(6-(7-(4-(dimethylcarbamoyl)-3-methylphenyl)-5H-pyrrolo[2,3-b]pyrazin-2-yl)-8-methyl-3,4-dihydroisoquinolin-2(1H)-yl)propanoate

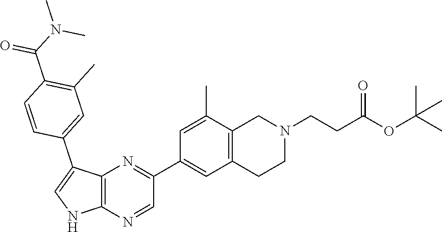 [SMILES: Cc1cc(-c2c[nH]c3ncc(-c4cc(C)c5c(c4)CCN(CCC(=O)OC(C)(C)C)C5)nc23)ccc1C(=O)N(C)C]

To a mixture of N,N,2-trimethyl-4-(2-(8-methyl-1,2,3,4-tetrahydroisoquinolin-6-yl)-5H-pyrrolo[2,3-b]pyrazin-7-yl)benzamide (1.0 g, 0.22 mmol) and tert-butyl 3-bromopropanoate (500 mg, 0.24 mmol) in DMF (15 mL) was added K₂CO₃ (0.60 g, 0.65 mmol). The mixture was stirred at room temperature for overnight then diluted with water (50 mL). The mixture was extracted with EtOAc (30 mL). The organic layer was dried over Na₂SO₄, filtered and concentrated in vacuo. The crude was purified by silica gel prep-TLC, developing with DCM/MeOH=10:1 to give the title compound (700 mg, 53%). LC-MS (M+H)⁺=554.3.

### Step 2: 3-(6-(7-(4-(dimethylcarbamoyl)-3-methylphenyl)-5H-pyrrolo[2,3-b]pyrazin-2-yl)-8-methyl-3,4-dihydroisoquinolin-2(1H)-yl)propanoic acid hydrochloride

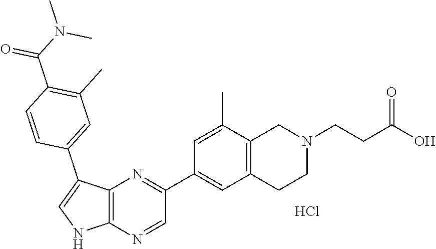 [SMILES: Cc1cc(-c2c[nH]c3ncc(-c4cc(C)c5c(c4)CCN(CCC(=O)O)C5)nc23)ccc1C(=O)N(C)C ; Cl]

To tert-butyl 3-(6-(7-(4-(dimethylcarbamoyl)-3-methylphenyl)-5H-pyrrolo[2,3-b]pyrazin-2-yl)-8-methyl-3,4-dihydroisoquinolin-2(1H)-yl)propanoate (700 mg, 1.4 mmol) was added HCl in dioxane (4.0 M, 10 mL). The mixture was stirred at room temperature for overnight. The precipitate was collected by filtration and washed with MTBE (2×10 mL) to give the title compound (650 mg, 87%). LC-MS (M+H)⁺=498.6.

### Step 3: 4-(2-(2-(3-(((1-(4-(2,6-dioxopiperidin-3-yl)phenyl)piperidin-4-yl)methyl)amino)-3-oxopropyl)-8-methyl-1,2,3,4-tetrahydroisoquinolin-6-yl)-5H-pyrrolo[2,3-b]pyrazin-7-yl)-N,N,2-trimethylbenzamide

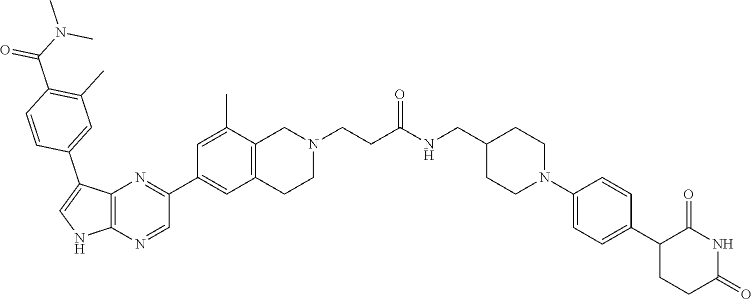 [SMILES: Cc1cc(-c2c[nH]c3ncc(-c4cc(C)c5c(c4)CCN(CCC(=O)NCC4CCN(c6ccc(C7CCC(=O)NC7=O)cc6)CC4)C5)nc23)ccc1C(=O)N(C)C]

### Example 2 (4 mg, 3%) was prepared in a manner similar to that in Example 1 Step 18 from 3-(4-(4-(aminomethyl)piperidin-1-yl)phenyl)piperidine-2,6-dione and 3-(6-(7-(4-(dimethylcarbamoyl)-3-methylphenyl)-5H-pyrrolo[2,3-b]pyrazin-2-yl)-8-methyl-3,4-dihydroisoquinolin-2(1H)-yl)propanoic acid hydrochloride (4 mg, 3%). ¹H NMR (400 MHz, DMSO) δ12.40 (s, 1H), 10.79 (s, 1H), 8.97 (s, 1H), 8.53 (s, 1H), 8.34 (s, 1H), 8.26 (d, J=7.8 Hz, 1H), 8.10 (s, 1H), 7.96 (s, 1H), 7.91 (s, 1H), 7.29 (d, J=8.0 Hz, 1H), 6.83 (d, J=8.5 Hz, 2H), 6.75 (d, J=8.7 Hz, 2H), 3.66-3.57 (m, 3H), 3.50-3.47 (m, 2H), 3.38 (s, 3H), 3.08 (s, 3H), 3.05-3.00 (m, 4H), 2.90-2.84 (m, 5H), 2.78 (s, 2H), 2.49-2.42 (m, 4H), 2.36-2.34 (m, 6H), 1.97-1.89 (m, 2H), 1.75-1.72 (m, 2H), 1.24-1.10 (m, 2H). LC-MS (M+H)⁺=781.9.

### Example 3: 4-(2-(2-(3-(4-(4-(2,4-dioxotetrahydropyrimidin-1(2H)-yl)phenyl)piperazin-1-yl)-3-oxopropyl)-8-methyl-1,2,3,4-tetrahydroisoquinolin-6-yl)-5H-pyrrolo[2,3-b]pyrazin-7-yl)-N,N,2-trimethylbenzamide

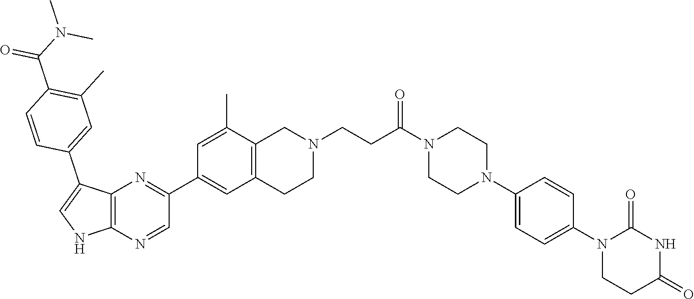 [SMILES: Cc1cc(-c2c[nH]c3ncc(-c4cc(C)c5c(c4)CCN(CCC(=O)N4CCN(c6ccc(N7CCC(=O)NC7=O)cc6)CC4)C5)nc23)ccc1C(=O)N(C)C]

Step 1: tert-butyl 4-(4-((3-ethoxy-3-oxopropyl)amino)phenyl)piperazine-1-carboxylate

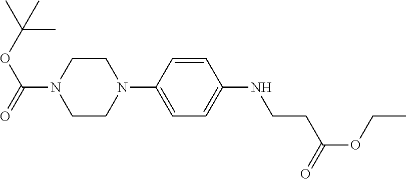 [SMILES: CCOC(=O)CCNc1ccc(N2CCN(C(=O)OC(C)(C)C)CC2)cc1]

A mixture of tert-butyl 4-(4-aminophenyl)piperazine-1-carboxylate (8.0 g, 28.2 mmol), DBU mono lactate (5.59 g, 23.1 mmol) and ethyl acrylate (4.33 g, 43.7 mmol) was stirred at 90° C. for 3 h. The mixture was cooled to room temperature and partitioned between EtOAc (40 mL) and water (40 mL). The organic layer was separated, dried over Na₂SO₄, filtered and concentrated under reduced pressure to give the title compound (8.5 g, 78%). LC-MS (M+H)⁺=378.2.

Step 2: tert-butyl 4-(4-(N-(3-ethoxy-3-oxopropyl)cyanamido)phenyl)piperazine-1-carboxylate

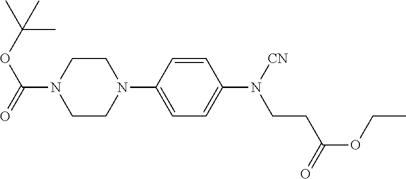 [SMILES: CCOC(=O)CCN(C#N)c1ccc(N2CCN(C(=O)OC(C)(C)C)CC2)cc1]

To a mixture of tert-butyl 4-(4-((3-ethoxy-3-oxopropyl)amino)phenyl)piperazine-1-carboxylate (8.5 g, 22.5 mmol) in toluene (5 mL) was added cyanogen bromide (2.8 g, 27.0 mmol) and NaHCO₃ (2.84 g, 33.8 mmol). The mixture was stirred for 3 h at room temperature then diluted with EtOAc (20 mL). The organic layer was washed with water (20 mL), dried over Na₂SO₄, filtered and concentrated under vacuum. The crude was purified by silica gel column chromatography, eluting with PE/EtOAc=10:1 to give the title compound (9.0 g, 95%). LC-MS (M+H)⁺=403.2.

### Step 3: tert-butyl 4-(4-(1-(3-ethoxy-3-oxopropyl)ureido)phenyl)piperazine-1-carboxylate

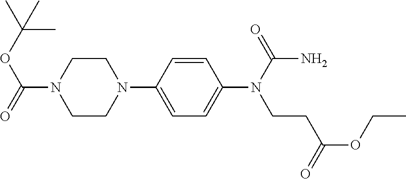 [SMILES: CCOC(=O)CCN(C(N)=O)c1ccc(N2CCN(C(=O)OC(C)(C)C)CC2)cc1]

A mixture of tert-butyl 4-(4-(N-(3-ethoxy-3-oxopropyl)cyanamido)phenyl)piperazine-1-carboxylate (1.0 g, 2.5 mmol), acetaldehyde oxime (0.93 g, 7.5 mmol) and InCl₃ (165 mg, 0.75 mmol) in toluene (10 mL) was heated to reflux for 1 h. The mixture was cooled to room temperature, partitioned between water (20 mL) and EtOAc (20 mL). The organic layer was separated, dried over Na₂SO₄, filtered and concentrated under vacuum to give the title compound (830 mg, 83%). LC-MS (M+H)⁺=421.2.

### Step 4: tert-butyl 4-(4-(2,4-dioxotetrahydropyrimidin-1(2H)-yl)phenyl)piperazine-1-carboxylate

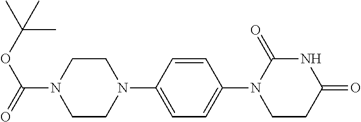 [SMILES: CC(C)(C)OC(=O)N1CCN(c2ccc(N3CCC(=O)NC3=O)cc2)CC1]

A mixture of tert-butyl 4-(4-(1-(3-ethoxy-3-oxopropyl)ureido)phenyl)piperazine-1-carboxylate (7.5 g, 17.9 mmol) and Titron B 40% (11.2 g, 26.8 mmol) in CH₃CN (30 mL) was heated to 60° C. for 10 min. The mixture was cooled to room temperature and concentrated under reduced pressure. The crude was purified by silica gel column chromatography, eluting with DCM/MeOH=50:1 to give the title compound (4.0 g, 58%). LC-MS (M+H)⁺=375.2.

### step 5: 1-(4-(piperazin-1-yl)phenyl)dihydropyrimidine-2,4(1H,3H)-dione hydrochloride

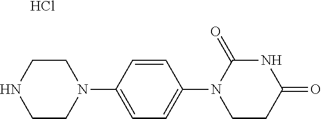 [SMILES: Cl ; O=C1CCN(c2ccc(N3CCNCC3)cc2)C(=O)N1]

To a suspension of tert-butyl 4-(4-(2,4-dioxotetrahydropyrimidin-1(2H)-yl)phenyl)piperazine-1-carboxylate (930 mg, 2.4 mmol) in dioxane (10 mL) was added HCl in dioxane (4.0 M, 20 mL). After 4 h, solid was filtered and washed with MTBE, dried under vacuum to give the title compound (770 mg, 100%). LC-MS (M+H)⁺=275.2.

### Step 6: 4-(2-(2-(3-(4-(4-(2,4-dioxotetrahydropyrimidin-1(2H)-yl)phenyl)piperazin-1-yl)-3-oxopropyl)-8-methyl-1,2,3,4-tetrahydroisoquinolin-6-yl)-5H-pyrrolo[2,3-b]pyrazin-7-yl)-N,N,2-trimethylbenzamide

A solution of 1-(4-(piperazin-1-yl)phenyl)dihydropyrimidine-2,4(1H,3H)-dione hydrochloride (57 mg, 0.18 mmol), 3-(6-(7-(4-(dimethylcarbamoyl)-3-methylphenyl)-5H-pyrrolo[2,3-b]pyrazin-2-yl)-8-methyl-3,4-dihydroisoquinolin-2(1H)-yl)propanoic acid hydrochloride (90 mg, 0.17 mmol), HATU (70 mg, 0.18 mmol) and triethylamine (0.12 mL, 0.84 mmol) in DMF (3 mL) was stirred at room temperature for overnight. Water (15 mL) was added and the mixture was extracted with EtOAc (30 mL). The organic layer was separated, dried over Na₂SO₄, filtered and concentrated under vacuum. The crude was purified by prep-TLC, developing with DCM/MeOH=8:1 to give Example 3 (4 mg, 3%). ¹H NMR (400 MHz, DMSO) δ12.35 (s, 1H), 10.28 (s, 1H), 8.89 (s, 1H), 8.47 (d, J=2.7 Hz, 1H), 8.26 (s, 1H), 8.20 (d, J=8.1 Hz, 1H), 7.89-7.75 (m, 2H), 7.24 (d, J=7.8 Hz, 1H), 7.17 (d, J=8.8 Hz, 2H), 6.96 (d, J=8.9 Hz, 2H), 3.77-3.52 (m, 5H), 3.32 (s, 6H), 3.20-3.13 (m, 2H), 3.13-3.07 (m, 2H), 3.03 (s, 3H), 2.97-2.90 (m, 2H), 2.83 (s, 3H), 2.77-2.70 (m, 2H), 2.69-2.63 (m, 2H), 2.33-2.20 (dm, 6H). LC-MS (M+H)⁺=754.4.

### Example 4: 4-(2-(2-(3-((1-(4-(2,4-dioxotetrahydropyrimidin-1(2H)-yl)phenyl)piperidin-4-yl)amino)-3-oxopropyl)-8-methyl-1,2,3,4- tetrahydroisoquinolin-6-yl)-5H-pyrrolo[2,3-b]pyrazin-7-yl)-N,N,2-trimethylbenzamide

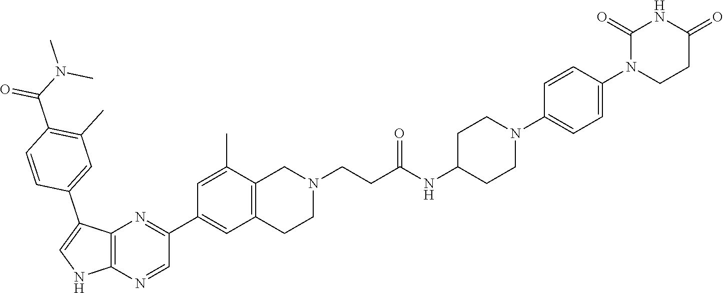 [SMILES: Cc1cc(-c2c[nH]c3ncc(-c4cc(C)c5c(c4)CCN(CCC(=O)NC4CCN(c6ccc(N7CCC(=O)NC7=O)cc6)CC4)C5)nc23)ccc1C(=O)N(C)C]

Example 4 was prepared in a manner similar to that in Example 3 step 6 from 3-(6-(7-(4-(dimethylcarbamoyl)-3-methylphenyl)-5H-pyrrolo[2,3-b]pyrazin-2-yl)-8-methyl-3,4-dihydroisoquinolin-2(1H)-yl)propanoic acid hydrochloride and 1-(4-(4-aminopiperidin-1-yl)phenyl)dihydropyrimidine-2,4(1H,3H)-dione (5 mg, 3%). ¹H NMR (400 MHz, DMSO) δ12.30 (s, 1H), 10.19 (s, 1H), 8.83 (s, 1H), 8.41 (s, 1H), 8.19 (s, 1H), 8.13 (d, J=8.1 Hz, 1H), 7.96 (s, 1H), 7.78 (s, 2H), 7.17 (d, J=7.5 Hz, 1H), 7.05 (d, J=7.8 Hz, 2H), 6.85 (d, J=9.0 Hz, 2H), 3.65-3.58 (m, 2H), 3.55-3.45 (m, 3H), 3.25 (s, 3H), 2.96 (s, 3H), 2.91-2.82 (m, 2H), 2.80-2.73 (m, 5H), 2.65-2.55 (m, 3H), 2.25-2.15 (m, 8H), 1.80-1.72 (m, 3H), 1.48-1.35 (m, 3H). LC-MS (M+H)⁺=768.3.

### Example 5: 4-(2-(2-(3-(4-(4-(2,4-dioxotetrahydropyrimidin-1(2H)-yl)phenyl)piperazin-1-yl)-3-oxopropyl)-8-methyl-1,2,3,4-tetrahydroisoquinolin-6-yl)-5H-pyrrolo[2,3-b]pyrazin-7-yl)-N,N-dimethylbenzamide

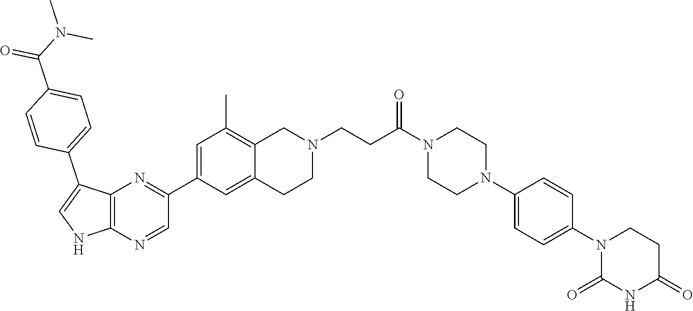 [SMILES: Cc1cc(-c2cnc3[nH]cc(-c4ccc(C(=O)N(C)C)cc4)c3n2)cc2c1CN(CCC(=O)N1CCN(c3ccc(N4CCC(=O)NC4=O)cc3)CC1)CC2]

### Step 1: 4-(2-bromo-5-tosyl-5H-pyrrolo[2,3-b]pyrazin-7-yl)-N,N-dimethylbenzamide

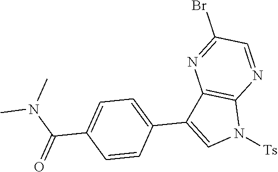 [SMILES: Cc1ccc(S(=O)(=O)n2cc(-c3ccc(C(=O)N(C)C)cc3)c3nc(Br)cnc32)cc1]

To a solution of 2-bromo-7-iodo-5-tosyl-5H-pyrrolo[2,3-b]pyrazine (4.77 g, 10 mmol) in dioxane (50 mL) and water (5 mL) was added (4-(dimethylcarbamoyl)phenyl)boronic acid (1.93 g, 10 mmol), K₂CO₃ (2.76 g, 20 mmol) and Pd(dppf)Cl₂·CH₂Cl₂ (818 mg, 1.0 mmol), then stirred for 16 h at 90° C. under N2 atmosphere. The mixture was cooled to room temperature, diluted with EtOAc (100 mL), then successively washed with H₂O (60 mL), brine (60 mL), dried over Na₂SO₄, filtered and concentrated under vacuum. The residue was purified by silica gel chromatograph with PE: EtOAc=1:3 to give the title compound (2.20 g, 44%). LC-MS (M+H)⁺=499.1, 501.1.

### Step 2: tert-butyl 6-(7-(4-(dimethylcarbamoyl)phenyl)-5-tosyl-5H-pyrrolo[2,3-b]pyrazin-2-yl)-8-methyl-3,4-dihydroisoquinoline-2(1H)-carboxylate

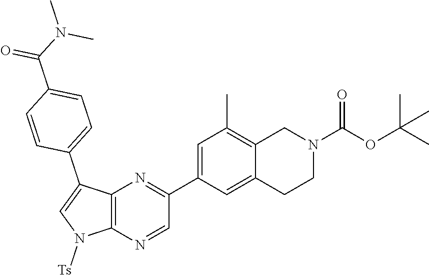 [SMILES: Cc1ccc(S(=O)(=O)n2cc(-c3ccc(C(=O)N(C)C)cc3)c3nc(-c4cc(C)c5c(c4)CCN(C(=O)OC(C)(C)C)C5)cnc32)cc1]

To a solution of 4-(2-bromo-5-tosyl-5H-pyrrolo[2,3-b]pyrazin-7-yl)-N,N-dimethylbenzamide (996 mg, 2 mmol) in dioxane (10 mL) and water (1 mL) was added tert-butyl 8-methyl-6-(4,4,5,5-tetramethyl-1,3,2-dioxaborolan-2-yl)-3,4-dihydro-1H-isoquinoline-2-carboxylate (746 mg, 2 mmol), K₂CO₃ (828 mg, 6 mmol) and Pd(dppf)Cl₂ CH₂Cl₂ (164 mg, 0.2 mmol), then the mixture was stirred for 3 h at 100° C. under N₂. The mixture cooled to room temperature, diluted with EtOAc (30 mL), successively washed with H₂O (20 mL), brine (20 mL), dried over Na₂SO₄, filtered and concentrated under vacuum. The residue was purified by silica gel chromatograph with EtOAc to give the title compound (1.1 g, 83%). LC-MS (M+H)⁺=666.4.

### Step 3: N,N-dimethyl-4-(2-(8-methyl-1,2,3,4-tetrahydroisoquinolin-6-yl)-5-tosyl-5H-pyrrolo[2,3-b]pyrazin-7-yl)benzamide hydrochloride

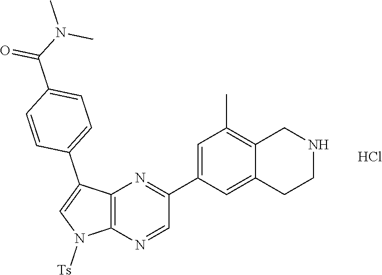 [SMILES: Cc1ccc(S(=O)(=O)n2cc(-c3ccc(C(=O)N(C)C)cc3)c3nc(-c4cc(C)c5c(c4)CCNC5)cnc32)cc1 ; Cl]

To a solution of tert-butyl 6-(7-(4-(dimethylcarbamoyl)phenyl)-5-tosyl-5H-pyrrolo[2,3-b]pyrazin-2-yl)-8-methyl-3,4-dihydroisoquinoline-2(1H)-carboxylate (1.1 g, 1.9 mmol) in dioxane (2 mL) was added HCl/dioxane (4 M, 10 mL) dropwise at 0° C. The reaction mixture was stirred for 1 h at room temperature and then concentrated under vacuum to give the title compound (0.99 g, 99%). LC-MS (M+H)⁺=566.3.

### Step 4: tert-butyl 3-(6-(7-(4-(dimethylcarbamoyl)phenyl)-5-tosyl-5H-pyrrolo[2,3-b]pyrazin-2-yl)-8-methyl-3,4-dihydroisoquinolin-2(1H)-yl)propanoate

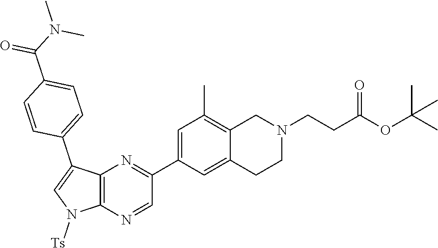 [SMILES: Cc1ccc(S(=O)(=O)n2cc(-c3ccc(C(=O)N(C)C)cc3)c3nc(-c4cc(C)c5c(c4)CCN(CCC(=O)OC(C)(C)C)C5)cnc32)cc1]

To a solution of N,N-dimethyl-4-(2-(8-methyl-1,2,3,4-tetrahydroisoquinolin-6-yl)-5-tosyl-5H-pyrrolo[2,3-b]pyrazin-7-yl)benzamide hydrochloride (678 mg, 1.13 mmol) in DMF (5 mL) was added tert-butyl 3-bromopropanoate (1.25 g, 6.0 mmol). The reaction mixture was stirred for overnight at 40° C., diluted with EtOAc (30 mL), successively washed with brine (2×15 mL), dried over Na₂SO₄, filtered and concentrated under vacuum. The residue was purified by prep-TLC with MeOH: DCM=1:25 to give the title compound (300 mg, 38%). LC-MS (M+H)⁺=694.4.

### Step 5: tert-butyl 3-(6-(7-(4-(dimethylcarbamoyl)phenyl)-5H-pyrrolo[2,3-b]pyrazin-2-yl)-8-methyl-3,4-dihydroisoquinolin-2(1H)-yl)propanoate

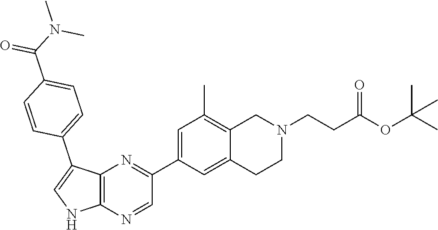 [SMILES: Cc1cc(-c2cnc3[nH]cc(-c4ccc(C(=O)N(C)C)cc4)c3n2)cc2c1CN(CCC(=O)OC(C)(C)C)CC2]

To a solution of tert-butyl 3-(6-(7-(4-(dimethylcarbamoyl)phenyl)-5-tosyl-5H-pyrrolo[2,3-b]pyrazin-2-yl)-8-methyl-3,4-dihydroisoquinolin-2(1H)-yl)propanoate (180 mg, 0.26 mmol) in dioxane (4 mL) and water (2 mL) was added K₂CO₃ (215 mg, 1.56 mmol). The reaction mixture was stirred for 16 h at 100 ° C. then cooled to room temperature. The mixture was diluted with EtOAc (20 mL), washed with H₂O (10 mL), brine (10 mL), dried over Na₂SO₄, filtered and concentrated under vacuum. The residue was purified by prep-TLC with MeOH: DCM=1:25 to give the title compound (110 mg, 79%). LC-MS (M+H)⁺=540.4.

### Step 6: 3-(6-(7-(4-(dimethylcarbamoyl)phenyl)-5H-pyrrolo[2,3-b]pyrazin-2-yl)-8-methyl-3,4-dihydroisoquinolin-2(1H)-yl)propanoic acid trifluoroacetic acid

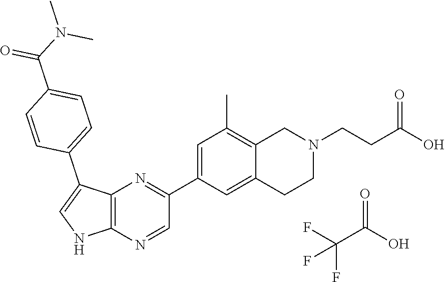

To a solution of tert-butyl 3-(6-(7-(4-(dimethylcarbamoyl)phenyl)-5H-pyrrolo[2,3-b]pyrazin-2-yl)-8-methyl-3,4-dihydroisoquinolin-2(1H)-yl)propanoate (110 mg, 0.2 mmol) in DCM (4 mL) was added TFA (2 mL) dropwise at 0° C. The reaction mixture was stirred for 1 h at room temperature and then concentrated under vacuum to give the title compound (120 mg, 99%). LC-MS (M+H)⁺=484.3.

### Step 7: 4-(2-(2-(3-(4-(4-(2,4-dioxotetrahydropyrimidin-1(2H)-yl)phenyl)piperazin-1-yl)-3-oxopropyl)-8-methyl-1,2,3,4-tetrahydroisoquinolin-6-yl)-5H-pyrrolo[2,3-b]pyrazin-7-yl)-N,N-dimethylbenzamide

To a solution of 3-(6-(7-(4-(dimethylcarbamoyl)phenyl)-5H-pyrrolo[2,3-b]pyrazin-2-yl)-8-methyl-3,4-dihydroisoquinolin-2(1H)-yl)propanoic acid trifluoroacetic acid (100 mg, 0.17 mmol) in DMF (4 mL) was added Et3N (121 mg, 1.2 mmol), EDCI (77 mg, 0.4 mmol), HOBT (54 mg, 0.40 mmol) and 1-(4-(piperazin-1-yl)phenyl)dihydropyrimidine-2,4(1H,3H)-dione hydrochloride (57 mg, 0.20 mmol). The reaction mixture was stirred for overnight at room temperature, diluted with EtOAc (20 mL), washed with brine (2×10 mL), dried over Na₂SO₄, filtered and concentrated under vacuum. The residue was purified by TLC with MeOH: DCM=1:8, the resulting crude product was purified by prep-HPLC to give the title compound (17 mg, 11%) as a white solid. LC-MS (M+H)+=740.4.

¹H NMR (400 MHz, DMSO) δ12.40 (s, 1H), 10.28 (s, 1H), 8.88 (s, 1H), 8.53-8.50 (m, 1H), 8.40-8.36 (m, 2H), 7.84 (s, 1H), 7.79 (s, 1H), 7.54-7.49 (m, 2H), 7.18-7.14 (m, 2H), 6.98-6.94 (m, 2H), 3.71-3.65 (m, 4H), 3.64-3.61 (m, 2H), 3.60 -3.57 (m, 2H), 3.19-3.14 (m, 2H), 3.12-3.08 (m, 2H), 3.01 (s, 6H), 2.97-2.91 (m, 2H), 2.87-2.81 (m, 2H), 2.77-2.70 (m, 4H), 2.69-2.64 (m, 2H), 2.31 (s, 3H).

### Example 6: 4-(5-(2-(3-(4-(4-(2,4-dioxotetrahydropyrimidin-1(2H)-yl)phenyl)piperazin-1-yl)-3-oxopropyl)-8-methyl-1,2,3,4-tetrahydroisoquinolin-6-yl)-1H-pyrrolo[2,3-b]pyridin-3-yl)-N,N-dimethylbenzamide

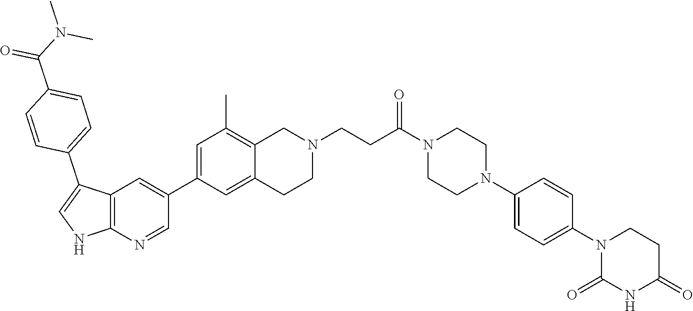 [SMILES: Cc1cc(-c2cnc3[nH]cc(-c4ccc(C(=O)N(C)C)cc4)c3c2)cc2c1CN(CCC(=O)N1CCN(c3ccc(N4CCC(=O)NC4=O)cc3)CC1)CC2]

### Step 1: 4-(5-bromo-1-tosyl-1H-pyrrolo[2,3-b]pyridin-3-yl)-N,N-dimethylbenzamide

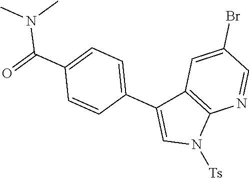

To a solution of 5-bromo-3-iodo-1-tosyl-1H-pyrrolo[2,3-b]pyridine (2.38 g, 5 mmol) in dioxane (30 mL) and water (3 mL) was added (4-(dimethylcarbamoyl)phenyl)boronic acid (965 mg, 5 mmol), K₂CO₃ (1.38 g, 10 mmol) and Pd(dppf)Cl₂·CH₂Cl₂ (409 mg, 0.5 mmol), and the mixture was stirred for 16 h at 90° C. under N₂. The mixture was cooled to room temperature, diluted with EtOAc (50 mL), washed with H₂O (30 mL), brine (30 mL), dried over Na₂SO₄, filtered and concentrated under vacuum. The residue was purified by silica gel chromatograph with PE: EtOAc=1:5 to give the title compound (1.50 g, 60%). LC-MS (M+H)⁺=498.1, 500.1.

### Step 2: tert-butyl 6-(3-(4-(dimethylcarbamoyl)phenyl)-1-tosyl-1H-pyrrolo[2,3-b]pyridin-5-y1)-8-methyl-3,4-dihydroisoquinoline-2(1H)-carboxylate

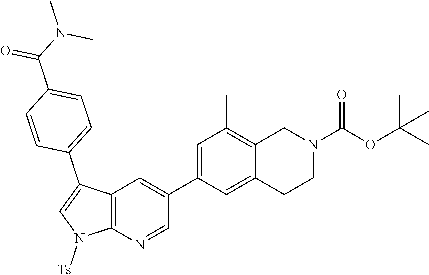 [SMILES: Cc1ccc(S(=O)(=O)n2cc(-c3ccc(C(=O)N(C)C)cc3)c3cc(-c4cc(C)c5c(c4)CCN(C(=O)OC(C)(C)C)C5)cnc32)cc1]

To a solution of 4-(5-bromo-1-tosyl-1H-pyrrolo[2,3-b]pyridin-3-yl)-N,N-dimethylbenzamide (994 mg, 2 mmol) in dioxane (10 mL) and water (1 mL) were added tert-butyl 8-methyl-6-(4,4,5,5-tetramethyl-1,3,2-dioxaborolan-2-yl)-3,4-dihydro-1H-isoquinoline-2-carboxylate (746 mg, 2 mmol), K₂CO₃ (828 mg, 6 mmol) and Pd(dppf)Cl₂·CH₂Cl₂ (164 mg, 0.2 mmol), then stirred for 3 h at 100° C. under N₂. The mixture was cooled to room temperature, diluted with EtOAc (30 mL), washed with H₂O (20 mL), brine (20 mL), dried over Na₂SO₄, filtered and concentrated under vacuum. The residue was purified by silica gel chromatography with EtOAc to give the title compound (1.0 g, 75%). LC-MS (M+H)⁺=665.4.

### Step 3: N,N-dimethyl-4-(5-(8-methyl-1,2,3,4-tetrahydroisoquinolin-6-yl)-1-tosyl-1H-pyrrolo[2,3-b]pyridin-3-yl)benzamide hydrochloride

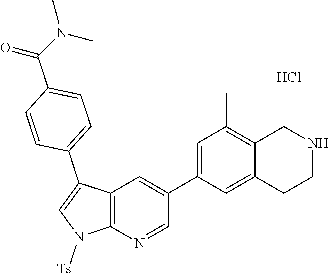 [SMILES: Cc1ccc(S(=O)(=O)n2cc(-c3ccc(C(=O)N(C)C)cc3)c3cc(-c4cc(C)c5c(c4)CCNC5)cnc32)cc1 ; Cl]

To a solution of tert-butyl 6-(3-(4-(dimethylcarbamoyl)phenyl)-1-tosyl-1H-pyrrolo[2,3-b]pyridin-5-yl)-8-methyl-3,4-dihydroisoquinoline-2(1H)-carboxylate (1.0 g, 1.5 mmol) in dioxane (2 mL) was added HCl/dioxane (4 M, 10 mL) dropwise at 0° C. The reaction mixture was stirred for 1 h at room temperature and then concentrated under vacuum to give the title compound (0.90 g, 99%). LC-MS (M+H)⁺=565.3.

### Step 4: tert-butyl 3-(6-(3-(4-(dimethylcarbamoyl)phenyl)-1-tosyl-1H-pyrrolo[2,3-b]pyridin-5-yl)-8-methyl-3,4-dihydroisoquinolin-2(1H)-yl)propanoate

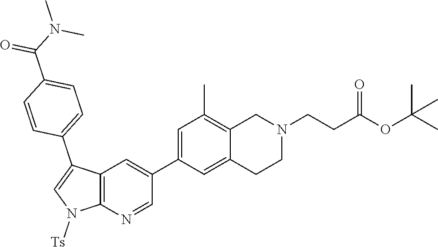 [SMILES: Cc1ccc(S(=O)(=O)n2cc(-c3ccc(C(=O)N(C)C)cc3)c3cc(-c4cc(C)c5c(c4)CCN(CCC(=O)OC(C)(C)C)C5)cnc32)cc1]

To a solution of N,N-dimethyl-4-(5-(8-methyl-1,2,3,4-tetrahydroisoquinolin-6-yl)-1-tosyl-1H-pyrrolo[2,3-b]pyridin-3-yl)benzamide hydrochloride (400 mg, 0.67 mmol) in DMF (5 mL) was added tert-butyl 3-bromopropanoate (890 mg, 4.26 mmol). The reaction mixture was stirred for overnight at 40 ° C., diluted with EtOAc (30 mL), washed with brine (2×15 mL), dried over Na₂SO₄, filtered and concentrated under vacuum. The residue was purified by prep-TLC with MeOH: DCM=1:25 to give the title compound (200 mg, 43%). LC-MS (M+H)⁺=693.4.

### Step 5: tert-butyl 3-(6-(3-(4-(dimethylcarbamoyl)phenyl)-1H-pyrrolo[2,3-b]pyridin-5-yl)-8-methyl-3,4-dihydroisoquinolin-2(1H)-yl)propanoate

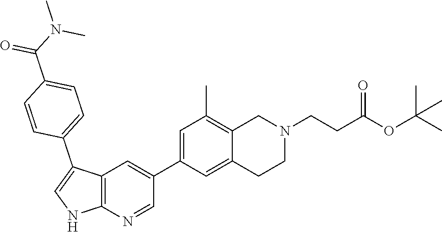 [SMILES: Cc1cc(-c2cnc3[nH]cc(-c4ccc(C(=O)N(C)C)cc4)c3c2)cc2c1CN(CCC(=O)OC(C)(C)C)CC2]

To a solution of tert-butyl 3-(6-(3-(4-(dimethylcarbamoyl)phenyl)-1-tosyl-1H-pyrrolo[2,3-b]pyridin-5-yl)-8-methyl-3,4-dihydroisoquinolin-2(1H)-yl)propanoate (200 mg, 0.29 mmol) in dioxane (4 mL) and water (2 mL) was added K₂CO₃ (239 mg, 1.73 mmol). The reaction mixture was stirred for 16 h at 100° C., diluted with EtOAc (20 mL), washed with H₂O (10 mL), brine (10 mL), dried over Na₂SO₄, filtered and concentrated under vacuum. The residue was purified by prep-TLC with MeOH: DCM=1:25 to give the title compound (120 mg, 77%). LC-MS (M+H)⁺=539.4.

Step 6: 3-(6-(3-(4-(dimethylcarbamoyl)phenyl)-1H-pyrrolo[2,3-b]pyridin-5-yl)-8-methyl-3,4-dihydroisoquinolin-2(1H)-yl)propanoic acid trifluoroacetic acid

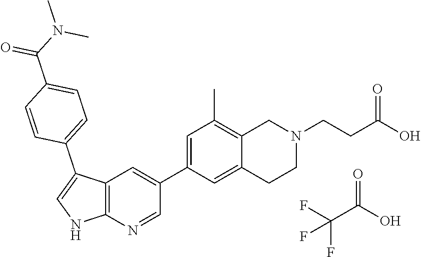

To a solution of tert-butyl 3-(6-(3-(4-(dimethylcarbamoyl)phenyl)-1H-pyrrolo[2,3-b]pyridin-5-yl)-8-methyl-3,4-dihydroisoquinolin-2(1H)-yl)propanoate (120 mg, 0.22 mmol) in DCM (4 mL) was added TFA (2 mL) dropwise at 0° C. The reaction mixture was stirred for 1 h at room temperature and then concentrated under vacuum to give the title compound (132 mg, 100%). LC-MS (M+H)⁺=483.3.

### Step 7: 4-(5-(2-(3-(4-(4-(2,4-dioxotetrahydropyrimidin-1(2H)-yl)phenyl)piperazin-1-yl)-3-oxopropyl)-8-methyl-1,2,3,4-tetrahydroisoquinolin-6-yl)-1H-pyrrolo[2,3-b]pyridin-3-yl)-N,N-dimethylbenzamide

To a solution of 3-(6-(3-(4-(dimethylcarbamoyl)phenyl)-1H-pyrrolo[2,3-b]pyridin-5-yl)-8-methyl-3,4-dihydroisoquinolin-2(1H)-yl)propanoic acid trifluoroacetic acid (90 mg, 0.15 mmol) in DMF (4 mL) was added Et₃N (121 mg, 1.2 mmol), EDCI (73 mg, 0.38 mmol), HOBT (51 mg, 0.38 mmol) and 1-(4-(piperazin-1-yl)phenyl)dihydropyrimidine-2,4(1H,3H)-dione hydrochloride (51 mg, 019 mmol). The reaction mixture was stirred for overnight at room temperature, diluted with EtOAc (20 mL), washed with brine (2×10 mL), dried over Na₂SO₄, filtered and concentrated under vacuum. The residue was purified by prep-TLC with MeOH: DCM=1:8, then purified by prep-HPLC to give the title compound (20 mg, 15%). ¹H NMR (400 MHz, DMSO) δ12.07 (s, 1H), 10.28 (s, 1H), 8.54 (s, 1H), 8.42 (s, 1H), 8.02-7.98 (m, 1H), 7.87-7.82 (m, 2H), 7.52-7.47 (m, 2H), 7.39 (s, 1H), 7.34 (s, 1H), 7.19-7.14 (m, 2H), 6.99-6.93 (m, 2H), 3.71-3.65 (m, 4H), 3.64-3.61 (m, 2H), 3.58-3.53 (m, 2H), 3.19-3.14 (m, 2H), 3.12-3.08 (m, 2H), 3.01 (s, 6H), 2.97-2.91 (m, 2H), 2.87-2.81 (m, 2H), 2.74-2.66 (m, 6H), 2.27 (s, 3H). LC-MS (M+H)⁺=739.4.

### Example 7: (S)-4-(2-(2-(3-(4-(4-(2,4-dioxotetrahydropyrimidin-1(2H)-yl)phenyl)piperazin-1-yl)-3-oxopropyl)-8-methyl-1,2,3,4- tetrahydroisoquinolin-6-yl)-5H-pyrrolo[2,3-b]pyrazin-7-yl)—N-(2-hydroxypropyl)—N-methylbenzamide

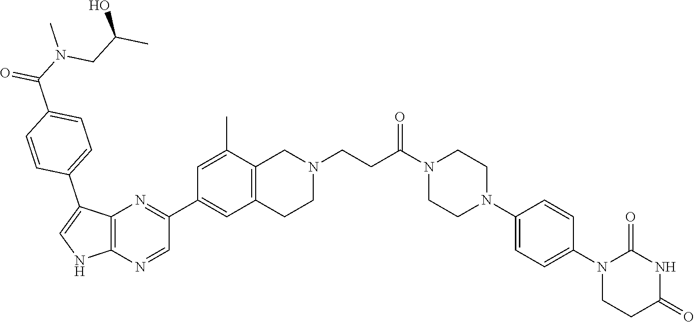 [SMILES: Cc1cc(-c2cnc3[nH]cc(-c4ccc(C(=O)N(C)C[C@H](C)O)cc4)c3n2)cc2c1CN(CCC(=O)N1CCN(c3ccc(N4CCC(=O)NC4=O)cc3)CC1)CC2]

### Step 1: (S)-4-bromo—N-(2-hydroxypropyl)benzamide

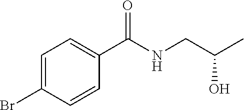

To solution of 4-bromobenzoic acid (11.80 g, 55.8 mmol) in DMF (120 mL) was added DIPEA (10.50 g, 104 mmol), HATU (22.40 g, 58.9 mmol) and (2S)-1-aminopropan-2-ol (4.00 g, 53.3 mmol). The resulting mixture was stirred for 6 h at room temperature. The reaction was quenched by addition of water. The resulting mixture was extracted with EtOAc (600 mL×3). The combined organic layers were washed with brine (100 mL), dried over anhydrous Na₂SO₄. After filtration, the filtrate was concentrated under reduced pressure. The residue was purified by silica gel column chromatography, eluted with PE/EtOAc (30:70) to give the title compound (12.0 g, 88%). LC-MS (M+H)⁺=258.1

### Step 2: (S)-4-bromo—N-(2-((tert-butyldimethylsilyl)oxy)propyl)benzamide

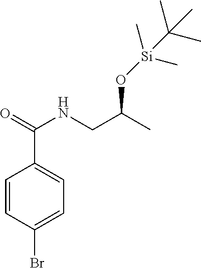 [SMILES: C[C@@H](CNC(=O)c1ccc(Br)cc1)O[Si](C)(C)C(C)(C)C]

(S)-4-bromo—N-(2-hydroxypropyl)benzamide (2.45 g, 9.5 mmol), TBSCl (1.56 g, 10.4 mmol) and Et₃N (2.6 mL, 19.0 mmol) was added to DCM (40 mL) at room temperature. The mixture was stirred overnight. Water (40 mL) was added and the organic layer was separated. The aqueous layer was extracted with EtOAc (30 mL). Combined organic layer was washed with brine (40 mL), dried over Na₂SO₄, filtered and conecentrated to give the title compound (2.9 g, 83%). LC-MS (M+H)⁺=372.2.

### Step 3: (S)-4-bromo—N-(2-((tert-butyldimethylsilyl)oxy)propyl)—N-methylbenzamide

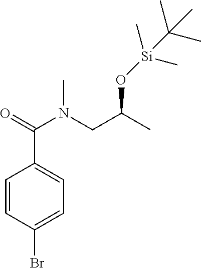 [SMILES: C[C@@H](CN(C)C(=O)c1ccc(Br)cc1)O[Si](C)(C)C(C)(C)C]

To a solution of (S)-4-bromo—N-(2-((tert-butyldimethylsilyl)oxy)propyl)benzamide (2.9 g, 7.8 mmol) in DMF (20 mL) was added NaH (470 mg, 11.7 mmol) at 0° C. and stirred for 1 h, then CH₃I (1.3 g, 9.4 mmol) was added. The reaction mixture was warmed to room temperature and stirred for 3 h. Water (80 mL) was added and the mixture was extracted with EtOAc (50 mL). The organic layer was dried over Na₂SO₄, filtered and concentrated in vacuo to give the title compound (3.0 g, 100%). LC-MS (M+H)⁺=386.1.

### Step 4: (S)—N-(2-((tert-butyldimethylsilyl)oxy)propyl)—N-methyl-4-(4,4,5,5-tetramethyl-1,3,2-dioxaborolan-2-yl)benzamide

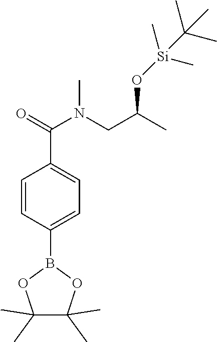 [SMILES: C[C@@H](CN(C)C(=O)c1ccc(B2OC(C)(C)C(C)(C)O2)cc1)O[Si](C)(C)C(C)(C)C]

A mixture of (S)-4-bromo—N-(2-((tert-butyldimethylsilyl)oxy)propyl)—N-methylbenzamide (3.0 g, 7.8 mmol), BPD (2.4 g, 9.3 mmol), Pd(dppf)Cl₂ (400 mg, 0.54 mmol) and AcOK (1.5 g, 15.5 mmol) was in dioxane (40 mL) was heated to reflux under nitrogen for overnight. The mixture was cooled to room temperature, concentrated and the residue was purified by prep-TLC (PE/EtOAc=4:1) to give the title compound (3.6 g, quantitative).

### Step 5: (S)-4-(2-bromo-5H-pyrrolo[2,3-b]pyrazin-7-yl)-N-(2-((tert-butyldimethylsilyl)oxy)propyl)-N-methylbenzamide

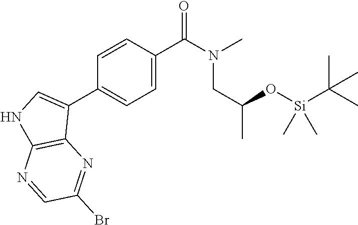 [SMILES: C[C@@H](CN(C)C(=O)c1ccc(-c2c[nH]c3ncc(Br)nc23)cc1)O[Si](C)(C)C(C)(C)C]

A mixture of 2-bromo-7-iodo-5-tosyl-5H-pyrrolo[2,3-b]pyrazine (1.1 g, 2.3 mmol), (S)-N-(2-((tert-butyldimethylsilyl)oxy)propyl)-N-methyl-4-(4,4,5,5-tetramethyl-1,3,2-dioxaborolan-2-yl)benzamide (1.0 g, 2.3 mmol), Pd(PPh3)4 (133 mg, 0.12 mmol) and Na2CO3 (294 mg, 2.8 mmol) in dioxane (40 mL) and water (10 mL) was heated to refluxed overnight under nitrogen. The mixture was cooled to room temperature, concentrated in vacuo and the residue was purified by silica gel chromatography, eluting with PE/EtOAc=2:1 to give the title compound (690 mg, 60%). LC-MS (M+H)⁺=503.1.

### Step 6: tert-butyl (S)-6-(7-(4-((2-((tert-butyldimethylsilyl)oxy)propyl)(methyl)carbamoyl)phenyl)-5H-pyrrolo[2,3-b]pyrazin-2-yl)-8-methyl-3,4- dihydroisoquinoline-2(1H)-carboxylate

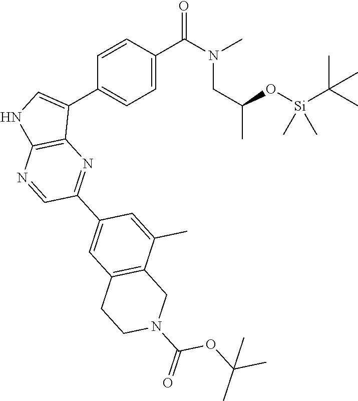 [SMILES: Cc1cc(-c2cnc3[nH]cc(-c4ccc(C(=O)N(C)C[C@H](C)O[Si](C)(C)C(C)(C)C)cc4)c3n2)cc2c1CN(C(=O)OC(C)(C)C)CC2]

The title compound (560 mg, 61%) was prepared in a manner similar to that in Example 1 step 12 from tert-butyl 8-methyl-6-(4,4,5,5-tetramethyl-1,3,2-dioxaborolan-2-yl)-3,4-dihydro-1H-isoquinoline-2-carboxylate and (S)-4-(2-bromo-5H-pyrrolo[2,3-b]pyrazin-7-yl)-N-(2-((tert-butyldimethylsilyl)oxy)propyl)-N-methylbenzamide. LC-MS (M+H)⁺=670.3.

### Step 7: (S)—N-(2-hydroxypropyl)-N-methyl-4-(2-(8-methyl-1,2,3,4-tetrahydroisoquinolin-6-yl)-5H-pyrrolo[2,3-b]pyrazin-7-yl)benzamide hydrochloride

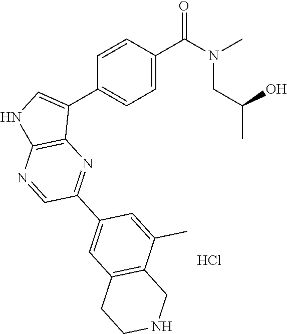 [SMILES: Cc1cc(-c2cnc3[nH]cc(-c4ccc(C(=O)N(C)C[C@H](C)O)cc4)c3n2)cc2c1CNCC2 ; Cl]

The title compound (460 mg, quantitative) was prepared in a manner similar to that in Example 1 step 14 from tert-butyl (S)-6-(7-(4-((2-((tert-butyldimethylsilyl)oxy)propyl)(methyl)carbamoyl)phenyl)-5H-pyrrolo[2,3-b]pyrazin-2-yl)-8-methyl-3,4-dihydroisoquinoline-2(1H)-carboxylate. LC-MS (M+H)⁺=456.2.

### Step 8: tert-butyl (S)-3-(6-(7-(4-((2-hydroxypropyl)(methyl)carbamoyl)phenyl)-5H-pyrrolo[2,3-b]pyrazin-2-yl)-8-methyl-3,4-dihydroisoquinolin-2(1H)-yl)propanoate

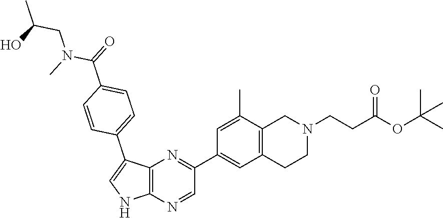 [SMILES: Cc1cc(-c2cnc3[nH]cc(-c4ccc(C(=O)N(C)C[C@H](C)O)cc4)c3n2)cc2c1CN(CCC(=O)OC(C)(C)C)CC2]

A mixture of (S)-N-(2-hydroxypropyl)-N-methyl-4-(2-(8-methyl-1,2,3,4-tetrahydroisoquinolin-6-yl)-5H-pyrrolo[2,3-b]pyrazin-7-yl)benzamide hydrochloride (460 mg, 0.93 mmol), tert-butyl 3-bromopropanoate (176 mg, 0.84 mmol) and K₂CO₃ (322 mg, 23. 4 mmol) in DMF (5 mL) was stirred overnight at room temperature. Water (40 mL) was added and the mixture was extracted with EtOAc (20 mL). The organic layer was dried over Na₂SO₄, filtered and concentrated in vacuo. The residue was purified by prep-TLC (DCM/MeOH=8:1) to give the title compound (200 mg, 37%). LC-MS (M+H)⁺=584.3.

### Step 9: (S)-3-(6-(7-(4-((2-hydroxypropyl)(methyl)carbamoyl)phenyl)-5H-pyrrolo[2,3-b]pyrazin-2-yl)-8-methyl-3,4-dihydroisoquinolin-2(1H)-yl)propanoic acid hydrochloride

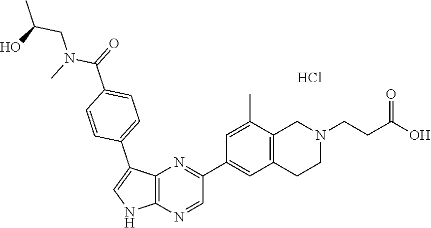 [SMILES: Cc1cc(-c2cnc3[nH]cc(-c4ccc(C(=O)N(C)C[C@H](C)O)cc4)c3n2)cc2c1CN(CCC(=O)O)CC2 ; Cl]

The title compound (460 mg, quantitative) was prepared in a manner similar to that in Example 2 step 2 from tert-butyl (S)-3-(6-(7-(4-((2-hydroxypropyl)(methyl)carbamoyl)phenyl)-5H-pyrrolo[2,3-b]pyrazin-2-yl)-8-methyl-3,4-dihydroisoquinolin-2(1H)-yl)propanoate. LC-MS (M+H)⁺=528.2.

### Step 10: (S)-4-(2-(2-(3-(4-(4-(2,4-dioxotetrahydropyrimidin-1(2H)-yl)phenyl)piperazin-1-y1)-3-oxopropyl)-8-methyl-1,2,3,4-tetrahydroisoquinolin-6-yl)-5H-pyrrolo[2,3-b]pyrazin-7-yl)-N-(2-hydroxypropyl)-N-methylbenzamide

Example 7 (20 mg, 21%) was prepared in a manner similar to that in Example 1 step 18 from 3-methyl-1-(4-(piperazin-1-yl)phenyl)dihydropyrimidine-2,4(1H,3H)-dione hydrochloride and (S)-3-(6-(7-(4-((2-hydroxypropyl)(methyl)carbamoyl)phenyl)-5H-pyrrolo[2,3-b]pyrazin-2-yl)-8-methyl-3,4-dihydroisoquinolin-2(1H)-yl)propanoic acid hydrochloride. ¹H NMR (400 MHz, DMSO) δ12.38 (s, 1H), 10.24 (s, 1H), 8.87 (s, 1H), 8.48 (s, 1H), 8.33 (s, 2H), 7.85 (s, 2H), 7.47 (d, J=7.7 Hz, 2H), 7.14 (d, J=8.8 Hz, 2H), 6.94 (d, J=8.5 Hz, 2H), 4.83-4.79 (m, 1H), 4.00-3.90 (m, 1H), 3.85-3.79 (m, 1H), 3.69-3.57 (m, 7H), 3.47 (s, 1H), 3.27 (s, 3H), 3.20-3.12 (m, 3H), 3.12-3.05 (m, 4H), 3.05-2.97 (m, 4H), 2.63 (t, J=6.6 Hz, 3H), 2.30 (s, 3H), 1.14-1.08 (m, 2H), 0.90-0.85 (m, 2H). LC-MS (M+H)⁺=784.3.

### Example 8: 4-(2-(2-(2-(2-(1-(4-(2,6-dioxopiperidin-3-yl)phenyl)piperidin-4-yl)acetamido)ethyl)-8-methyl-1,2,3,4-tetrahydroisoquinolin-6-yl)-5H-pyrrolo[2,3-b]pyrazin-7-yl)-N,N,2-trimethylbenzamide

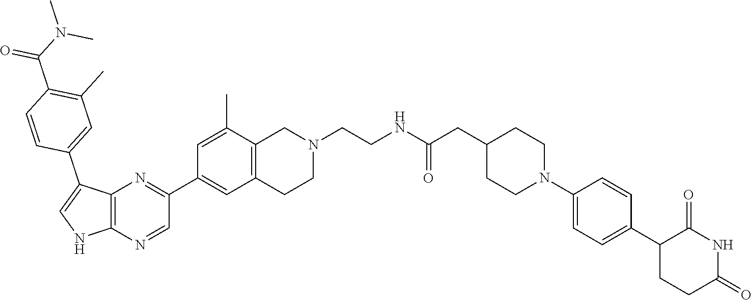 [SMILES: Cc1cc(-c2c[nH]c3ncc(-c4cc(C)c5c(c4)CCN(CCNC(=O)CC4CCN(c6ccc(C7CCC(=O)NC7=O)cc6)CC4)C5)nc23)ccc1C(=O)N(C)C]

Example 8 (25 mg, 17%) was prepared in a manner similar to that in Example 1 step 18 from 2-(1-(4-(2,6-dioxopiperidin-3-yl)phenyl)piperidin-4-yl)acetic acid and 4-(2-(2-(2-aminoethyl)-8-methyl-1,2,3,4-tetrahydroisoquinolin-6-yl)-5H-pyrrolo[2,3-b]pyrazin-7-yl)-N,N,2-trimethylbenzamide dihydrochloride. ¹H NMR (400 MHz, DMSO) δ12.31 (s, 1H), 10.68 (s, 1H), 8.86 (s, 1H), 8.42 (s, 1H), 8.20 (s, 1H), 8.13 (d, J=8.4 Hz, 1H), 7.83 (s, 3H), 7.17 (d, J=7.9 Hz, 1H), 6.70 (s, 4H), 3.57-3.48 (m, 3H), 3.26 (s, 3H), 2.96 (s, 3H), 2.95-2.83 (m, 2H), 2.76 (s, 3H), 2.74-2.65 (m, 1H), 2.65-2.55 (m, 1H), 2.55-2.48 (m, 2H), 2.40-2.31 (m, 3H), 2.25 (s, 3H), 2.25-2.20 (m, 5H), 2.03-1.90 (m, 4H), 1.72-1.60 (m, 2H), 1.70-1.62 (m,3H), 1.20-1.16 (m, 3H). LC-MS (M+H)⁺=781.9.

### Example 9: (S)-4-(5-(2-(3-(4-(4-(2,4-dioxotetrahydropyrimidin-1(2H)-yl)phenyl)piperazin-1-yl)-3-oxopropyl)-8-methyl-1,2,3,4-tetrahydroisoquinolin-6-yl)-1H-pyrrolo[2,3-b]pyridin-3-yl)-N-(2-hydroxypropyl)-N-methylbenzamide

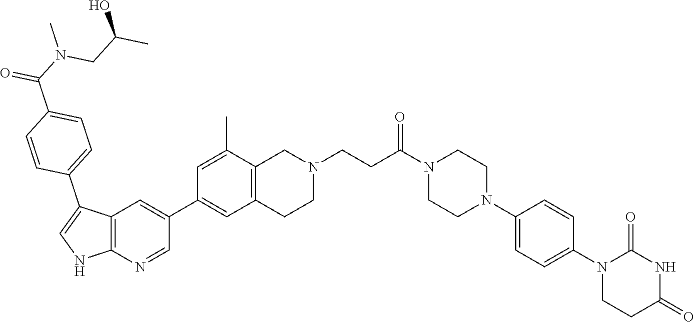 [SMILES: Cc1cc(-c2cnc3[nH]cc(-c4ccc(C(=O)N(C)C[C@H](C)O)cc4)c3c2)cc2c1CN(CCC(=O)N1CCN(c3ccc(N4CCC(=O)NC4=O)cc3)CC1)CC2]

### Step 1: 5-bromo-3-iodo-1H-pyrrolo[2,3-b]pyridine

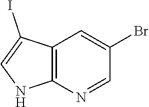 [SMILES: Brc1cnc2[nH]cc(I)c2c1]

To a solution of 5-bromo-1H-pyrrolo[2,3-b]pyridine (5.0 g, 25.4 mmol) in THF (80 mL) was added NIS (6.85 g, 30.4 mmol, 1.2 eq) at room temperature. After 2 h, the reaction mixture was concentrated under reduced pressure. The crude was purified by silica gel chromatography with PE/EtOAc (15/1) to give the title compound (8.0 g, 97%). LCMS (M+H)⁺=322.6.

### Step 2: tert-butyl 5-bromo-3-iodo-1H-pyrrolo[2,3-b]pyridine-1-carboxylate

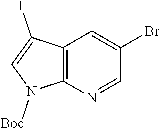 [SMILES: CC(C)(C)OC(=O)n1cc(I)c2cc(Br)cnc21]

A solution of 5-bromo-3-iodo-1H-pyrrolo[2,3-b]pyridine (8.0 g, 24.7 mmol), Boc₂O (7.5 g, 34.6 mmol) and Et₃N (7.4 g, 74 mmol) in THF (80 mL) was stirred at room temperature for overnight. The reaction mixture was concentrated under reduced pressure. The crude was purified by silica gel chromatography with PE/EtOAc (20/1) to give the title compound (10 g, 95%). LCMS (M+H)⁺=422.6.

### Step 3: (S)-4-(5-bromo-1H-pyrrolo[2,3-b]pyridin-3-yl)-N-(2-((tert-butyldimethylsilyl)oxy)propyl)-N-methylbenzamide

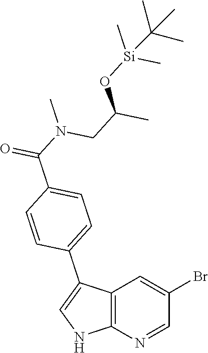 [SMILES: C[C@@H](CN(C)C(=O)c1ccc(-c2c[nH]c3ncc(Br)cc23)cc1)O[Si](C)(C)C(C)(C)C]

A mixture of tert-butyl 5-bromo-3-iodo-1H-pyrrolo[2,3-b]pyridine-1-carboxylate (2.0 g, 4.7 mmol), (S)-N-(2-((tert-butyldimethylsilyl)oxy)propyl)-N-methyl-4-(4,4,5,5-tetramethyl-1,3,2-dioxaborolan-2-yl)benzamide (1.4 g, 3.3 mmol), Pd(dppf)Cl₂ (196 mg, 0.27 mmol) and K₂CO₃ (630 mg, 4.6 mmol) in dioxane (35 mL) and water (10 mL) was heated to reflux overnight. The mixture was cooled to room temperature, then partitioned between EtOAc (30 mL) and water (30 mL). The organic layer was separated, dried over Na₂SO₄, filtered and concentrated in vacuo. The crude was purified by prep-TLC (DCM/MeOH=10:1) to give the title compound (740 mg, 46%). LC-MS (M+H)⁺=502.1.

### Step 4: tert-butyl (S)-6-(3-(4-((2-((tert-butyldimethylsilyl)oxy)propyl)(methyl)carbamoyl)phenyl)-1H-pyrrolo[2,3-b]pyridin-5-yl)-8-methyl-3,4- dihydroisoquinoline-2(1H)-carboxylate

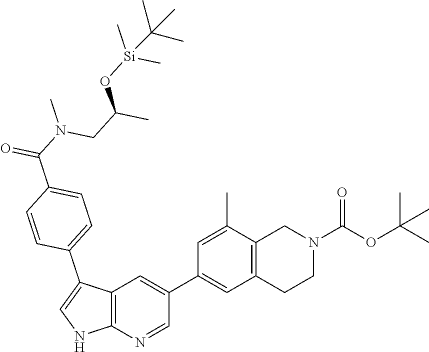 [SMILES: Cc1cc(-c2cnc3[nH]cc(-c4ccc(C(=O)N(C)C[C@H](C)O[Si](C)(C)C(C)(C)C)cc4)c3c2)cc2c1CN(C(=O)OC(C)(C)C)CC2]

The title compound was prepared in a manner similar to that in Example 1 step 12 from (S)-4-(5-bromo-1H-pyrrolo[2,3-b]pyridin-3-yl)-N-(2-((tert-butyldimethylsilyl)oxy)propyl)—N-methylbenzamide and tert-butyl 8-methyl-6-(4,4,5,5-tetramethyl-1,3,2-dioxaborolan-2-yl)-3,4-dihydroisoquinoline-2(1H)-carboxylate (220 mg, 61%). LC-MS (M+H)⁺=669.3.

### Step 5: (S)—N-(2-hydroxypropyl)—N-methyl-4-(5-(8-methyl-1,2,3,4-tetrahydroisoquinolin-6-yl)-1H-pyrrolo[2,3-b]pyridin-3-yl)benzamide hydrochloride

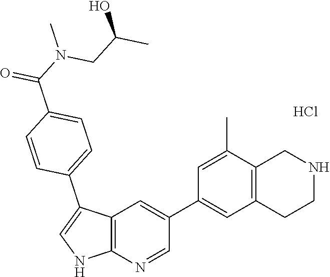 [SMILES: Cc1cc(-c2cnc3[nH]cc(-c4ccc(C(=O)N(C)C[C@H](C)O)cc4)c3c2)cc2c1CNCC2 ; Cl]

The title componud was prepared in a manner similar to that in Example 6 step 3 from tert-butyl (S)-6-(3-(4-((2-((tert-butyldimethylsilyl)oxy)propyl)(methyl)carbamoyl)phenyl)-1H-pyrrolo[2,3-b]pyridin-5-yl)-8-methyl-3,4-dihydroisoquinoline-2(1H)-carboxylate (150 mg, 86%). LC-MS (M+H)⁺=455.2.

### Step 6: tert-butyl (S)-3-(6-(3-(4-((2-hydroxypropyl)(methyl)carbamoyl)phenyl)-1H-pyrrolo[2,3-b]pyridin-5-yl)-8-methyl-3,4-dihydroisoquinolin-2(1H)-yl)propanoate

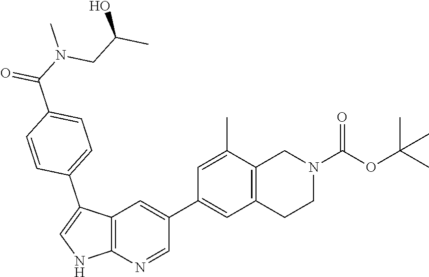 [SMILES: Cc1cc(-c2cnc3[nH]cc(-c4ccc(C(=O)N(C)C[C@H](C)O)cc4)c3c2)cc2c1CN(C(=O)OC(C)(C)C)CC2]

The title compound was prepared in a manner similar to that in Example 7 step 8 from (S)-N-(2-hydroxypropyl)—N-methyl-4-(5-(8-methyl-1,2,3,4-tetrahydroisoquinolin-6-yl)-1H-pyrrolo[2,3-b]pyridin-3-yl)benzamide hydrochloride and tert-butyl 3-bromopropanoate (50 mg, 31%). LC-MS (M+H)⁺=583.3.

### Step 7: (S)-3-(6-(3-(4-((2-hydroxypropyl)(methyl)carbamoyl)phenyl)-1H-pyrrolo[2,3-b]pyridin-5-yl)-8-methyl-3,4-dihydroisoquinolin-2(1H)-yl)propanoic acid hydrochloride

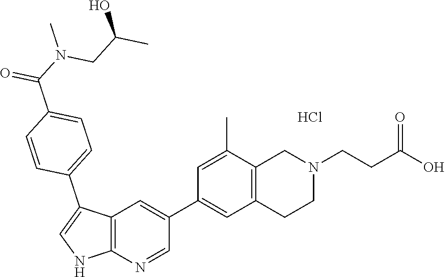 [SMILES: Cc1cc(-c2cnc3[nH]cc(-c4ccc(C(=O)N(C)C[C@H](C)O)cc4)c3c2)cc2c1CN(CCC(=O)O)CC2 ; Cl]

The title compound was prepared in a manner similar to that in Example 2 step 2 from tert-butyl (S)-3-(6-(3-(4-((2-hydroxypropyl)(methyl)carbamoyl)phenyl)-1H-pyrrolo[2,3-b]pyridin-5-yl)-8-methyl-3,4-dihydroisoquinolin-2(1H)-yl)propanoate (45 mg, 94%). LC-MS (M+H)⁺=527.2.

### Step 8: (S)-4-(5-(2-(3-(4-(4-(2,4-dioxotetrahydropyrimidin-1(2H)-yl)phenyl)piperazin-1-yl)-3-oxopropyl)-8-methyl-1,2,3,4-tetrahydroisoquinolin-6-yl)-1H-pyrrolo[2,3-b]pyridin-3-yl)—N-(2-hydroxypropyl)—N-methylbenzamide

Example 9 was prepared in a manner similar to that in Example 3 step 6 from (S)-3-(6-(3-(4-((2-hydroxypropyl)(methyl)carbamoyl)phenyl)-1H-pyrrolo[2,3-b]pyridin-5-yl)-8-methyl-3,4-dihydroisoquinolin-2(1H)-yl)propanoic acid hydrochloride and 1-(4-(piperazin-1-yl)phenyl)dihydropyrimidine-2,4(1H,3H)-dione hydrochloride (15 mg, 7%). ¹H NMR (400 MHZ, DMSO) 8 12.06 (s, 1H), 10.27 (s, 1H), 8.54 (s, 1H), 8.42 (s, 1H), 7.99 (d, J=2.5 Hz, 1H), 7.84 (d, J =7.9 Hz, 2H), 7.49 (d, J=8.0 Hz, 2H), 7.39 (s, 1H), 7.34 (s, 1H), 7.16 (d, J=8.8 Hz, 2H), 6.96 (d, J=8.9 Hz, 2H), 4.88-4.83 (m, 1H), 3.99-3.88 (m, 1H), 3.70-3.67 (m, 6H), 3.56 (s, 2H), 3.16 (s, 2H), 3.10 (s, 2H), 3.06-2.97 (m, 4H), 2.90 (s, 2H), 2.83 (t, J=7.0 Hz, 2H), 2.72-2.67 (m, 6H), 2.26 (s, 3H), 1.11-0.92 (m, 3H). LC-MS (M+H) +=783.3.

### BIOLOGICAL ACTIVITY

### HPK Kinase Activity Assay at 1 mM ATP

Compounds disclosed herein were tested for inhibition of HPK1 kinase (aa1-346, Life Technologies) activity in assays based on the time-resolved fluorescence-resonance energy transfer (TR-FRET) methodology. The assays were carried out in 384-well low volume black plates in a reaction mixture containing HPK1 kinase (40 nM), 1 mM ATP, 0.5 μM STK1 substrate and 0-10 μM compound in buffer containing 50 mM HEPES, 0.01% BSA, 0.1 mM Orthovanadate, 10 mM MgCl₂, 1 mM DTT, pH=7.0, 0.005% Tween-20. The kinase was incubated with the compounds disclosed herein or DMSO for 60 minutes at room temperature and the reaction was initiated by the addition of ATP and STK1 substrate. After reaction at room temperature for 120 minutes, an equal volume of stop/detection solution was added according to the manufacture's instruction (CisBio). The stop/detection solution contained STK Antibody—Cryptate and XL665-conjugated streptavidin in Detection Buffer. The TR-FRET signals (ratio of fluorescence emission at 665 nm over emission at 620 nm with excitation at 337 nm wavelength) were recorded on a PHERAstar FS plate reader (BMG Labtech). Phosphorylation of STK1 substrate led to the binding of STK Antibody—Cryptate to the biotinylated STK1 substrate, which places fluorescent donor (Eu³⁺ crypate) in close proximity to the accepter (Streptavidin-XL665), thus resulting in a high degree of fluorescence resonance energy transfer. The inhibition of HPK1 in presence of increasing concentrations of compounds was calculated based on the ratio of fluorescence at 665 nm to that at 620 nm. IC₅₀ determination was performed by fitting the curve of percent inhibition versus the log of the inhibitor concentration using Dotmatics. The compounds disclosed herein showed the enzymatic activity values as in Table 1.

TABLE 1
Enzymatic activity IC₅₀(nM) for the compounds disclosed herein

| Comp | Enzymatic activity | Comp | Enzymatic activity |
|---|---|---|---|
| No. | IC₅₀ (nM) | No. | IC₅₀ (nM) |
| 1 | 121 | 2 | 253 |
| 3 | 61 | 4 | 140 |
| 5 | 24 | 6 | 31 |
| 7 | 20 | 8 | 297 |
| 9 | 16 | | |

### Cellular pSLP76(S376) HTRF Assay

Jurkat cell line was used in this study. Cells were maintained in RPMI 1640 supplemented with heat-inactivated 10% fetal bovine serum (Thermo Fisher), 50 units/mL penicillin and streptomycin (Thermo Fisher) and kept at 37° C. in a humidified atmosphere of 5% CO₂ in air. Cells were reinstated from frozen stocks that were laid down within 30 passages from the original cells purchased. After cell starvation in the assay buffer (RPMI 1640 supplemented with heat-inactivated 0.1% fetal bovine serum) for 18 hours, cells were seeded into a round bottom 96-well plate at 150,000 cells per well density. Cells were treated with a 9-point dilution series of test compounds. The final compound concentration is from 0 to 2 μM. After 2 h compound treatment, cells were stimulated with 0.05 μg/mL anti-human CD3 (OKT3, Bioxcell) for 30 min at 37° C. Then the cells were lysed, and the pSLP76 (S376) level in the cell lysates was detected by HTRF kit (Cisbio). A total of 16 μL of cell lysate from each well of a 96-well plate was transferred to a 384-well small volume white assay plate. Lysate from each well was incubated with 2 μL of Eu³⁺-cryptate (donor) labeled anti-phospho-SLP76 and 2 μL of D2 (acceptor) labeled anti-phospho-SLP76 antibodies (Cisbio) overnight in dark at room temperature. FRET signals (655 nm) were measured using a PHERAstar FSX reader (BMG Labtech). IC₅₀ determination was performed by fitting the curve of percent inhibition versus the log of the inhibitor concentration using Dotmatics. The compounds disclosed herein showed the cellular activity values as in Table 2.

TABLE 2
Cellular activity IC₅₀ (nM) for the compounds disclosed herein

| Comp | Cellular activity | Comp | Cellular activity |
|---|---|---|---|
| No. | IC₅₀ (nM) | No. | IC₅₀ (nM) |
| 1 | 879 | 2 | 515 |
| 3 | 193 | 4 | 459 |
| 5 | 116 | 6 | 88 |
| 7 | 307 | 8 | 496 |
| 9 | 181 | | |

### HPK1 Protein Degradation Assay in Human PBMC (Western Blot)

Human PBMCs (AllCells) was used in this study. Cells were thawed one day in advance and maintained in RPMI-1640 supplemented 10% fetal bovine serum (Thermo Fisher), 50 units/mL penicillin and streptomycin (Thermo Fisher) and the mixture was kept at 37° C. in a humidified atmosphere air containing 5% CO₂. 3×10⁶ PBMCs per well in 1 mL culture medium were seeded in the 12-well plate, and the cells were treated with an appropriate dilution series of compounds with a final concentration up to 10 mM. After 24 h of compound incubation, the medium was aspirated, the cells were washed with PBS, and then 30 μL of 1X protein lysis buffer (Cell Signaling Technology) containing protease inhibitors (Merck) and phosphatase inhibitors (Sigma) was added. The cells were lysed. After centrifugation, the supernatants were quantified by BCA protein assay kit (Thermo Fisher). 4X loading Buffer (Thermo Fisher) was added to equal amounts of total protein from each sample and heated at 95° C. for 5 minutes. 30-50 μg of cell lysate was loaded onto a 4%-12% NuPAGE Bis-Tris Gel (Thermo Fisher) and electro-transferred to NC membranes (Thermo Fisher). The membranes were blocked for least 1 hour with blocking reagent (LI-COR), and then incubated overnight with anti-HPK1 (Cell Signaling Technology, 4472S) antibodies and as loading control anti-ß-actin (Cell Signaling Technology, 3700S) or anti-GAPDH (Cell Signaling Technology, 97166S) at 4° C. with gentle shaking. The membranes were washed three times with TBST, and incubated at least 1 hour at room temperature with anti-mouse or anti-rabbit secondary fluorescent antibody (Thermo Fisher, A32729; LI—COR, 926-32213). Then the membranes were washed three times in TBST, and one time in water. Immunoreactive bands were visualized by Odyssey CLx and analyzed by GraphPad Prism 8. The compounds disclosed herein showed the Dₘₐₓ, DC₅₀ and DC₉₀ values as in Table 3.

TABLE 3
Protein degradation Dₘₐₓ, DC₅₀ and DC₉₀
(nM) for the compounds disclosed herein

| Comp | Dₘₐₓ | DC₅₀ | DC₉₀ | Comp | Dₘₐₓ | DC₅₀ | DC₉₀ |
|---|---|---|---|---|---|---|---|
| No. | (%) | (nM) | (nM) | No. | (%) | (nM) | (nM) |
| 1 | 47 | 183 | 3805 | 2 | 91 | 87 | 294 |
| 3 | 97 | 23 | 215 | 4 | 95 | 59 | 817 |

### HPK1 Protein Degradation Assay in Human PBMC (FACS)

Human PBMCs (AllCells) was used in this study. Cells were thawed one day in advance and maintained in RPMI-1640 supplemented 10% fetal bovine serum (Thermo Fisher), 50 units/mL penicillin and streptomycin (Thermo Fisher) and the mixture was kept at 37° C. in a humidified atmosphere air containing 5% CO₂. 3×10⁶ PBMCs per well in 95 μL culture medium were seed in the 96-well plate, and the cells were treated with an appropriate dilution series of compounds. After 24 h compound treatment, 100 μL 1xLyse/Fix buffer (BD, diluted with water, warm up in 37° C.) was added to cells and the cells were incubated at 37° C. for 10 minutes. After fixed, the cells were transferred to V-bottom plates (Corning), and washed with FACS buffer (cold 2% FBS DPBS). The cells were permeabilized by adding 100 μL pre-chilled BD Phosflow Perm Buffer III (BD) for 30 min on ice. After washed with FACS buffer twice, the cells were incubated with 50 μL antibody cocktail (HPK1(CST)(1:50), anti-human CD4 BV421 (BD)(1:25), anti-human CD8 APC(BD) (1:25)) at room temperature for 1 h. After washed with FACS buffer twice, the cells were further incubated with 50 μL secondary antibody cocktail (Biolegend) (1:50) at room temperature for 1 h. After washed with FACS buffer twice, the signals were detected by BD FACS Celesta. The data were analyzed by Excel and GraphPad Prism 8. The compounds disclosed herein showed the Dₘₐₓ, DC₅₀ and DC₉₀ values as in Table 4.

TABLE 4
Protein degradation Dₘₐₓ, DC₅₀ and DC₉₀ (nM)
for the compounds disclosed herein

| | CD4+ T Cells | | | CD8+ T Cells | | |
|---|---|---|---|---|---|---|
| Comp | Dₘₐₓ | DC₅₀ | DC₉₀ | Dₘₐₓ | DC₅₀ | DC₉₀ |
| No. | (%) | (nM) | (nM) | (%) | (nM) | (nM) |
| 5 | 95 | 9 | 60 | 96 | 17 | 128 |
| 6 | 96 | 11 | 52 | 96 | 16 | 104 |
| 7 | 95 | 25 | 118 | 95 | 45 | 388 |
| 9 | 93 | 19 | 77 | 95 | 71 | 495 |

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

It is to be understood that, if any prior art publication is referred to herein; such reference does not constitute an admission that the publication forms a part of the common general knowledge in the art in any country.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and Examples should not be construed as limiting the scope of the invention.

What is claimed is:

1. A compound of Formula (I): 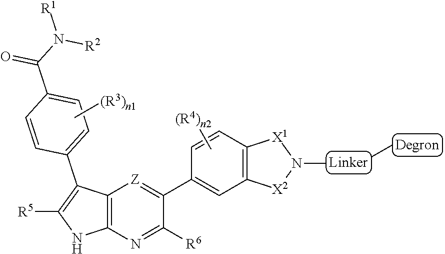
or a pharmaceutically acceptable salt thereof, or a stereoisomer thereof,
wherein
Z is selected from N or CR^{za};
R^{za} is selected from hydrogen, —C₁₋₈alkyl, cycloalkyl;
R¹ and R² are each independently hydrogen, deuterium, —C₁₋₈alkyl, —C₂₋₈alkenyl, —C₂₋₈alkynyl, cycloalkyl, heterocyclyl, aryl, or heteroaryl, each of said —C₁₋₈alkyl, —C₂₋₈alkenyl, —C₂₋₈alkynyl, cycloalkyl, heterocyclyl, aryl, or heteroaryl is optionally substituted with at least one substituents R^{1a}; or
R¹ and R² together with the atom to which they are attached, form a 3- to 12-membered ring, said ring comprising 0, 1 or 2 additional heteroatoms independently selected from nitrogen, oxygen or optionally oxidized sulfur as ring member (s), said ring is optionally substituted with at least one substituents R^{1b};
R^{1a} and R^{1b} are each independently —D (deutrium), halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{1c}, —SO₂R^{1c}, —SO₂NR^{1c}R^{1d}, —COR^{1c}, —CO₂R^{1c}, —CONR^{1c}R^{1d}, —C(═NR^{1c})NR^{1d}R^{1e}, —NR^{1c}R^{1d}, —NR^{1c}COR^{1d}, —NR^{1c}CONR^{1d}R^{1e}, —NR^{1c}CO₂R^{1c}, —NR^{1c}SONR^{1d}R^{1e}, —NR^{1c}SO₂NR^{1d}R^{1e}, or —NR^{1c}SO₂R^{1d}, each of said —C₁₋₈alkyl, —C₂₋₈alkenyl, —C₂₋₈alkynyl, cycloalkyl, heterocyclyl, aryl, or heteroaryl is optionally substituted with at least one substituents selected from halogen, —C₁₋₈alkyl, —OR^{1f}, —NR^{1f}R^{1g}, cycloalkyl, heterocyclyl, aryl, or heteroaryl;
R^{1c}, R^{1d}, R^{1e}, R^{1f}, and R^{1g} are each independently hydrogen, —C₁₋₈alkyl, C₁₋₈alkoxy—C₁₋₈alkyl-, —C₂₋₈alkenyl, —C₂₋₈alkynyl, cycloalkyl, heterocyclyl, aryl, or heteroaryl;
n1 is 0, 1, 2, 3 or 4;
n2 is 0, 1, 2 or 3;
R³, at each of its occurrence, are independently halogen, —C₁₋₈alkyl, —C₂₋₈alkenyl, —C₂₋₈alkynyl, cycloalkyl, heterocyclyl, —C₁₋₈alkyl-heterocyclyl, —C₁₋₈alkyl-cycloalkyl, aryl, heteroaryl, oxo, —CN, —NO₂, —OR^{3a}, —SO₂R^{3a}, —SO₂NR^{3a}R^{3b}, —COR^{3a}, —CO₂R^{3a}, —CONR^{3a}R^{3b}, —C(═NR^{3a})NR^{3b}R^{3c}, —NR^{3a}R^{3b}, —NR^{3a}COR^{3b}, —NR^{3a}CONR^{3b}R^{3c}, —NR^{3a}CO₂R^{3b}, —NR^{3a}SONR^{3b}R^{3c}, —NR^{3a}SO₂NR^{3b}R^{3c}, or —NR^{3a}SO₂R^{3b}, each of said —C₁₋₈alkyl, —C₂₋₈alkenyl, —C₂₋₈alkynyl, cycloalkyl, heterocyclyl, —C₁₋₈alkyl-heterocyclyl, aryl, or heteroaryl is optionally substituted with at least one substituents R^{3d}; or
or when n1=2, two R³, when on adjacent carbon atoms of the phenyl ring, together with the two intervening carbon atoms to which they are attached, form a 5- to 8-membered ring comprising 0, 1 or 2 heteroatoms independently selected from nitrogen, oxygen or optionally oxidized sulfur as ring member (s);
R^{3a}, R^{3b}, and R^{3c} are each independently hydrogen, —C₁₋₈alkyl, —C₂₋₈alkenyl, —C₂₋₈alkynyl, cycloalkyl, heterocyclyl, aryl, or heteroaryl, each of said —C₁₋₈alkyl, —C₂₋₈alkenyl, —C₂₋₈alkynyl, cycloalkyl, heterocyclyl, aryl, or heteroaryl is optionally substituted with at least one substituents R^{3e}; or (R^{3a} and R^{3b}), (R^{3b} and R^{3c}), or (R^{3c} and R^{3a}), together with the atom (s) to which they are attached, form a 3- to 12-membered ring, said ring comprising 0, 1 or 2 additional heteroatoms independently selected from nitrogen, oxygen or optionally oxidized sulfur as ring member (s), said ring is optionally substituted with at least one substituents R^{3e};
R^{3d} and R^{3e} are each independently halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{3f}, —SO₂R^{3f}, —SO₂NR^{3f}R^{3g}, —COR^{3f}, —CO₂R^{3f}, —CONR^{3f}R^{3g}, —C(═NR^{3f})NR^{3g}R^{3h}, —NR^{3f}R^{3g}, —NR^{3f}COR^{3g}, —NR^{3f}CONR^{3g}R^{3h}, —NR^{3a}CO₂R^{3f}, —NR^{3f}SONR^{3f}R^{3g}, —NR^{3f}SO₂NR^{3g}R^{3h}, or —NR^{3f}SO₂R^{3g}, each of said —C₁₋₈alkyl, —C₂₋₈alkenyl, —C₂₋₈alkynyl, cycloalkyl, heterocyclyl, aryl, or heteroaryl is optionally substituted with at least one substituents selected from halogen, —C₁₋₈alkyl, —OR³ⁱ, —NR³ⁱR^{3j}, cycloalkyl, heterocyclyl, aryl, or heteroaryl;
R^{3f}, R^{3g}, R^{3h}, R³ⁱ, and R^{3j} are each independently hydrogen, —C₁₋₈alkyl, C₁₋₈alkoxy—C₁₋₈alkyl-, —C₂₋₈alkenyl, —C₂₋₈alkynyl, cycloalkyl, heterocyclyl, aryl, or heteroaryl;
R⁴, R⁵ and R⁶ are each independently hydrogen, halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{4a}, —SO₂R^{4a}, —COR^{4a}, —CO₂R^{4a}, —CONR^{4a}R^{4b}, —C(═NR^{4a})NR^{4b}R^{4c}, —NR^{4a}R^{4b}, —NR^{4a}COR^{4b}, —NR^{4a}CONR^{4b}R^{4c}, —NR^{4a}CO₂R^{4b}, —NR^{4a}SONR^{4b}R^{4c}, —NR^{4a}SO₂NR^{4b}R^{4c}, or —NR^{4a}SO₂R^{4b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, —C₁₋₈alkyoxy, cycloalkyl, heterocyclyl, aryl, or heteroaryl;
R^{4a}, R^{4b}, and R^{4c} are each independently hydrogen, —C₁₋₈alkyl, —C₂₋₈alkenyl, —C₂₋₈alkynyl, cycloalkyl, heterocyclyl, aryl, or heteroaryl;
X¹ and X² are each independently a single bond, —CR^{X1}R^{X2}, —CR^{X1}R^{X2}CR^{X3}R^{X4}, or —CR^{X1}R^{X2}CR^{X3}R^{X4}CR^{X5}R^{X6};
wherein R^{X1}, R^{X2}, R^{X3}, R^{X4}, R^{X5} and R^{X6}, at each of its occurrence, are independently selected from hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl, 5- to 12-membered heteroaryl, each of said —C₁₋₈alkyl, —C₂₋₈alkenyl, —C₂₋₈alkynyl, C₃₋₈cycloalkyl, 3- to 8-membered heterocyclyl, C₆-C₁₂aryl or 5- to 12-membered heteroaryl is optionally substituted with at least one substituent R^{Xa};
each of said R^{Xa} is independently oxo, halogen, hydroxy, —C₁₋₈alkyl, —C₁₋₈alkoxy, —C₁₋₈alkyl, —C₂₋₈alkenyl, —C₂₋₈alkynyl, —C₁₋₈haloalkyl, —C₃-C₈cycloalkyl, 3- to 8-membered heterocyclyl, C₆-C₁₂aryl or 5- to 12-membered heteroaryl;
the Linker is a bond or a divalent linking group, and
the Degron is an E3 Ubiquitin ligase moiety.

2. The compound according to claim 1, wherein the Degron moiety is selected from: 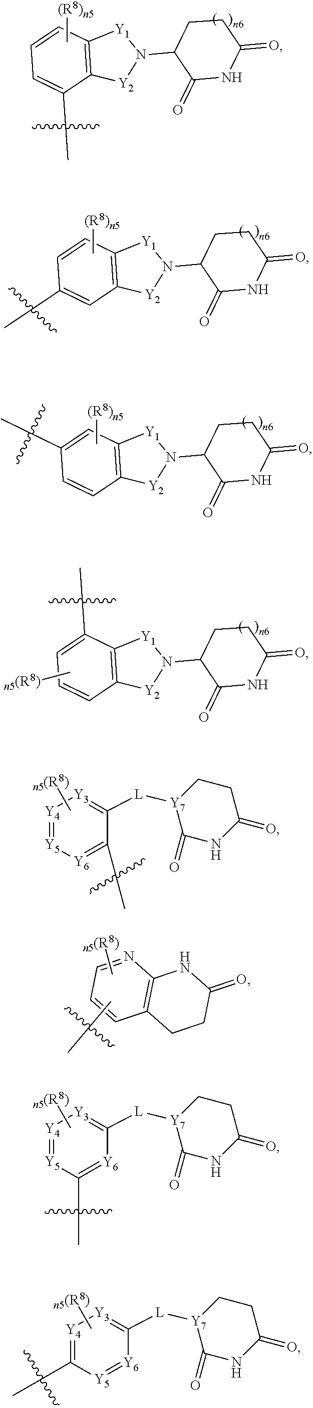 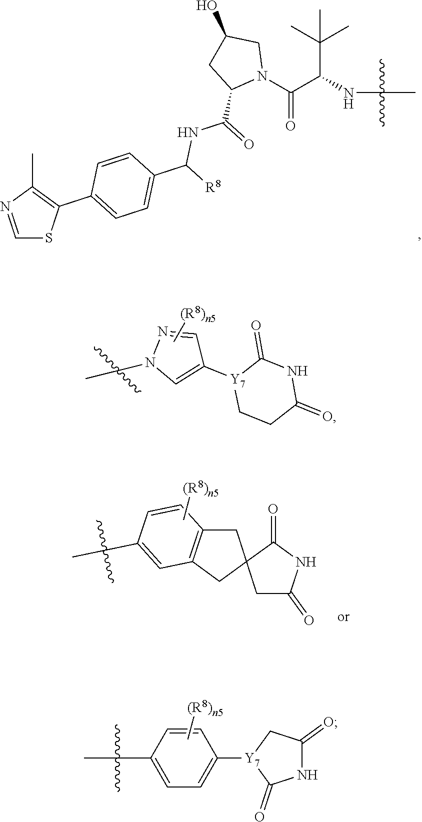
wherein
Y¹ and Y² are each independently —CH₂—, —NH— or —C(O)—;
Y³, Y⁴, Y⁵ and Y⁶ are each independently CH or N;
Y⁷ is CH or N;
L is selected from a bond, —CH₂—, —O—, —NH— and —S—;
n5 is 0, 1, 2, 3, or 4;
n6 is 0, 1, or 2;
R⁸ is each independently hydrogen, halogen, —C₁₋₈alkyl, —C₂₋₈alkenyl, —C₂₋₈alkynyl, cycloalkyl, heterocyclyl, aryl, heteroaryl, —CN, —NO₂, —OR^{8a}, —SO₂R^{8a}, —COR^{8a}, —CO₂R^{8a}, —CONR^{8a}R^{8b}, —C(═NR^{8a})NR^{8a}R^{8c}, —NR^{8a}R^{8b}, —NR^{8a}COR^{8b}, —NR^{8a}CONR^{8b}R^{8c}, —NR^{8a}CO₂R^{8b}, —NR^{8a}SONR^{8b}R^{8c}, —NR^{8a}SO₂NR^{8b}R^{8c}, or —NR^{8a}SO₂R^{8b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, —C₁₋₈alkyoxy, cycloalkyl, heterocyclyl, aryl, or heteroaryl; and
R^{8a}, R^{8b}, and R^{8c} are each independently hydrogen, —C₁₋₈alkyl, —C₂₋₈alkenyl, —C₂₋₈alkynyl, cycloalkyl, heterocyclyl, aryl, or heteroaryl.

3. The compound according to claim 2, wherein Degron moiety is selected from 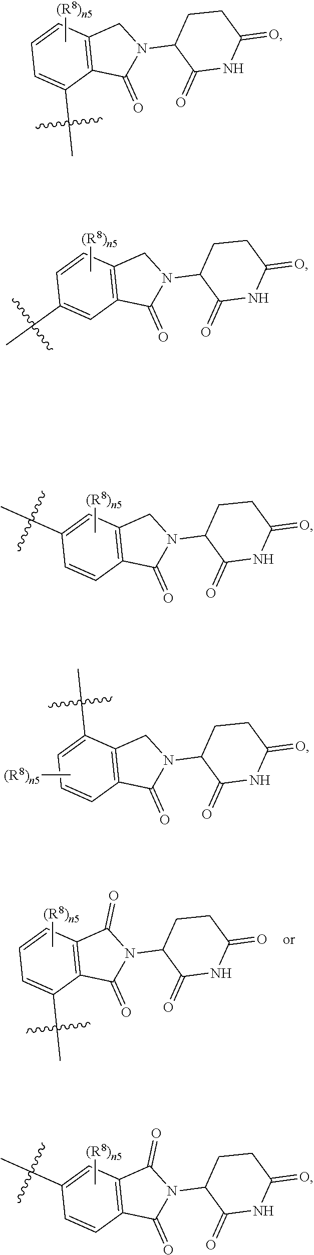
wherein R⁸ and n5 are defined as above.

4. The compound according to claim 2, wherein Degron moiety is selected from

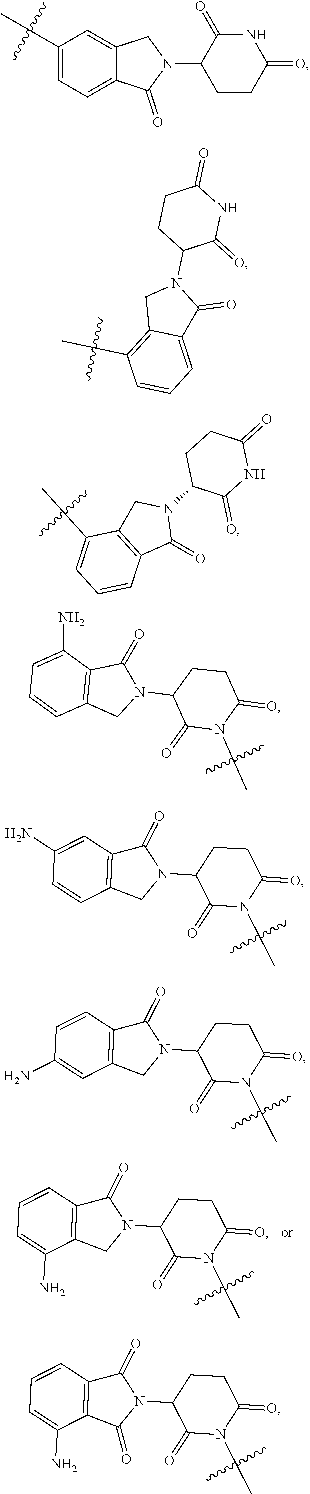

5. The compound according to claim 2, wherein Degron moiety is selected from 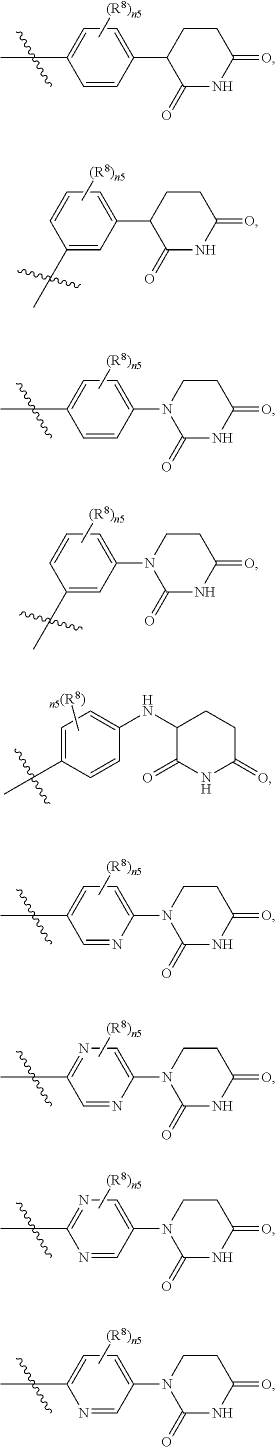 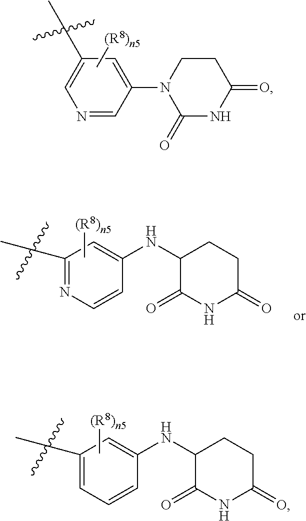
wherein R⁸ and n5 are defined as above.

6. The compound according to claim 2, wherein Degron moiety is selected from

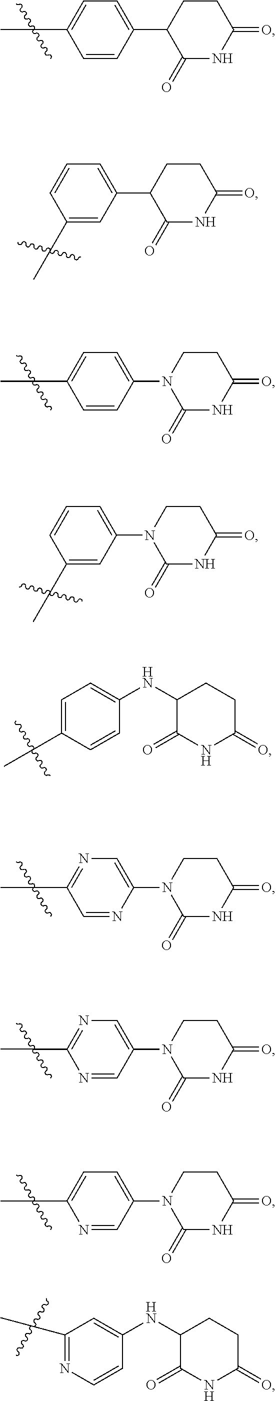

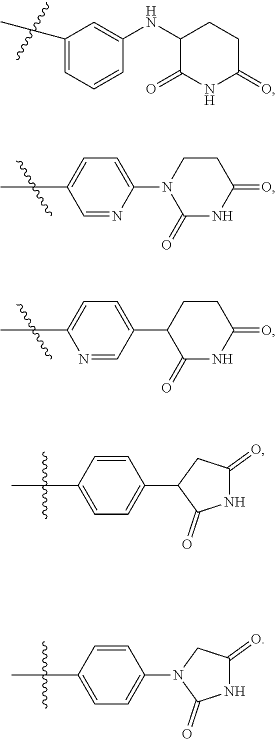 [SMILES: *c1cccc(NC2CCC(=O)NC2=O)c1 ; *c1ccc(N2CCC(=O)NC2=O)nc1 ; *c1ccc(C2CCC(=O)NC2=O)cn1 ; *c1ccc(C2CC(=O)NC2=O)cc1 ; *c1ccc(N2CC(=O)NC2=O)cc1]

7. The compound according to claim 2, wherein R⁸ is hydrogen, —F, —Cl, —Br, —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —CN, —NO₂, —OR^{8a}, —SO₂R^{8a}, —COR^{8a}, —CO₂R^{8a}, —CONR^{8a}R^{8b}, —C(═NR^{8a})NR^{8b}R^{8c}, —NR^{8a}R^{8b}, —NR^{8a}COR^{8b}, —NR^{8a}CONR^{8b}R^{8c}, —NR^{8a}CO₂R^{8b}, —NR^{8a}SONR^{8b}R^{8c}, —NR^{8a}SO₂NR^{8b}R^{8c}, or —NR^{8a}SO₂R^{8b}, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with —F, —Cl, —Br, —I, hydroxy, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl; and
R^{8a}, R^{8b}, and R^{8c} are each independently hydrogen, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl.

8. The compound according to claim 2, wherein R⁸ is hydrogen, —F, —Cl, —Br, —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl or —CN.

9. The compound according to claim 2, wherein R⁸ is hydrogen, —F, —Cl, —Br, —I, methyl, ethyl, propyl, butyl, pentyl, hexyl, cyclopropyl, cyclobutyl, cyclopentyl, cyclohexyl.

10. The compound according to claim 1, wherein the 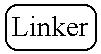
moiety is 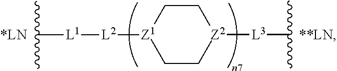
wherein *^{LN} refers to the position attached to the 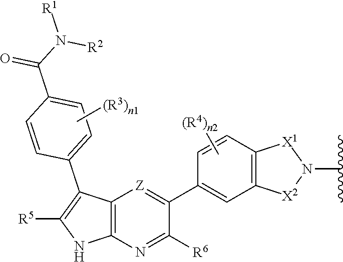
moiety, and **^{LN} refers to the position attached to the 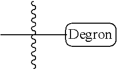
moiety;
Z¹ and Z² are each independently selected from CH or N;
n7 is 0 or 1;
L¹ is a single bond, —O—, —SO₂—, —C(O)—, —NR^{L1a}, —C₃-C₈cycloalkylene-, *^{L1}—O—C₁₋₈alkylene-**^{L1}, *^{L1}—C₁₋₈alkylene-O—**^{L1}, *^{L1}—SO₂—C₁₋₈alkylene-**^{L1}, *^{L1}—C₁₋₈alkylene-SO₂—**^{L1}, *^{L1}—C(O)—C₁₋₈alkylene-**^{L1}, *^{L1}—C₁₋₈alkylene-C(O)—**^{L1}, *^{L1}—NR^{L1a}—C₁₋₈alkylene-**^{L1}, *^{L1}—C₁₋₈alkylene-NR^{L1a}—**^{L1}, *^{L1}—NR^{L1a}C(O)—**^{L1}, *^{L1}—C(O)NR^{L1a}—**^{L1}, *^{L1}—NR^{L1a}C(O)C₁₋₈alkylene-**^{L1}, *^{L1}—C(O)NR^{L1a}C₁₋₈alkylene-**^{L1}, *^{L1}—C₁₋₈alkyleneNR^{L1a}C(O)—**^{L1}, *^{L1}—C₁₋₈alkyleneC(O)NR^{L1a}—**^{L1}, —C₁₋₈alkylene-, —C₂₋₈alkenylene-, —C₂₋₈alkynylene-, —[O(CR^{L1a}R^{L1b})ᵤ₁]ᵥ₁—, *^{L1}—CO—(CR^{L1a}R^{L1b})ᵤ₁—**^{L1}, *^{L1}—(CR^{L1a}R^{L1b})ᵤ₁—CO—**^{L1}, *^{L1}—(CR^{L1a}R^{L1b})ᵤ₁—**^{L1}; each of said —C₃-C₈cycloalkylene-, *^{L1}—O—C₁₋₈alkylene-**^{L1}, *^{L1}—C₁₋₈alkylene-O—**^{L1}, *^{L1}—SO₂—C₁₋₈alkylene-**^{L1}, *^{L1}—C₁₋₈alkylene-SO₂—**^{L1}, *^{L1}—C(O)—C₁₋₈alkylene-**^{L1}, *^{L1}—C₁₋₈alkylene-C(O)—**^{L1}, *^{L1}—NR^{L1a}—C₁₋₈alkylene-**^{L1}, *^{L1}—C₁₋₈alkylene-NR^{L1a}—**^{L1}, *^{L1}—NR^{L1a}C(O)C₁₋₈alkylene-**^{L1}, *^{L1}—C(O)NR^{L1a}C₁₋₈alkylene-**^{L1}, *^{L1}—C₁₋₈alkyleneNR^{L1a}C(O)—**^{L1}, *^{L1}—C₁₋₈alkyleneC(O)NR^{L1a}—**^{L1}, —C₁₋₈alkylene-, —C₂₋₈alkenylene or —C₂₋₈alkynylene- are optionally substituted with at least one RLIc;
u1 and v1 are each independently selected from 1, 2, 3, 4, 5, 6, 7 or 8;
wherein *^{L1} refers to the position attached to the 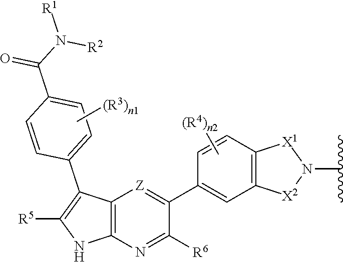
moiety, and
**^{L1} refers to the position attached to the 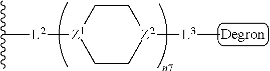
moiety;
L² is selected from a single bond, —O—, —SO₂—, —CO—, —NR^{L2a}—, —C₃-C₈cycloalkylene-, *^{L2}—O—C₁₋₈alkylene-**^{L2}, *^{L2}—C₁₋₈alkylene-O—**^{L2}, *^{L2}—SO₂—C₁₋₈alkylene-**^{L2}, *^{L2}—C₁₋₈alkylene-SO₂—**^{L2}, *^{L2}—CO—C₁₋₈alkylene-**^{L2}, *^{L2}—C₁₋₈alkylene-CO—**^{L2}, *^{L2}—NR^{L2a}—C₁₋₈alkylene-**^{L2}, *^{L2}—C₁₋₈alkylene-NR^{L2a}—**^{L2}, *^{L2}—NR^{L2a}C(O)—**^{L2}, *^{L2}—C(O)NR^{L2a}—**^{L2}, *^{L2}—NR^{L2a}C(O)C₁₋₈alkylene-**^{L2}, *^{L2}—C(O)NR^{L2a}C₁₋₈alkylene-**^{L2}, *^{L2}—C₁₋₈alkyleneNR^{L2a}C(O)—**^{L2}, *^{L2}—C₁₋₈alkyleneC(O)NR^{L2a}—**^{L2}, —C₁₋₈alkylene-, —C₂₋₈alkenylene-, —C₂₋₈alkynylene- , —[O(CR^{L2a}R^{L2b})ᵤ₂]ᵥ₂—, *^{L2}—CO—(CR^{L2a}R^{L2b})ᵤ₂—**^{L2}, *^{L2}—(CR^{L2a}R^{L2b})ᵤ₂—CO—**^{L2}, *^{L2}—(CR^{L2a}R^{L2b})ᵤ₂—**^{L2}; each of said —C₃-C₈cycloalkylene-, *^{L2}—O—C₁₋₈alkylene-**^{L2}, *^{L2}—C₁₋₈alkylene-O—**^{L2}, *^{L2}—SO₂—C₁₋₈alkylene-**^{L2}, *^{L2}—C₁₋₈alkylene-SO₂—**^{L2}, *^{L2}—CO—C₁₋₈alkylene-**^{L2}, *^{L2}—C₁₋₈alkylene-CO—**^{L2}, *^{L2}—NR^{L2a}—C₁₋₈alkylene-**^{L2}, *^{L2}—C₁₋₈alkylene-NR^{L2a}—**^{L2}, *^{L2}—NR^{L2a}C(O)C₁₋₈alkylene-**^{L2}, *^{L2}—C(O)NR^{L2a}C₁₋₈alkylene-**^{L2}, *^{L2}—C₁₋₈alkyleneNR^{L2a}C(O)—**^{L2}, *^{L2}—C₁₋₈alkyleneC(O)NR^{L2a}—**^{L2}, —C₁₋₈alkylene-, —C₂₋₈alkenylene or —C₂₋₈alkynylene- are optionally substituted with at least one substituent R^{L2c};
u2 and v2 are each independently selected from 1, 2, 3, 4, 5, 6, 7 or 8;
wherein *^{L2} refers to the position attached to the 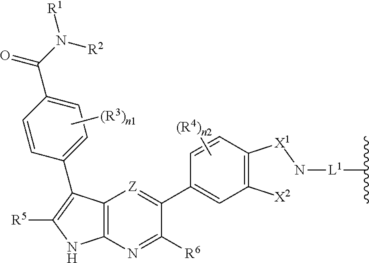
moiety, and
**^{L2} refers to the position attached to the 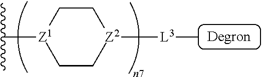
moiety;
L₃ is selected from a single bond, —O—, —SO₂—, —CO—, —NR^{L3a}—, —C₃-C₈cycloalkylene-, *^{L3}—O—C₁₋₈alkylene-**^{L3}, *^{L3}—C₁₋₈alkylene-O—**^{L3}, *^{L3}—SO₂—C₁₋₈alkylene-**^{L3}, *^{L3}—C₁₋₈alkylene-SO₂—**^{L3}, *^{L3}—CO—C₁₋₈alkylene-**^{L3}, *^{L3}—C₁₋₈alkylene-CO—**^{L3}, *^{L3}—NR^{L3a}—C₁₋₈alkylene-**^{L3}, *^{L3}—C₁₋₈alkylene-NR^{L3a}—**^{L3}, *^{L3}—NR^{L3a}C(O)—**^{L3}, *^{L3}—C(O)NR^{L3a}—**^{L3}, *^{L3}—NR^{L3a}C(O)C₁₋₈alkylene-**^{L3}, *^{L3}—C(O)NR^{L3a}C₁₋₈alkylene-**^{L3}, *^{L3}—C₁₋₈alkyleneNR^{L3a}C(O)—**^{L3}, *^{L3}—C₁₋₈alkyleneC(O)NR^{L3a}—**^{L3}, —C₁₋₈alkylene-, —C₂₋₈alkenylene-, —C₂₋₈alkynylene-, —[O(CR^{L3a}R^{L3b})ᵤ₃]ᵥ₃—, —*^{L3}—CO—(CR^{L3a}R^{L3b})ᵤ₃—**^{L3}, *^{L3}—(CR^{L3a}R^{L3b})ᵤ₃—CO—**^{L3}, *^{L3}—(CR^{L3a}R^{L3b})ᵤ₃—**^{L3}; each of said —C₃-C₈cycloalkylene-, *^{L3}—O—C₁₋₈alkylene-**^{L3}, *^{L3}—C₁₋₈alkylene-O—**^{L3}, *^{L3}—SO₂—C₁₋₈alkylene-**^{L3}, *^{L3}—C₁₋₈alkylene-SO₂—**^{L3}, *^{L3}—CO—C₁₋₈alkylene-**^{L3}, *^{L3}—C₁₋₈alkylene-CO—**^{L3}, *^{L}—NR^{L3a}—C₁₋₈alkylene-**^{L3}, *^{L3}—C₁₋₈alkylene-NR^{L3a}—**^{L3}, *^{L3}—NR^{L3a}C(O)C₁₋₈alkylene-**^{L3}, *^{L3}—C(O)NR^{L3a}C₁₋₈alkylene-**^{L3}, *^{L3}—C₁₋₈alkyleneNR^{L3a}C(O)—**^{L3}, *^{L3}—C₁₋₈alkyleneC(O)NR^{L3a}—**^{L3}, —C₁₋₈alkylene-, —C₂₋₈alkenylene-, —C₂₋₈alkynylene-, are optionally substituted with at least one substituent R^{L3c};
u3 and v3 are each independently selected from 1, 2, 3, 4, 5, 6, 7 or 8;
wherein *^{L3} refers to the position attached to the 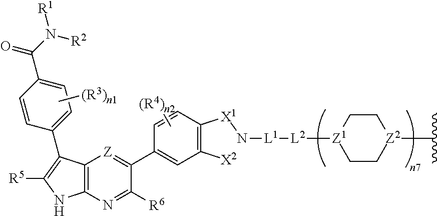
moiety, and **^{L3} referes to the position attached to the 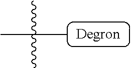
moiety;
R^{L1a}, R^{L1b}, R^{L1c}, RL^{2a}, R^{L2b}, R^{L2c}, R^{L3a}, R^{L3b}, R^{L3c} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or membered heteroaryl is optionally substituted with at least one substituent R^{L3d}; R^{L3d} is halogen, hydroxy, —C₁₋₈alkyl, -haloC₁₋₈alkyl, —C₁₋₈alkoxy, —C₂₋₈alkenyl, —C₂₋₈alkynyl, cycloalkyl, heterocyclyl, aryl or heteroaryl.

11. The compound according to claims 10, wherein L¹ is a single bond, —O—, —SO₂—, —C(O)—, —NH—, —N(CH₃)—, —N(C₂H₅)—, —N(C₃H₇)—, *^{L1}—O—CH₂—**^{L1}, *^{L1}—O—C₂H₄ —**^{L1}, *^{L1}—O—C₃H₆—**^{L1}, *^{L1}—O—C₄H₈—**^{L1}, *^{L1}—CH₂—O—**^{L1}, *^{L1}—C₂H₄—O—**^{L1}, *^{L1}—C₃H₆—O—**^{L1}, *^{L1}—C₄H₈—O—**^{L1}, *^{L1}—SO₂—CH₂—**^{L1}, *^{L1}13 SO₂—C₂H₄ —**^{L1}, *^{L1}—SO₂—C₃H₆—**^{L1}, *^{L1}—SO₂—C₄H₈—**^{L1}, *^{L1}—CH₂—SO₂—**^{L1}, *^{L1}—C₂H₄—SO₂—**^{L1}, *^{L1}—C₃H₆—SO₂—**^{L1}, *^{L1}—C₄H₈—SO₂—**^{L1}, *^{L1}—C(O)—CH₂—**^{L1}, *^{L1}—C(O)—C₂H₄—**^{L1}, *^{L1}—C(O)—C₃H₆—**^{L1}, *^{L1}—C(O)—C₄H₈—**^{L1}, *^{L1}—CH₂—C(O)—**^{L1}, *^{L1}—C₂H₄—C(O)—**^{L1}, *^{L1}—C₃H₆—C(O)—**^{L1}, *^{L1}—C₄H₈—C(O)—**^{L1}, *^{L1}—NH—CH₂—**^{L1}, *^{L1}—NH—C₂H₄—**^{L1}, *^{L1}—NH—C₃H₆—**^{L1}, *^{L1}—NH—C₄H₈—**^{L1}, *^{L1}—CH₂—NH—**^{L1}, *^{L1}—C₂H₄—NH—**^{L1}, *^{L1}—C₃H₆—NH—**^{L1}, *^{L1}—C₄H₈—NH—**^{L1}, *^{L1}—NHC(O)—**^{L1}, *^{L1}—C(O)NH—**^{L1}, *^{L1}—N(CH₃)C(O)—**^{L1}, *^{L1}—C(O)N(CH₃)—**^{L1}, *^{L1}—N(C₂H₅)C(O)—**^{L1}, *^{L1}—C(O)N(C₂H₅)—**^{L1}, *^{L1}—N(C₃H₇)C(O)—**^{L1}, *^{L1}—C(O)N(C₃H₇)—**^{L1}, *^{L1}—NHC(O)CH₂—**^{L1}, *^{L1}—NHC(O)C₂H₄—**^{L1}, *^{L1}—NHC(O)C₃H₆—**^{L1}, *^{L1}—NHC(O)C₄H₈—**^{L1}, *^{L1}—C(O)NHCH₂—**^{L1}, *^{L1}—C(O)NHC₂H₄—**^{L1}, *^{L1}—C(O)NHC₃H₆—**^{L1}, *^{L1}—C(O)NHC₄H₈—**^{L1}, *^{L1}—CH₂NHC(O)—**^{L1}, *^{L1}—C₂H₄NHC(O)—**^{L1}, *^{L1}—C₃H₆NHC(O)—**^{L1}, *^{L1}—C₄H₈NHC(O)—**^{L1}, *^{L1}—CH₂C(O)NH—**^{L1}, *^{L1}—C₂H₄C(O)NH—**^{L1}, *^{L1}—C₃H₆C(O)NH—**^{L1}, *^{L1}—C₄H₈C(O)NH—**^{L1}, —CH₂—, —C₂H₄—, —C₃H₆—, —C₄H₈—, —O(CH₂)₂—, —[O(CH₂)₂]₂—, —[O(CH₂)₂]₃—, —[O(CH₂)₂]₄— or —[O(CH₂)₂]₅—.

12. The compound according to claims 10, wherein L¹ is *^{L1}—CH₂—C(O)—**^{L1}, *^{L1}—CH₂CH₂—C(O)—**^{L1}, *^{L1}—CH₂CH₂CH₂—C(O)—**^{L1}, *^{L1}—CH₂—NH—**^{L1}, *^{L1}—CH₂CH₂—NH—**^{L1}, *^{L1}—CH₂CH₂CH₂—NH—**^{L1}, *^{L1}—CH₂NHC(O)—**^{L1}, *^{L1}—CH₂CH₂NHC(O)—**^{L1}, *^{L1}—CH₂CH₂CH₂NHC(O)—**^{L1}, *^{L1}—CH₂C(O)NH—**^{L1}, *^{L1}—CH₂CH₂C(O)NH—**^{L1}, *^{L1}—CH₂CH₂CH₂C(O)NH—**^{L1}, —CH₂—, —CH₂CH₂—, —CH₂CH₂CH₂—.

13. The compound according to claims 10, wherein L² is a single bond, —O—, —SO₂—, —C(O)—, —NH—, —N(CH₃)—, —N(C₂H₅)—, —N(C₃H₇)—, *^{L2}—O—CH₂—**^{L2}, *^{L2}—O—C₂H₄—**^{L2}, *^{L2}—O—C₃H₆—**^{L2}, *^{L2}—O—C₄H₈—**^{L2}, *^{L2}—CH₂—O—**^{L2}, *^{L2}—C₂H₄—O—**^{L2}, *^{L2}—C₃H₆—O—**^{L2}, *^{L2}—C₄H₈—O—**^{L2}, *^{L2}—SO₂—CH₂—**^{L2}, *^{L2}—SO₂—C₂H₄—**^{L2}, *^{L2}—SO₂—C₃H₆—**^{L2}, *^{L2}—SO₂—C₄H₈—**^{L2}, *^{L2}—CH₂—SO₂—**^{L2}, *^{L2}—C₂H₄—SO₂—**^{L2}, *^{L2}—C₃H₆—SO₂—**^{L2}, *^{L2}—C₄H₈—SO₂—**^{L2}, *^{L2}—C(O)—CH₂—**^{L2}, *^{L2}—C(O)—C₂H₄—**^{L2}, *^{L2}—C(O)—C₃H₆—**^{L2}, *^{L2}—C(O)—C₄H₈—**^{L2}, *^{L2}—CH₂—C(O)—**^{L2}, *^{L2}—C₂H₄—C(O)—**^{L2}, *^{L2}—C₃H₆—C(O)—**^{L2}, *^{L2}—C₄H8—C(O)—**^{L2}, *^{L2}—NH—CH₂—**^{L2}, *^{L2}—NH—C₂H₄—**^{L2}, *^{L2}—NH—C₃H₆—**^{L2}, *^{L2}—NH—C₄H₈—**^{L2}, *^{L2}—CH₂—NH—**^{L2}, *^{L2}—C₂H₄—NH—**^{L2}, *^{L2}—C₃H₆—NH—**^{L2}, *^{L2}—C₄H₈—NH—**^{L2}, *^{L2}—NHC(O)—**^{L2}, *^{L2}—C(O)NH—**^{L2}, *^{L2}—N(CH₃)C(O)—**^{L2}, *^{L2}—C(O)N(CH₃)—**^{L2}, *^{L2}—N(C₂H₅)C(O)—**^{L2}, *^{L2}—C(O)N(C₂H₅)—**^{L2}, *^{L2}—N(C₃H₇)C(O)—**^{L2}, *^{L2}—C(O)N(C₃H₇)—**^{L2}, *^{L2}—NHC(O)CH₂—**^{L2}, *^{L2}—NHC(O)C₂H₄—**^{L2}, *^{L2}—NHC(O)C₃H₆—**^{L2}, *^{L2}—NHC(O)C₄H₈—**^{L2}, *^{L2}—C(O)NHCH₂—**^{L2}, *^{L2}—C(O)NHC₂H₄—**^{L2}, *^{L2}—C(O)NHC₃H₆—**^{L2}, *^{L2}—C(O)NHC₄H₈—**^{L2}, *^{L2}—CH₂NHC(O)—**^{L2}, *^{L2}—C₂H₄NHC(O)—**^{L2}, *^{L2}—C₃H(NHC(O)—**^{L2}, *^{L2}—C₄H₈NHC(O)—**^{L2}, *^{L2}—CH₂C(O)NH—**^{L2}, *^{L2}—C₂H₄C(O)NH—**^{L2}, *^{L2}—C₃H₆C(O)NH—**^{L2}, *^{L2}—C₄H₈C(O)NH—**^{L2}, —CH₂—, —C₂H₄—, —C₃H₆—, —C₄H₈—, —O(CH₂)₂—, —[O(CH₂)₂]₂—, —[O(CH₂)₂]₃—, —[O(CH₂)₂]₄—or —[O(CH₂)₂]₅—.

14. The compound according to claims 10, wherein L² is a single bond, —C(O)—, —NH—, *^{L2}—NHC(O)—**^{L2}, *^{L2}—C(O)NH—**^{L2}, *^{L2}—NH—CH₂—**^{L2}, *^{L2}—NH—CH₂CH₂—**^{L2}, *^{L2}—NH—CH₂CH₂CH₂—**^{L2}, *^{L2}—C(O)—CH₂—**^{L2}, *^{L2}—C(O)—CH₂CH₂—**^{L2}, *^{L2}—C(O)—CH₂CH₂CH₂—**^{L2}, *^{L2}—NHC(O)CH₂—**^{L2}, *^{L2}—NHC(O)CH₂CH₂—**^{L2}, *^{L2}—NHC(O)CH₂CH₂CH₂—**^{L2}, *^{L2}—C(O)NHCH₂—**^{L2}, *^{L2}—C(O)NHCH₂CH₂—**^{L2}, or *^{L2}—C(O)NHCH₂CH₂CH₂—**^{L2}.

15. The compound according to claims 10, wherein L³ is a single bond, —O—, —SO₂—, —C(O)—, —NH—, —N(CH₃)—, —N(C₂H₅)—, —N(C₃H₇)—, *^{L3}—O—CH₂—**^{L3}, *^{L3}—O—C₂H₄—**^{L3}, *^{L3}—O—C₃H₆—**^{L3}, *^{L3}—O—C₄H₈—**^{L3}, *^{L3}—CH₂—O—**^{L3}, *^{L3}—C₂H₄—O—**^{L3}, *^{L3}—C₃H₆—O—**^{L3}, *^{L}₃—C₄H₈—O—**^{L3}, *^{L3}—SO₂—CH₂—**^{L3}, *^{L3}—SO₂—C₂H₄—**^{L3}, *^{L3}—SO₂—C₃H₆—**^{L3}, *^{L3}—SO₂—C₄H₈—**^{L3}, *^{L3}—CH₂—SO₂—**^{L3}, *^{L3}—C₂H₄—SO₂—**^{L3}, *^{L3}—C₃H₆—SO₂—**^{L3}, *^{L3}—C₄H₈—SO₂—**^{L3}, *^{L3}—C(O)—CH₂—**^{L3}, *^{L3}—C(O)—C₂H₄—**^{L3}, *^{L3}—C(O)—C₃H₆—**^{L3}, *^{L3}—C(O)—C₄H₈—**^{L3}, *^{L3}—CH₂—C(O)—**^{L3}, *^{L3}—C₂H₄—C(O)—**^{L3}, *^{L3}—C₃H₆—C(O)—**^{L3}, *^{L3}—C₄H₈—C(O)—**^{L3}, *^{L3}—NH—CH₂—**^{L3}, *^{L3}—NH—C₂H₄—**^{L3}, *^{L3}—NH—C₃H₆—**^{L3}, *^{L3}—NH—C₄H₈—**^{L3}, *^{L3}—CH₂—NH—**^{L3}, *^{L3}—C₂H₄—NH—**^{L3}, *^{L3}—C₃H₆—NH—**^{L3}, *^{L3}—C₄H₈—NH—**^{L3}, *^{L3}—NHC(O)—**^{L3}, *^{L3}—C(O)NH—**^{L3}, *^{L3}—N(CH₃)C(O)—**^{L3}, *^{L3}—C(O)N(CH₃)—**^{L3}, *^{L3}—N(C₂H₅)C(O)—**^{L3}, *^{L3}—C(O)N(C₂H₅)—**^{L3}, *^{L3}—N(C₃H₇)C(O)—**^{L3}, *^{L3}—C(O)N(C₃H₇)—**^{L3}, *^{L3}—NHC(O)CH₂—**^{L3}, *^{L3}—NHC(O)C₂H₄—**^{L3}, *^{L3}—NHC(O)C₃H₆—**^{L3}, *^{L3}—NHC(O)C₄H₈—**^{L3}, *^{L3}—C(O)NHCH₂—**^{L3}, *^{L3}—C(O)NHC₂H₄—**^{L3}, *^{L3}—C(O)NHC₃H₆—**^{L3}, *^{L3}—C(O)NHC₄H₈—**^{L3}, *^{L3}CH₂NHC(O)—**^{L3}, *^{L3}—C₂H₄NHC(O)—**^{L3}, *^{L3}—C₃H₆NHC(O)—**^{L3}, *^{L3}—C₄H₈NHC(O)—**^{L3}, *^{L3}—CH₂C(O)NH—**^{L3}, *^{L3}—C₂H₄C(O)NH—**^{L3}, *^{L3}—C₃H₆C(O)NH—**^{L3}, *^{L3}—C₄H₈C(O)NH—**^{L3}, —CH₂—, —C₂H₄—, —C₃H₆—, —C₄H₈—, —O(CH₂)₂—, —[O(CH₂)₂]₂—, —[O(CH₂)₂]₃—, —[O(CH₂)₂]₄— or —[O(CH₂)₂]₅—.

16. The compound according to claims 10, wherein L³ is a single bond.

17. The compound according to claims 10, wherein 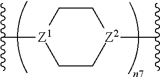
moiety is 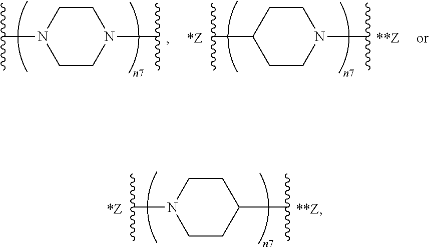
wherein *^{LN} refers to the position attached to the 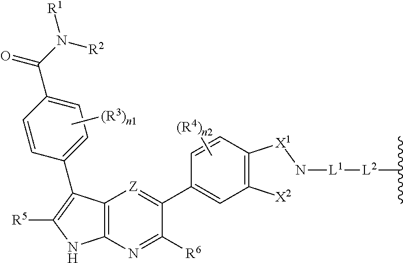
moiety, and **^{LN} refers to the position attached to the 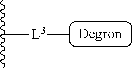
moiety.

18. The compound according to claim 1, wherein the 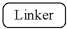
moiety is 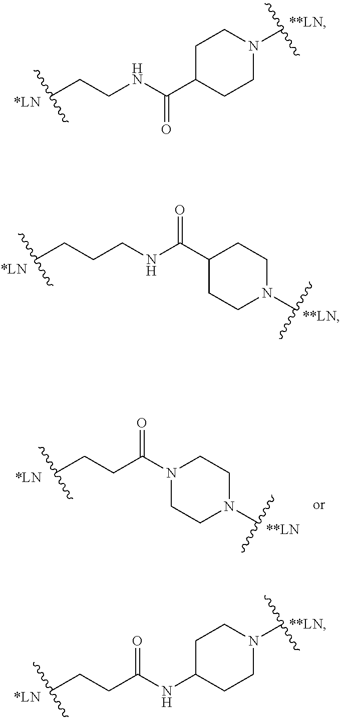 [SMILES: *CCNC(=O)C1CCN(*)CC1 ; *CCCNC(=O)C1CCN(*)CC1 ; *CCC(=O)N1CCN(*)CC1 ; *CCC(=O)NC1CCN(*)CC1]
wherein *^{LN} refers to the position attached to the 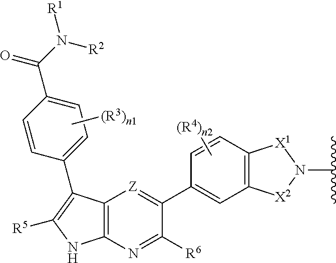
moiety, and **^{LN} refers to the position attached to the 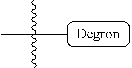
moiety.

19. The compound according to claim 1, wherein R¹ and R² are each independently hydrogen, deuterium, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one substituents R^{1a}; or
R¹ and R² together with the atom to which they are attached, form a 3-, 4-, 5-, 6-, 7- or 8-membered ring, said ring comprising 0, 1 or 2 additional heteroatoms independently selected from nitrogen, oxygen or optionally oxidized sulfur as ring member (s), said ring is optionally substituted with at least one substituents R^{1b}.
R^{1a} and R^{1b} are each independently —D, —F, —Cl, —Br, —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, oxo, —CN, —NO₂, —OR^{1c}, —SO₂R^{1c}, —SO₂NR^{1c}R^{1d}, —COR^{1c}, —CO₂R^{1c}, —CONR^{1c}R^{1d}, —C(═NR^{1c})NR^{1d}R^{1e}, —NR^{1c}R^{1d}, —NR^{1c}COR^{1d}, —NR^{1c}CONR^{1d}R^{1e}, —NR^{1c}CO₂R^{1c}, —NR^{1c}SONR^{1c}R^{1d}, —NR^{1c}SO₂NR^{1d}R^{1e}, or —NR^{1c}SO₂R^{1d}, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one substituents selected from —F, —Cl, —Br, —I, methyl, ethyl, propyl, butyl, pentyl, hexyl, heptyl, octyl, —OR^{1f}, —NR^{1f}R^{1g}, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl;
R^{1c}, R^{1d}, R^{1e}, R^{1f}, and R^{1g} are each independently hydrogen, methyl, ethyl, propyl, butyl, pentyl, hexyl, heptyl, octyl, C₁₋₈alkoxy-C₁₋₈alkyl-, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl.

20. The compound according to claim 1, wherein R¹ and R² are each independently hydrogen, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl; each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one OH; or
R¹ and R² together with the atom to which they are attached, form a 3-, 4-, 5-, 6-, 7- or 8-membered ring, said ring comprising 0, 1 or 2 additional heteroatoms independently selected from nitrogen, oxygen or optionally oxidized sulfur as ring member (s).

21. The compound according to claim 1, wherein R¹ and R² are each independently hydrogen, —CD₃, methyl, ethyl, n-propyl, iso-propyl, n-butyl, sec-butyl, iso-butyl, tert-butyl, 2-hydroxypropyl, hydroxymethyl, 1-hydroxyethyl, 2-hydroxyethyl, 1-hydroxypropyl, 3-hydroxypropyl, 1-hydroxybutyl, 2-hydroxybutyl, 3-hydroxybutyl, or 4-hydroxybutyl.

22. The compound according to claim 1, wherein R³, at each of its occurrence, are independently —F, —Cl, —Br, —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —CH₂-heterocyclyl, —CH₂CH₂-heterocyclyl, —CH₂CH₂CH₂-heterocyclyl, —CH₂CH₂CH₂CH₂-heterocyclyl, —CH₂-cycloalkyl, —CH CH₂-cycloalkyl, —CH CH₂CH₂-cycloalkyl, —CH₂CH₂CH₂CH₂-cycloalkyl, oxo, —CN, —NO₂, —OR^{3a}, —SO₂R^{3a}, —SO₂NR^{3a}R^{3b}, —COR^{3a}, —CO₂R^{3a}, —CONR^{3a}R^{3b}, —C(═NR^{3a})NR^{3b}R^{3c}, —NR^{3a}R^{3b}, —NR^{3a}COR^{3b}, —NR^{3a}CONR^{3b}R^{3c}, —NR^{3a}CO₂R^{3b}, —NR^{3a}SONR^{3b}R^{3c}, —NR^{3a}SO₂NR^{3b}R^{3c}, or —NR^{3a}SO₂R^{3h}, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —CH₂-heterocyclyl, —CH₂CH₂-heterocyclyl, —CH₂CH₂CH₂-heterocyclyl, —CH₂CH₂CH₂CH₂-heterocyclyl, —CH₂-cycloalkyl, —CH₂CH₂-cycloalkyl, —CH₂CH₂CH₂-cycloalkyl or —CH₂CH₂CH₂CH₂-cycloalkyl is optionally substituted with at least one substituents R^{3d}; or
or two R³, when on adjacent carbon atoms of the phenyl ring, together with the two intervening carbon atoms to which they are attached, form a 5- to 8-membered ring comprising 0, 1 or 2 heteroatoms independently selected from nitrogen, oxygen or optionally oxidized sulfur as ring member (s);
R^{3a}, R^{3b}, and R^{3a} are each independently hydrogen, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl, each of said methyl, ethyl, propyl, butyl, pentyl, hexyl, heptyl, octyl, vinyl, ethynyl. cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one substituents R^{3e}; or
(R^{3a} and R^{3b}), (R^{3b} and R^{3c}), or (R^{3c} and R^{3a}), together with the atom (s) to which they are attached, form a 3- to 12-membered ring, said ring comprising 0, 1 or 2 additional heteroatoms independently selected from nitrogen, oxygen or optionally oxidized sulfur as ring member (s), said ring is optionally substituted with at least one substituents R^{3e};
R^{3d} and R^{3e} are each independently —F, —Cl, —Br, —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, oxo, —CN, —NO₂, —OR^{3f}, —SO₂R^{3f}, —SO₂NR^{3f}R^{3g}, —COR^{3f}, —CO₂R^{3f}, —CONR^{3f}R^{3g}, —C(═NR^{3f})NRR^{3g}R^{3h}, —NR^{3f}R^{3g}, —NR^{3f}COR^{3g}, —NR^{3f}CONR^{3g}R^{3h}, —NR^{3f}CO₂R^{3f}, —NR^{3f}SONR^{3f}R^{3g}, —NR^{3f}SO₂NR^{3g}R^{3h}, or —NR^{3f}SO₂R^{3g}, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one substituents selected from —F, —Cl, —Br, —I, methyl, ethyl, propyl, butyl, pentyl, hexyl, heptyl, octyl, —OR³ⁱ, —NR³ⁱR^{3j}, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl;
R^{3f}, R^{3g}, R^{3h}, R³ⁱ, and R^{3j} are each independently hydrogen, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, C₁₋₈alkoxy-C₁₋₈alkyl-

23. The compound according to claim 1, wherein R³, at each of its occurrence, are independently —F, —Cl, —F, —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —CH₂-heterocyclyl, —CH₂CH₂-heterocyclyl, —CH₂CH₂CH₂-heterocyclyl, —CH₂CH₂CH₂CH₂-heterocyclyl, —CH₂-cycloalkyl, —CH₂CH₂-cycloalkyl, —CH₂CH₂CH₂-cycloalkyl, —CH₂CH₂CH₂CH₂-cycloalkyl, oxo, —CN, —NO₂, —OR^{3a}, —SO₂R^{3a}, —SO₂NR^{3a}R^{3b}, —COR^{3a}, —CO₂R^{3a}, —CONR^{3a}R^{3b}, —C(═NR^{3a})NR^{3b}R^{3c}, —NR^{3a}R^{3b}, —NR^{3a}COR^{3b}, —NR^{3a}CONR^{3b}R^{3c}, —NR^{3a}CO₂ R^{3b}, —NR^{3a}SONR^{3b}R^{3c}, —NR^{3a}SO₂NR^{3b}R^{3c}, or —NR^{3a}SO₂R^{3b}, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —CH₂-heterocyclyl, —CH₂CH₂-heterocyclyl, —CH₂CH₂CH₂-heterocyclyl, —CH₂CH₂CH₂CH₂-heterocyclyl, —CH₂-cycloalkyl, —CH₂CH₂-cycloalkyl, —CH₂CH₂CH₂-cycloalkyl or —CH₂CH₂CH₂CH₂-cycloalkyl is optionally substituted with at least one substituents R^{3d}; or
two R^{3a}, when on adjacent carbon atoms of the phenyl ring, together with the two intervening carbon atoms to which they are attached, form a 5- to 8-membered ring comprising 0, 1 or 2 heteroatoms independently selected from nitrogen, oxygen or optionally oxidized sulfur as ring member (s);
R^{3a}, R^{3b}, and R^{3c} are each independently hydrogen, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one substituents R^{3e}; or
(R^{3a} and R^{3b}), (R^{3b} and R^{3c}), or (R^{3c} and R^{3a}), together with the atom (s) to which they are attached, form a 3- to 12-membered ring, said ring comprising 0, 1 or 2 additional heteroatoms independently selected from nitrogen, oxygen or optionally oxidized sulfur as ring member (s), said ring is optionally substituted with at least one substituents R^{3e};
R^{3d} and R^{3e} are each independently —F, —Cl, —Br, —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, oxo, —CN, —NO₂, —OR^{3f}, —SO₂R^{3f}, —SO₂NR^{3f}R^{3g}, —COR^{3f}, —CO₂R^{3f}, —CONR^{3f}R^{3g}, —C(═NR^{3f})NR^{3g}R^{3h}, —NR^{3f}R^{3g}, —NR^{3f}COR^{3g}, —NR^{3f}CONR^{3g}R^{3h}, —NR^{3f}CO₂R^{3f}, —NR^{3f}SONR^{3f}R^{3g}, —NR^{3f}SO₂NR^{3g}R^{3h}, or —NR^{3f}SO₂R^{3g}, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one substituents selected from —F, —Cl, —Br, —I, methyl, ethyl, propyl, butyl, pentyl, hexyl, heptyl, octyl, —OR³ⁱ, —NR³ⁱR^{3j}, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl;
R^{3f}, R^{3g}, R^{3h}, R³ⁱ, and R^{3j} are each independently hydrogen, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, C₁₋₈alkoxy-C₁₋₈alkyl.

23. The compound according to claim 1, wherein R³, at each of its occurrence, is selected from methyl, ethyl, propyl, butyl, pentyl, hexyl, heptyl, octyl, —F, —Cl, —Br, —I, —CN, OR^{3a} or —NR^{3a}CONR^{3b}R^{3c}; said—C₁₋₈alkyl is optionally substituted with at least one substituents R^{3d}; R^{3a}, R^{3b} and R^{3c} are each independently hydrogen, or —C₁₋₈alkyl (preferably methyl); R^{3d} is each independently —F, —Cl, —Br, —I, methyl, ethyl, propyl, butyl, pentyl, hexyl, heptyl or octyl.

24. The compound according to claim 1, wherein R³ is —F, —Cl, —Br, —I, OH, CN, —CH₃, —C₂H₅, —CH₂CH₂CH₃, —CH(CH₃)CH₃, —CH₂CH₂CH₂CH₃, —CH(CH₃)CH₂CH₃, —CH₂CH(CH₃)CH₃, —C(CH₃)₃, —CH₂F, —CHF₂, —CF₃ or —NHCOCH₃.

25. The compound according to claim 1, wherein

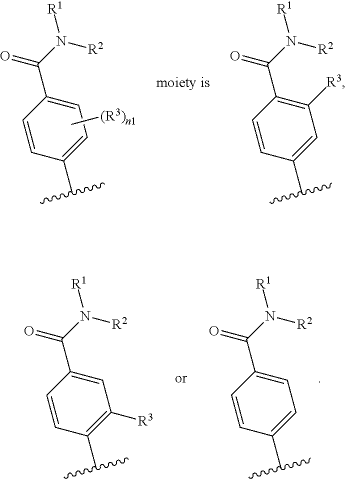

26. The compound according to claim 1, wherein 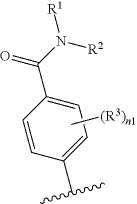
moiety is

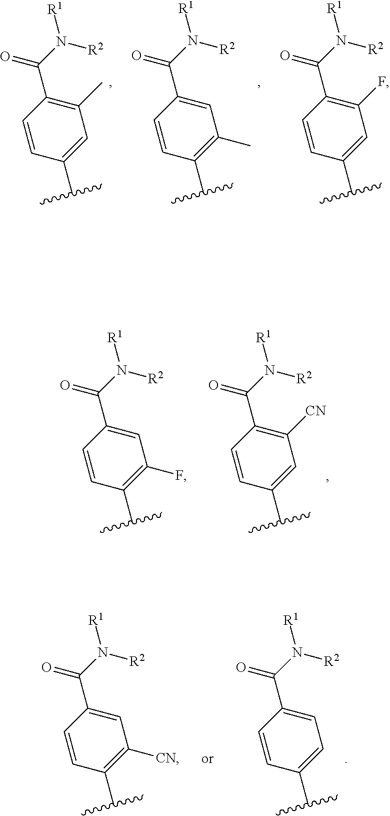

27. The compound according to claim 1, wherein R⁴, R⁵ and R⁶ are each independently hydrogen, —F, —Cl, —Br, —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, oxo, —CN, —NO₂, —OR^{4a}, —SO₂R^{4a}, —COR^{4a}, —CO₂R^{4a}, —CONR^{4a}R^{4b}, —C(═NR^{4a})NR^{4b}R^{4c}, —NR^{4a}R^{4b}, —NR^{4a}COR^{4b}, —NR^{4a}CONR^{4b}R^{4c}, —NR^{4a}CO₂R^{4b}, —NR^{4a}SONR^{4b}R^{4c}, —NR^{4a}SO₂NR_{4b}R^{4c}, or —NR^{4a}SO₂R^{4b}, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with —F, —Cl, —Br, —I, hydroxy, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl;
R^{4a}, R^{4b}, and R^{4c} are each independently hydrogen, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyraziny.

28. The compound according to claim 1, wherein R⁵ and R⁶ are each independently hydrogen or —C₁₋₈alkyl (preferably hydrogen or methyl).

29. The compound according to claim 1, wherein R⁴ is from methyl, ethyl, propyl, butyl, pentyl, hexyl, heptyl, octyl, —OR^{4a} or —NR^{4a}R^{4b}, said methyl, ethyl, propyl, butyl, pentyl, hexyl, heptyl or octyl is optionally substituted with at least one substituents —OH, —F, —Cl, —F, —I, methyl, ethyl, propyl, butyl, pentyl, hexyl, heptyl or octyl;
R^{4a} and R^{4b} are each hydrogen, methyl, ethyl, propyl, butyl, pentyl, hexyl, heptyl or octyl; preferably R⁴ is selected from —CH₃, —OCH₃, —NHCH₃, —CHF₂, or —C(CH₃)₂OH.

30. The compound according to claim 1, wherein X¹ and X² are each independently a single bond, —CH₂—, —CH₂CH₂—, —CH₂CH₂CH₂—.

31. The compound according to claim 30, wherein when X¹ is —CH₂—, X² is —CH₂CH₂—; or when X′ is —CH₂CH₂—, X² is —CH₂—; or when X¹ is a single bond, X² is —CH₂CH₂CH₂—; or when X¹ is —CH2CH₂CH₂—, X² is a single bond.

32. The compound according to claim 1, wherein R^{X1}, R^{X2}, R^{X3}, R^{X4}, R^{X5} and R^{X6} each is selected from hydrogen, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, each of said methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl is optionally substituted with at least one substituent R^{Xa}; each of said R^{Xa} are independently oxo, —F, - Cl, -Br. —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl.

33. The compound according to claim 32, wherein R^{Xa} is selected from oxo, —F, —Cl, —Br, —I, methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl.

34. The compound according to claim 33, wherein R^{Xa} is selected from oxo, —F, —Cl, —Br, —I, methyl, ethyl, n-propyl, iso-propyl, n-butyl, sec-butyl, iso-butyl, tert-butyl, cyclopropyl, cyclobutyl, cyclopentyl.

35. The compound according to claim 1, wherein the compound is selected from

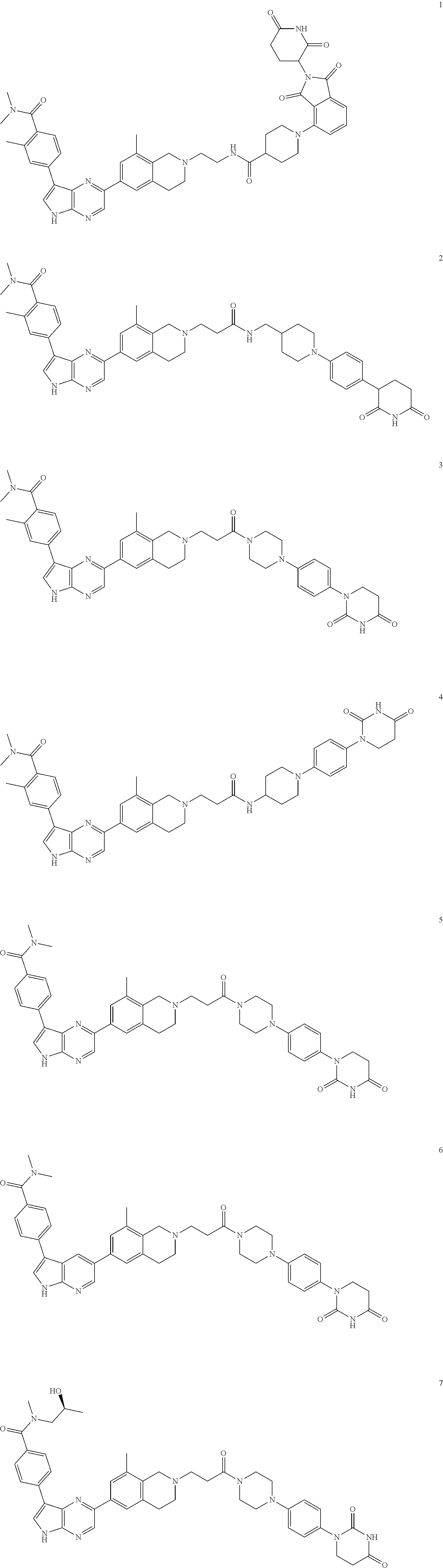

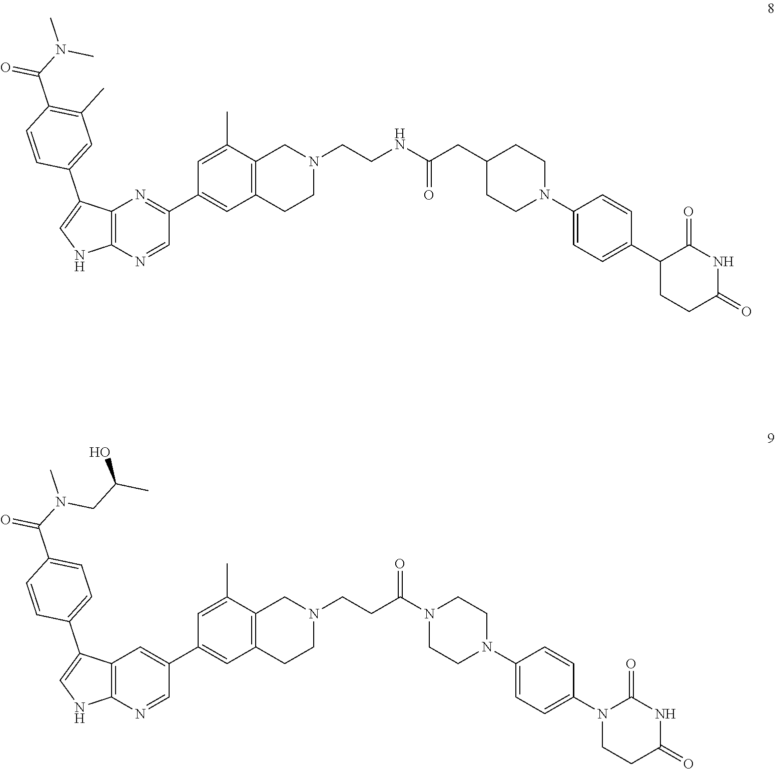 [SMILES: Cc1cc(-c2c[nH]c3ncc(-c4cc(C)c5c(c4)CCN(CCNC(=O)CC4CCN(c6ccc(C7CCC(=O)NC7=O)cc6)CC4)C5)nc23)ccc1C(=O)N(C)C ; Cc1cc(-c2cnc3[nH]cc(-c4ccc(C(=O)N(C)C[C@H](C)O)cc4)c3c2)cc2c1CN(CCC(=O)N1CCN(c3ccc(N4CCC(=O)NC4=O)cc3)CC1)CC2]

36. A pharmaceutical composition comprising the compound according to any one of claims 1-35, or a pharmaceutically acceptable salt thereof, and at least one pharmaceutically acceptable carrier or excipient.

37. A method of decreasing HPK1 activity by inhibition and/or protein degradation, which comprises administering to an individual the compound according to any one of claims 1-35, or a pharmaceutically acceptable salt thereof.

38. A method of treating a disease or disorder in a patient comprising administering to the patient a therapeutically effective amount of the compound disclosed herein, or a pharmaceutically acceptable salt thereof as an HPK1 degrader, wherein the compound disclosed herein includes the compound of claims 1-35.
